(19) United States
(12) Patent Application Publication
(10) Pub. No.: US 2025/0354079 A1
(43) Pub. Date: Nov. 20, 2025

(54) FLUORINE-CONTAINING ETHER COMPOUND, LUBRICANT FOR MAGNETIC RECORDING MEDIUM AND MAGNETIC RECORDING MEDIUM

(71) Applicant: Resonac Corporation, Tokyo (JP)

(72) Inventors: Natsumi YOSHIMURA, Tokyo (JP); Ayano ASANO, Tokyo (JP); Yutaka TANJI, Tokyo (JP); Daisuke YAGYU, Tokyo (JP); Takuya UNO, Tokyo (JP); Tsuyoshi KATO, Tokyo (JP); Naoya FUKUMOTO, Tokyo (JP)

(73) Assignee: Resonac Corporation, Tokyo (JP)

(21) Appl. No.: 18/871,179

(22) PCT Filed: Jun. 8, 2023
(86) PCT No.: PCT/JP2023/021328

§ 371 Date: Dec. 3, 2024

(30) Foreign Application Priority Data
Jun. 10, 2022 (JP) 2022-094482

(51) Int. Cl. C10M 107/38; C08G 65/331; G11B 5/72; G11B 5/725

(52) U.S. Cl. CPC C10M 107/38; C08G 65/3312; G11B 5/7257; G11B 5/7266

(57) ABSTRACT

A fluorine-containing ether compound represented by R¹—[B]-[A]-CH₂—R²[—CH₂—R³—CH₂—R²], CH₂—[C]-[D]-R⁴ ([A] is Formula (2-1), [B] is Formula (2-2), [C] is Formula (3-1), [D] is Formula (3-2), R⁴ is Formula (4), R¹ is a terminal group that may be the same as or different from R⁴, z is 1 or 2, and R² is a perfluoropolyether chain. R³ is Formula (5)).

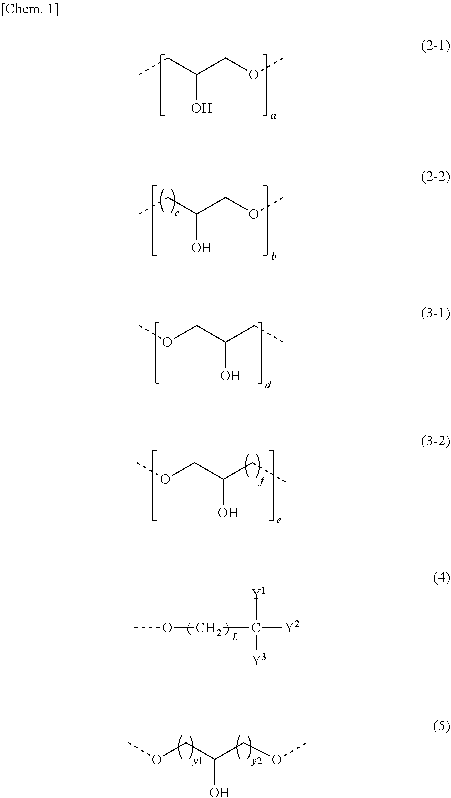

### TECHNICAL FIELD

The present invention relates to a fluorine-containing ether compound, a lubricant for magnetic recording medium and a magnetic recording medium.

Priority is claimed on Japanese Patent Application No. 2022-094482, filed Jun. 10, 2022, the content of which is incorporated herein by reference.

### BACKGROUND ART

In recent years, the amount of information processing via the Internet has increased dramatically. Accordingly, the development of recording media for storing information has been focused on. In particular, magnetic recording media, which are a type of recording media, are expected to serve as a receiver for the increased amount of information because they can store a large amount of information at a low cost.

Generally, in order to secure durability and reliability of the magnetic recording medium, a protective layer and a lubricating layer are provided on the magnetic layer (magnetic recording layer) of the magnetic recording medium. The lubricating layer arranged on the outermost surface of the magnetic recording medium is required to have various characteristics such as long-term stability, chemical substance resistance (preventing contamination with siloxane or the like), wear resistance, and heat resistance.

As a lubricant used when the lubricating layer of the magnetic recording medium is formed, for example, one containing a compound having a polar group such as a hydroxy group at the terminal of a fluorine-based polymer having a repeating structure including —CF₂— has been proposed.

For example, Patent Document 1, Patent Document 2 and Patent Document 3 disclose a fluorine-containing ether compound which contains two perfluoropolyether chains in the molecule and in which a linking group containing a secondary hydroxy group is arranged between two perfluoropolyether chains.

Patent Document 4 discloses a fluorine-containing ether compound which contains two perfluoropolyether chains in the molecule and in which a linking group containing a primary hydroxy group and a secondary hydroxy group is arranged between two perfluoropolyether chains.

Patent Document 5, Patent Document 6 and Patent Document 7 disclose a fluorine-containing ether compound which has a framework in which three perfluoropolyether chains are bonded via a linking group containing a secondary hydroxy group and a terminal group having a polar group is bonded to both sides via a methylene group (—CH₂—).

Patent Document 8 discloses a method of producing polyol (per) fluoropolyether derivatives useful as a lubricant for magnetic medium. Patent Document 8 describes that a protected triol having two protected hydroxy functional groups and one free hydroxy group is reacted with an activating agent to generate an activated protected triol, which is subjected to a nucleophilic substitution reaction with a hydroxy group arranged at the terminal of functional (per) fluoropolyether derivatives to generate protected polyol (per) fluoropolyether derivatives.

### CITATION LIST

### Patent Document

Patent Document 1: PCT International Publication No. WO2021/251335
Patent Document 2: U.S. Patent Application Publication No. 2020/0002640
Patent Document 3: PCT International Publication No. WO2016/084781
Patent Document 4: PCT International Publication No. WO2021/019998
Patent Document 5: U.S. Patent Application Publication No. 2016/0260452
Patent Document 6: PCT International Publication No. WO2018/116742
Patent Document 7: PCT International Publication No. WO2017/145995
Patent Document 8: Japanese Patent No. 5334064

### SUMMARY OF INVENTION

### Technical Problem

In recent years, due to diversity of applications of magnetic recording media, environmental resistance required for magnetic recording media has become extremely severe. Accordingly, the lubricating layer, which greatly influences the reliability and durability of the magnetic recording medium, is required to have further improved long-term stability.

As indexes for long-term stability of the lubricating layer, a pickup characteristic and a spin-off characteristic are known. Pickup is a phenomenon in which a lubricant adheres to a magnetic head as foreign matter (smear). The pickup influences flight stability of the magnetic head. Spin-off is a phenomenon in which a lubricant scatters and evaporates due to a centrifugal force and heat generated according to rotation of the magnetic recording medium. If spin-off occurs, since the film thickness of the lubricating layer is reduced, chemical resistance and wear resistance of the lubricating layer deteriorate.

In addition, in recent years, in order to increase the capacity of the magnetic recording medium, further reduction in the magnetic spacing (distance between the magnetic head and the magnetic layer of the magnetic recording medium) and increase in the rotational speed of the magnetic recording medium have been required. However, when the flying height of the magnetic head decreases, pickup is more likely to occur. In addition, when the rotational speed of the magnetic recording medium increases, spin-off is more likely to occur. In addition, when the thickness of the lubricating layer is reduced in order to reduce the raised amount of the magnetic head, the corrosion resistance of the magnetic recording medium tends to decrease.

The present invention has been made in view of the above circumstances, and an object of the present invention is to provide a fluorine-containing ether compound which can form a lubricating layer which has a strong effect of inhibiting corrosion of a magnetic recording medium and in which pickup and spin-off are less likely to occur even if the thickness is thin, and is suitably used as a material for a lubricant for magnetic recording medium.

In addition, an object of the present invention is to provide a lubricant for magnetic recording medium containing the fluorine-containing ether compound of the present invention.

In addition, an object of the present invention is to provide a magnetic recording medium which has a lubricating layer containing the fluorine-containing ether compound of the present invention, in which pickup and spin-off are less likely to occur, and which has excellent corrosion resistance.

### Solution to Problem

Specifically, the present invention relates to the following aspects.
[1] A fluorine-containing ether compound represented by the following Formula (1):

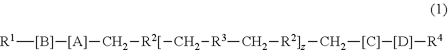

(in Formula (1), [A] is represented by the following Formula (2-1), in Formula (2-1), a is an integer of 0 to 3, [B] is represented by the following Formula (2-2), in Formula (2-2), b is an integer of 0 to 3 and c is an integer of 2 to 5, wherein a sum of the values of a and b is 1 to 3, in Formula (1), [A] and [B] may be interchanged, [C] is represented by the following Formula (3-1), in Formula (3-1), d is an integer of 0 to 2, [D] is represented by the following Formula (3-2), in Formula (3-2), e is an integer of 0 to 2, and f is an integer of 2 to 5, wherein a sum of the values of d and e is 1 or 2, in Formula (1), [C] and [D] may be interchanged, R⁴ is a branched terminal group having 3 to 30 carbon atoms and represented by the following Formula (4), in Formula (4), L represents an integer of 0 to 6, in Formula (4), Y¹ and Y² are each independently a hydrocarbon group which has only one primary hydroxy group and may contain an ether oxygen atom, in Formula (4), Y³ is a hydrocarbon group which has only one primary hydroxy group and may contain an ether oxygen atom, or a hydrogen atom, R¹ is a terminal group that may be the same as or different from R⁴, a branched terminal group having 3 to 30 carbon atoms represented by Formula (4), an organic group having 1 to 30 carbon atoms, which has an ether oxygen atom at the terminal bonded to [A] or [B], or a hydroxy group, z represents 1 or 2, R² is a perfluoropolyether chain, some or all of two or three R²'s may be the same as or different from each other, R³ is a divalent linking group represented by the following Formula (5), in Formula (5), y1 is an integer of 1 to 3, and y2 is an integer of 1 to 3, in Formula (5), a dotted line bonded to the oxygen atom on the left side indicates a bond with a methylene group on the side of R¹, and a dotted line bonded to the oxygen atom on the right side indicates a bond with a methylene group on the side of R⁴, and when z is 2, two R³'s may be the same as or different from each other).

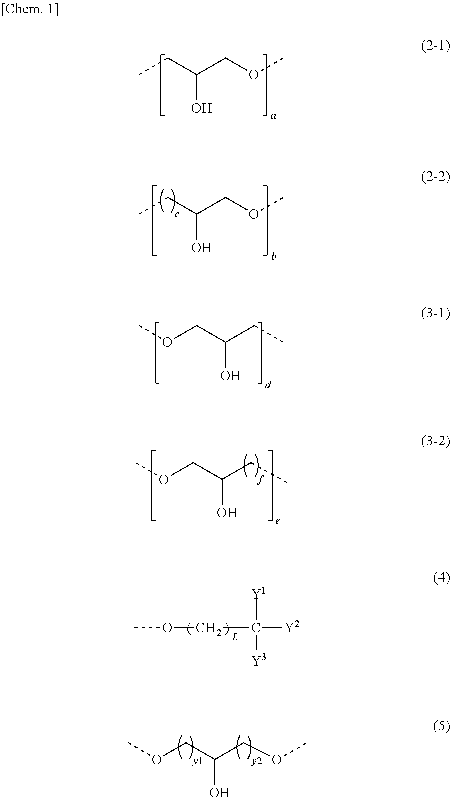

[2] The fluorine-containing ether compound according to [1],
   wherein Formula (4) representing R⁴ in Formula (1) is any of the following Formulae (6-1) to (6-3):

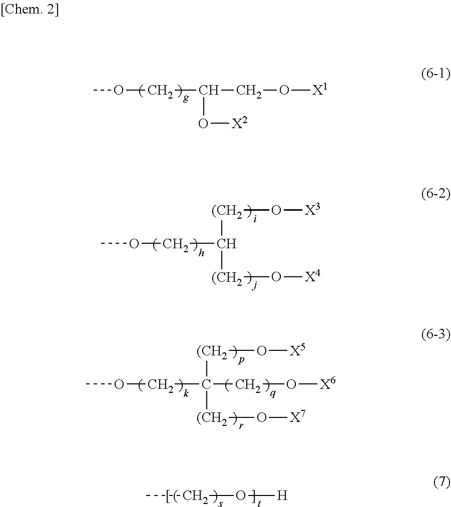

(in Formula (6-1), g represents an integer of 1 to 6, X1 and X2 are represented by Formula (7), and X¹ and X² may be the same as or different from each other)
(in Formula (6-2), h represents an integer of 0 to 6, i and j each independently represent an integer of 1 to 6, X³ and X⁴ represent a hydrogen atom or are represented by Formula (7), and X³ and X⁴ may be the same as or different from each other)
(in Formula (6-3), k represents an integer of 0 to 6, p, q and r each independently represent an integer of 1 to 6, X⁵, X⁶ and X⁷ represent a hydrogen atom or are represented by Formula (7), and X⁵, X⁶ and X⁷ may be different from each other, or some or all of them may be the same)
(in Formula (7), s represents an integer of 2 to 6, and t represents 1 or 2).
[3] The fluorine-containing ether compound according to [1] or [2],
   wherein, in Formula (1), R¹ is a branched terminal group having 3 to 30 carbon atoms represented by Formula (4).
[4] The fluorine-containing ether compound according to [2],
   wherein, in Formula (1), both R¹ and R⁴ are a branched terminal group of any of

### Formulae (6-1) to (6-3).

[5] The fluorine-containing ether compound according to any one of [1] to [4],
   wherein, in Formula (1), R¹—[B]-[A]-and-[C]-[D]-R⁴ are the same.
[6] The fluorine-containing ether compound according to any one of [1] to [5],
   wherein, in Formula (1), z is 2, and atoms contained in two R³'s are arranged symmetrically with respect to R² arranged in the center of a chain structure of a molecule.
[7] The fluorine-containing ether compound according to any one of [1] to [6],
   wherein, in Formula (1), R¹ is represented by the following Formula (8).

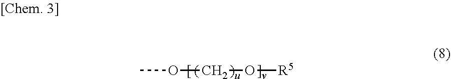

(in Formula (8), u represents an integer of 2 to 6, v represents 0 or 1, and R⁵ is any of a hydrogen atom, an optionally substituted alkyl group containing no hydroxy group, and an organic group having at least one double bond or triple bond, provided that the alkyl group and the organic group may be linear or branched).
[8] The fluorine-containing ether compound according to [7],
   wherein, in Formula (8), R⁵ is an alkyl group having 1 to 6 carbon atoms.
[9] The fluorine-containing ether compound according to [7],
   wherein, in Formula (8), R⁵ is an alkyl group having a substituent and having 1 to 6 carbon atoms, and the substituent is a fluoro group or a cyano group.
[10] The fluorine-containing ether compound according to [7],
   wherein, in Formula (8), R⁵ is any of an aromatic hydrocarbon-containing organic group having 6 to 12 carbon atoms, an aromatic heterocycle-containing organic group having 3 to 10 carbon atoms, an alkenyl group having 2 to 8 carbon atoms, and an alkynyl group having 3 to 8 carbon atoms.
[11] The fluorine-containing ether compound according to [7],
   wherein, in Formula (8), R⁵ is one group selected from the group consisting of a methyl group, ethyl group, n-propyl group, isopropyl group, 2,2,2-trifluoroethyl group, 2,2,3,3,3-pentafluoropropyl group, 2,2,2,2,2,2-hexafluoroisopropyl group, 2-cyanoethyl group, 3-cyanopropyl group, 4-cyanobutyl group, phenyl group, methoxyphenyl group, cyanophenyl group, phenethyl group, thienylethyl group, N-methylpyrazolylmethyl group, allyl group, 3-butenyl group, 4-pentenyl group, propargyl group, 3-butynyl group, and 4-pentynyl group.
[12] The fluorine-containing ether compound according to [7],
   wherein, in Formula (8), R⁵ is a hydrogen atom.
[13] The fluorine-containing ether compound according to any one of [1] to [12],
   wherein, in Formula (1), two or three R²'s are each independently a perfluoropolyether chain represented by the following Formula (9):

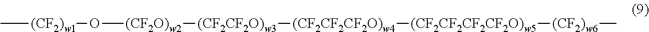

(in Formula (9), w2, w3, w4, and w5 indicate an average degree of polymerization and each independently represent 0 to 20; provided that all of w2, w3, w4, and w5 are not 0 at the same time, w1 and w6 are an average value representing the number of CF₂'s and each independently represent 1 to 3, and the arrangement order of repeating units (CF₂O), (CF₂CF₂O), (CF₂CF₂CF₂O), and (CF₂CF₂CF₂CF₂O) in Formula (9) is not particularly limited).
[14] The fluorine-containing ether compound according to any one of [1] to [12],
   wherein, in Formula (1), two or three R²'s are each independently any one selected from among perfluoropolyether chains represented by the following Formulae (10-1) to (10-4):

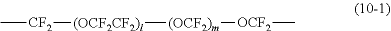

(in Formula (10-1), l and m indicate an average degree of polymerization, 1 represents 0.1 to 20, and m represents 0 to 20)

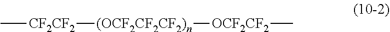

(in Formula (10-2), n indicates an average degree of polymerization and represents 0.1 to 15)

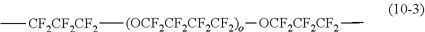

(in Formula (10-3), o indicates an average degree of polymerization and represents 0.1 to 10)

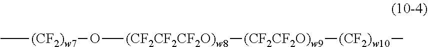

(in Formula (10-4), w8 and w9 indicate an average degree of polymerization and each independently represent 0.1 to 20. w7 and w10 are an average value representing the number of CF₂'s and each independently represent 1 to 2).
[15] The fluorine-containing ether compound according to any one of [1] to [14],
   wherein the number-average molecular weight is in a range of 500 to 10,000.
[16] A lubricant for magnetic recording medium including the fluorine-containing ether compound according to any one of [1] to [15].
[17] A magnetic recording medium in which at least a magnetic layer, a protective layer, and a lubricating layer are sequentially provided on a substrate,
   wherein the lubricating layer contains the fluorine-containing ether compound according to any one of [1] to [15].
[18] The magnetic recording medium according to [17],
   wherein the average film thickness of the lubricating layer is 0.5 nm to 2.0 nm.

### Advantageous Effects of Invention

The fluorine-containing ether compound of the present invention is the compound represented by Formula (1), and is suitable as a material for a lubricant for magnetic recording medium.

The lubricant for magnetic recording medium of the present invention contains the fluorine-containing ether compound of the present invention. Therefore, it is possible to form a lubricating layer which has a strong effect of inhibiting corrosion of a magnetic recording medium, has favorable adhesion to the protective layer, and can minimize the occurrence of pickup and spin-off even if the thickness is thin.

The magnetic recording medium of the present invention has a lubricating layer which has favorable adhesion to the protective layer, can minimize the occurrence of pickup and spin-off, and provides excellent corrosion resistance. Therefore, it has excellent reliability and durability.

### BRIEF DESCRIPTION OF DRAWINGS

FIG. 1 is a schematic cross-sectional view showing an example of a magnetic recording medium according to one embodiment of the present invention.

### DESCRIPTION OF EMBODIMENTS

In order to achieve the above objects, the inventors focused on the relationship between the molecular structure of the fluorine-containing ether compound contained in the lubricating layer and the protective layer, and conducted extensive studies as shown below.

In the related art, as a material for a lubricant for magnetic recording medium (hereinafter sometimes abbreviated as a “lubricant”) applied to the surface of a protective layer, a fluorine-containing ether compound having a polar group such as a hydroxy group at a terminal of a chain structure is used. However, a lubricating layer formed using a conventional lubricant may not have sufficient adhesion to the protective layer and/or an effect of inhibiting corrosion of a magnetic recording medium. In addition, the inventors conducted extensive studies and as a result, found that, when adhesion of the lubricating layer to the protective layer is insufficient, pickup and spin-off are likely to occur.

Thus, the inventors additionally conducted extensive studies in order to improve the adhesion of the lubricating layer to the protective layer and the effect of inhibiting corrosion of a magnetic recording medium. As a result, it was found that it was necessary to use a fluorine-containing ether compound having a hydroxy group and providing the following functions <1> to <3> as a lubricant.
<1> The hydroxy group in the perfluoropolyether (hereinafter sometimes referred to as PFPE)-based compound effectively participates in bonding with the active sites on the protective layer.
<2> The hydroxy group in the PFPE-based compound participates in the formation of intermolecular hydrogen bonds within the PFPE-based compound.
<3> Sufficient hydrophobicity is obtained by inclusion of perfluoropolyether chains (PFPE chains), and the PFPE chains are not too far away from the protective layer.

Therefore, the inventors conducted extensive studies regarding the molecular structure of the fluorine-containing ether compound that can effectively obtain the functions <1> to <3>.

As a result, it was found that it is sufficient to use a fluorine-containing ether compound in which two or three perfluoropolyether chains (PFPE chains) are bonded via a methylene group (—CH₂—) and a specific divalent linking group having a secondary hydroxy group, a specific linking group having a secondary hydroxy group is bonded to perfluoropolyether chains at both ends via a methylene group (—CH₂—), and a branched terminal group having a plurality of primary hydroxy groups is arranged at at least one terminal. Then, it was confirmed that a lubricating layer containing such a fluorine-containing ether compound has a strong effect of inhibiting corrosion of a magnetic recording medium and can minimize the occurrence of pickup and spin-off even if the thickness is thin, and the present invention was completed.

Hereinafter, a fluorine-containing ether compound, a lubricant for magnetic recording medium and a magnetic recording medium of the present invention will be described in detail. Here, the present invention is not limited only to the following embodiments. For example, the present invention is not limited only to the following examples, and numbers, amounts, ratios, compositions, types, positions, materials, configurations and the like can be added, omitted, substituted, and changed without departing from the spirit and scope of the present invention.

### [Fluorine-Containing Ether Compound]

A fluorine-containing ether compound of the present embodiment is represented by the following Formula (1).

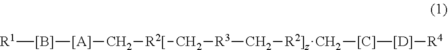

(in Formula (1), [A] is represented by the following Formula (2-1), in Formula (2-1), a is an integer of 0 to 3, [B] is represented by the following Formula (2-2), in Formula (2-2), b is an integer of 0 to 3 and c is an integer of 2 to 5, wherein a sum of the values of a and b is 1 to 3, in Formula (1), [A] and [B] may be interchanged, [C] is represented by the following Formula (3-1), in Formula (3-1), d is an integer of 0 to 2, [D] is represented by the following Formula (3-2), in Formula (3-2), e is an integer of 0 to 2, and f is an integer of 2 to 5, wherein a sum of the values of d and e is 1 or 2, in Formula (1), [C] and [D] may be interchanged, R⁴ is a branched terminal group having 3 to 30 carbon atoms and represented by the following Formula (4), in Formula (4), L represents an integer of 0 to 6, in Formula (4), Y¹ and Y² are each independently a hydrocarbon group which has only one primary hydroxy group and may contain an ether oxygen atom, in Formula (4), Y³ is a hydrocarbon group which has only one primary hydroxy group and may contain an ether oxygen atom, or a hydrogen atom, R¹ is a terminal group that may be the same as or different from R⁴, a branched terminal group having 3 to 30 carbon atoms represented by Formula (4), an organic group having 1 to 30 carbon atoms, which has an ether oxygen atom at the terminal bonded to [A] or [B], or a hydroxy group, z represents 1 or 2, R² is a perfluoropolyether chain, some or all of two or three R²'s may be the same as or different from each other, R³ is a divalent linking group represented by the following Formula (5), in Formula (5), y1 is an integer of 1 to 3, and y2 is an integer of 1 to 3, in Formula (5), a dotted line bonded to the oxygen atom on the left side indicates a bond with a methylene group on the side of R¹, and a dotted line bonded to the oxygen atom on the right side indicates a bond with a methylene group on the side of R⁴, and when z is 2, two R³'s may be the same as or different from each other).

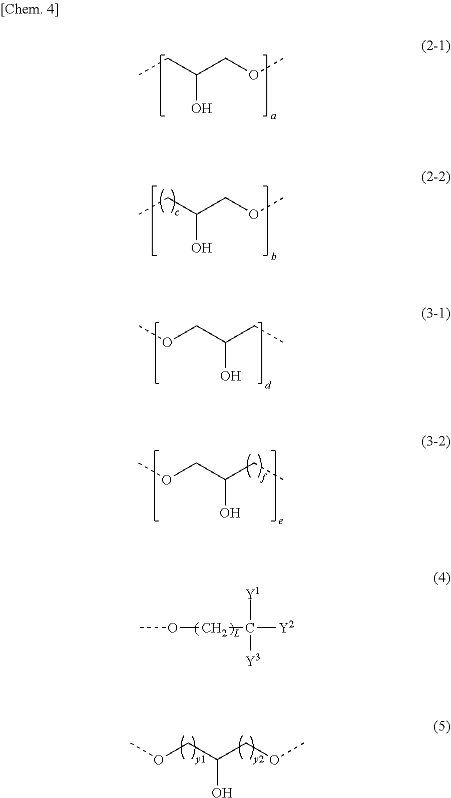

### (Linking Group Represented by —[B]-[A]-)

In the fluorine-containing ether compound represented by Formula (1) according to the present embodiment, [A] is represented by Formula (2-1), and [B] is represented by Formula (2-2). In Formula (1), [A] and [B] are a divalent linking group. In Formula (1), [A] and [B] may be interchanged. a in Formula (2-1) and b in Formula (2-2) are an integer of 0 to 3. However, a sum of the values of a and b is 1 to 3.

In consideration of availability of raw materials and ease of synthesis, Formula (2-1) and Formula (2-2) are preferably a combination in which a is 1 and b is 0 or a combination in which a is 0 and b is 1.

In addition, in consideration of the adhesion to the protective layer, Formula (2-1) and Formula (2-2) are preferably a combination in which a is 2 and b is 0 or a combination in which a is 1 and b is 1. Particularly, when a is 2 and b is 0, in the fluorine-containing ether compound, the direction in which two hydroxy groups in Formula (2-1) are arranged is sterically the same direction as the extending direction of PFPE chains, and the two hydroxy groups in Formula (2-1) tend to be easily adsorbed to the protective layer. In addition, when a and b are 1, and the bonding order of [A] and [B] is-[A]-[B]— from the side of R¹, the distance between hydroxy groups contained in the -[A]-[B]— structure becomes longer. Therefore, it is possible to reduce the number of intramolecular hydrogen bonds of the fluorine-containing ether compound represented by Formula (1) and increase the affinity with the protective layer.

In Formula (2-2), c is an integer of 2 to 5. When b is an integer of 1 to 3, c is preferably an integer of 2 to 4 and most preferably 2. This is because the —[B]-[A]— structure does not contain too many carbon atoms, and a fluorine-containing ether compound which can form a lubricating layer which has better adhesion to the protective layer and can minimize the occurrence of pickup and spin-off is obtained.

### (Linking Group Represented by —[C]-[D]-)

In the fluorine-containing ether compound represented by Formula (1) according to the present embodiment, [C] is represented by Formula (3-1), and [D] is represented by Formula (3-2). In Formula (1), [C] and [D] are a divalent linking group. In Formula (1), [C] and [D] may be interchanged. d in Formula (3-1) and e in Formula (3-2) are an integer of 0 to 2. However, a sum of the values of d and e is 1 or 2.

In consideration of availability of raw materials and ease of synthesis, Formula (3-1) and Formula (3-2) are preferably a combination in which d is 1 and e is 0 or a combination in which d is 0 and e is 1.

In addition, in consideration of the adhesion to the protective layer, Formula (3-1) and Formula (3-2) are preferably a combination in which d is 2 and e is 0 or a combination in which d is 1 and e is 1. Particularly, when d is 2 and e is 0, in the fluorine-containing ether compound, the direction in which two hydroxy groups in Formula (3-1) are arranged is sterically the same direction as the extending direction of PFPE chains, and the two hydroxy groups in Formula (3-1) tend to be easily adsorbed to the protective layer. In addition, when d and e are 1, and the bonding order of [C] and [D] is-[D]-[C]— from the side of R², the distance between hydroxy groups contained in the -[D]-[C]— structure becomes longer. Therefore, it is possible to reduce the number of intramolecular hydrogen bonds of the fluorine-containing ether compound represented by Formula (1) and increase the affinity with the protective layer.

In Formula (3-2), f is an integer of 2 to 5. When e is an integer of 1 to 2, f is preferably an integer of 2 to 3 and most preferably 2. This is because the —[C]-[D]-structure does not contain many carbon atoms, and a fluorine-containing ether compound which can form a lubricating layer which has better adhesion to the protective layer and can minimize the occurrence of pickup and spin-off is obtained.

### (Branched Terminal Group Represented by R⁴)

In Formula (1), R⁴ is a branched terminal group having 3 to 30 carbon atoms and represented by Formula (4). The number of carbon atoms in R⁴ is preferably 3 to 20 and more preferably 3 to 12. The number of carbon atoms in R⁴ may be 3 to 5, 5 to 10, to 15 or the like. When the number of carbon atoms in R⁴ is 3 to 12, it is possible to minimize an increase in the surface free energy of all molecules due to a decrease in the proportion of fluorine atoms in the fluorine-containing ether compound molecules.

Formula (4) is a branched terminal group containing two or three primary hydroxy groups and having a carbon atom as a branch point. A plurality of primary hydroxy groups contained in R⁴ participate in the formation of intermolecular hydrogen bonds within the fluorine-containing ether compound.

In Formula (4), L represents an integer of 0 to 6. In Formula (4), Y¹ and Y² are each independently a hydrocarbon group containing only one primary hydroxy group and optionally containing an ether oxygen atom. The hydrocarbon groups represented by Y¹ and Y² may be linear or branched, and preferably contain no secondary hydroxy group or tertiary hydroxy group. Y³ is a hydrocarbon group containing only one primary hydroxy group and optionally containing an ether oxygen atom or a hydrogen atom. The hydrocarbon group represented by Y³ may be linear or branched, and preferably contains no secondary hydroxy group or tertiary hydroxy group.

R⁴ preferably contains three or more ether bonds (—O—). In this case, since R⁴ has appropriate flexibility, the lubricating layer containing the fluorine-containing ether compound represented by Formula (1) has better adhesion to the protective layer.

When R⁴ has a plurality of ether bonds, adjacent ether bonds are preferably bonded to each other via a linking group in which two or more carbon atoms are linked. In this case, the distance between adjacent ether bonds is appropriate, and the fluorine-containing ether compound is unlikely to aggregate.

Formula (4) representing R⁴ is preferably a branched terminal group of any of the following Formulae (6-1) to (6-3). When R⁴ is a branched terminal group of any of Formulae (6-1) to (6-3), carbon atoms to which the primary hydroxy groups contained in R⁴ are bonded are bonded via a linking group containing a methine group and/or methylene group and an ether bond. Therefore, the distance between adjacent primary hydroxy groups contained in R⁴ is appropriate, and a plurality of primary hydroxy groups contained in R⁴ are arranged to facilitate formation of intermolecular hydrogen bonds within the fluorine-containing ether compound. Furthermore, when R⁴ is a branched terminal group of any of the following Formulae (6-1) to (6-3), the molecular weight of R⁴ is large, and thus it is possible to minimize an increase in the surface free energy of all molecules due to a decrease in the proportion of fluorine atoms in the fluorine-containing ether compound molecules. R⁴ is more preferably Formula (6-1) or (6-2) because it is possible to minimize an increase in the surface free energy of all molecules.

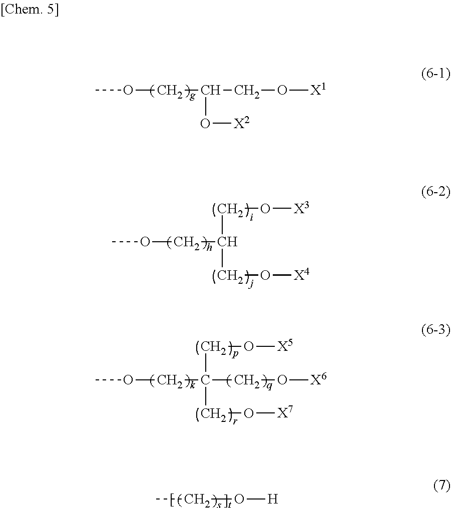

(in Formula (6-1), g represents an integer of 1 to 6, X¹ and X² are represented by Formula (7), and X¹ and X² may be the same as or different from each other)
(in Formula (6-2), h represents an integer of 0 to 6, i and j each independently represent an integer of 1 to 6, X³ and X⁴ represent a hydrogen atom or are represented by Formula (7), and X³ and X⁴ may be the same as or different from each other)
(in Formula (6-3), k represents an integer of 0 to 6, p, q and r each independently represent an integer of 1 to 6, X⁵, X⁶ and X⁷ represent a hydrogen atom or are represented by Formula (7), and X⁵, X⁶ and X⁷ may be different from each other, or some or all of them may be the same)
(in Formula (7), s represents an integer of 2 to 6, and t represents 1 or 2).

In Formula (6-1), g represents an integer of 1 to 6. g is preferably an integer of 1 to 4 and more preferably 1 or 2 because it is easy to secure the proportion of fluorine atoms in the fluorine-containing ether compound molecules. In addition, when g is an integer of 1 to 4, the terminal movement does not become too large due to a large number of carbon atoms in R⁴. Therefore, a fluorine-containing ether compound can form a lubricating layer which has better adhesion to the protective layer and can minimize the occurrence of pickup and spin-off, which is preferable.

X¹ and X² are represented by Formula (7). X¹ and X² may be the same as or different from each other.

In Formula (6-2), h represents an integer of 0 to 6. h is preferably an integer of 0 to 4 and more preferably an integer of 0 to 2 because it is easy to secure the proportion of fluorine atoms in the fluorine-containing ether compound molecules. In addition, when h is an integer of 0 to 5, the terminal movement does not become too large due to a large number of carbon atoms in R⁴. Therefore, a fluorine-containing ether compound can form a lubricating layer which has better adhesion to the protective layer and can minimize the occurrence of pickup and spin-off, which is preferable.

In Formula (6-2), i and j each independently represent an integer of 1 to 6. i and j are each independently preferably an integer of 1 to 4 and more preferably 1 or 2 because it is easy to secure the proportion of fluorine atoms in the fluorine-containing ether compound molecules. i and j may be the same as or different from each other. i and j are preferably the same since it is easy to produce the fluorine-containing ether compound.

X³ and X⁴ represent a hydrogen atom or are represented by Formula (7). X³ and X⁴ may be the same as or different from each other.

In Formula (6-3), k represents an integer of 0 to 6. k is preferably an integer of 0 to 4 and more preferably an integer of 0 to 2 because it is easy to secure the proportion of fluorine atoms in the fluorine-containing ether compound molecules.

p, q and r each independently represent an integer of 1 to 6. p, q and r are each independently preferably an integer of 1 to 4 and more preferably 1 or 2 because it is easy to secure the proportion of fluorine atoms in the fluorine-containing ether compound molecules. p, q and r may be different from each other, or some or all of them may be the same. p, q and r are preferably all the same since it is easy to produce the fluorine-containing ether compound.

X⁵, X⁶ and X⁷ represent a hydrogen atom or are represented by Formula (7). X⁵, X⁶ and X⁷ may be different from each other, or some or all of them may be the same.

In Formula (7), s represents an integer of 2 to 6. s is preferably an integer of 2 to 4 and preferably 2 or 3 because it is easy to secure the proportion of fluorine atoms in the fluorine-containing ether compound molecules.

t represents 1 or 2. When t is 2, s's in [—(CH₂) s-O-] may be the same as or different from each other. t is preferably 1 because it is easy to secure the proportion of fluorine atoms in the fluorine-containing ether compound molecules.

In Formula (7) as X² in Formula (6-1), preferably, s is 3 or more, and/or t is 2, and in both Formula (7) as X¹ in Formula (6-1) and Formula (7) as X² in Formula (6-1), more preferably, s is 3 or more and/or t is 2. This is because the distance from the carbon atom that is a branch point in Formula (6-1) to the primary hydroxy group is not too short and is appropriate, and a fluorine-containing ether compound which can form a lubricating layer that can more effectively minimize the occurrence of pickup and spin-off is obtained.

Specifically, —[C]-[D]-R⁴ in Formula (1) is preferably a structure represented by any of the following Formulae (11-1) to (11-25), and more preferably a structure represented by any of the following Formulae (11-1) to (11-5), (11-7) to (11-10), (11-13) to (11-21), (11-24), and (11-25) because a fluorine-containing ether compound which can form a lubricating layer that can effectively minimize the occurrence of pickup and spin-off is obtained.

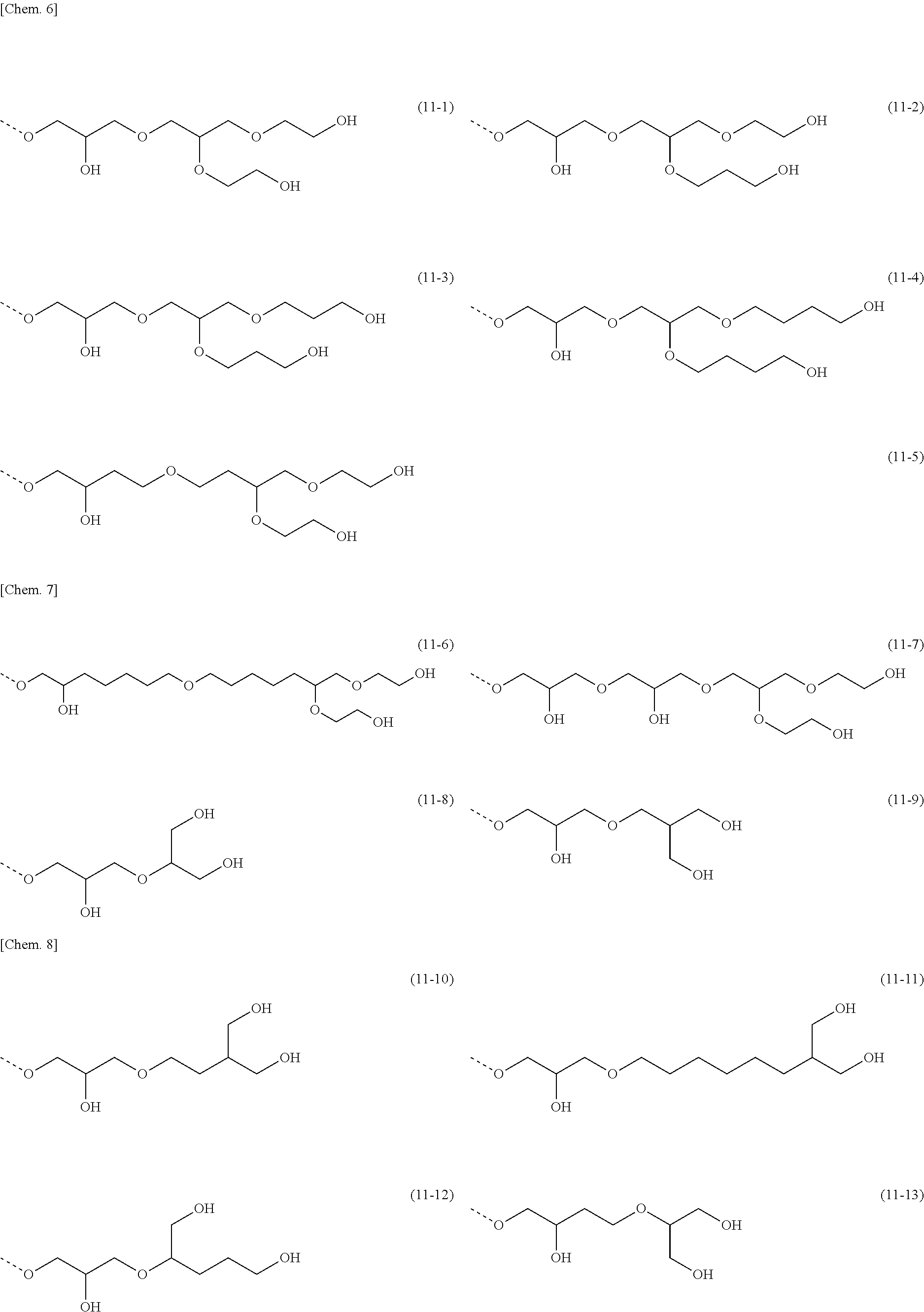

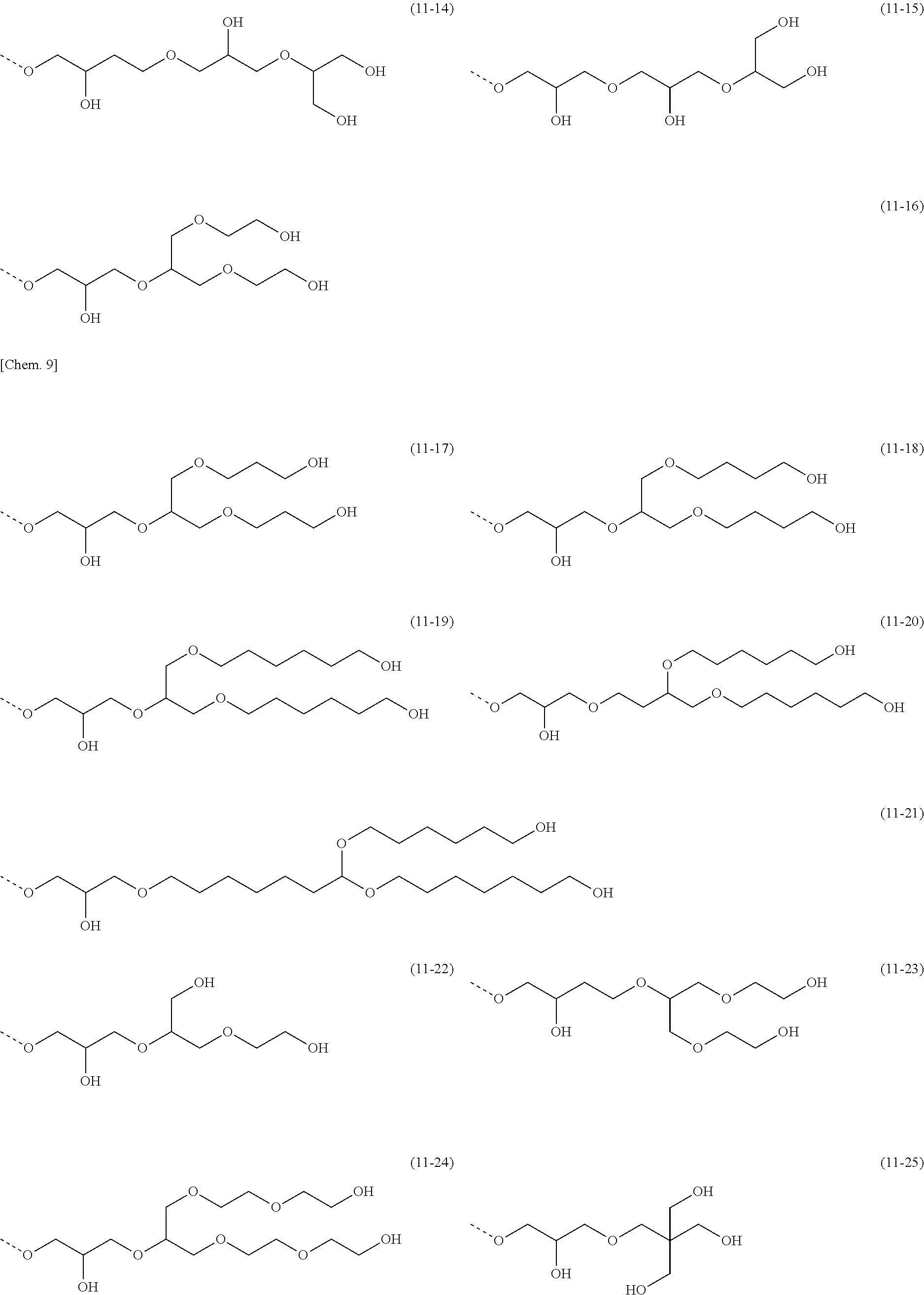

### (Terminal Group Represented by R¹)

The terminal group represented by R¹ in (1) is a terminal group which may be the same as or different from R⁴, and is a branched terminal group having 3 to 30 carbon atoms represented by Formula (4), an organic group having 1 to 30 carbon atoms and having an ether oxygen atom at the terminal bonded to [A] or [B], or a hydroxy group. The terminal group represented by R¹ can be appropriately selected depending on the performance required for the lubricant containing the fluorine-containing ether compound and the like.

When R¹ is a terminal group having 3 to 30 carbon atoms represented by Formula (4), R¹ is a branched terminal group containing two or three primary hydroxy groups and having a carbon atom as a branch point. Therefore, a plurality of primary hydroxy groups contained in R¹ participate in the formation of intermolecular hydrogen bonds within the fluorine-containing ether compound, and thus the intermolecular hydrogen bonds within the fluorine-containing ether compound are further strengthened.

When R¹ is a terminal group represented by Formula (4), Formula (4) representing R¹ is preferably a branched terminal group of any of Formulae (6-1) to (6-3). In this case, preferable values of g in Formula (6-1), h to j in Formula (6-2), k, and p to r in Formula (6-3), and s and t in Formula (7) are the same as those when R⁴ is a branched terminal group of any of Formulae (6-1) to (6-3).

When R¹ is a terminal group represented by Formula (4), -[A]-[B]—R¹ is specifically preferably a structure represented by Formulae (11-1) to (11-25).

In Formula (1), when R¹ is a terminal group represented by Formula (4), both R¹ and R⁴ are more preferably a branched terminal group of any of Formulae (6-1) to (6-3).

In Formula (1), when R¹ is a terminal group represented by Formula (4), R¹ and R⁴ are preferably the same, and both R¹ and R⁴ are more preferably a branched terminal group of any of Formulae (6-1) to (6-3).

In the fluorine-containing ether compound represented by Formula (1) according to the present embodiment, when R¹ is not a branched terminal group represented by Formula (4), R¹ is an organic group having 1 to 30 carbon atoms and having an ether oxygen atom at the terminal bonded to [A] or [B] or a hydroxy group. Specifically, R¹ is preferably a terminal group represented by the following Formula (8). In this case, the molecular weight of R¹ is large, and thus it is possible to minimize an increase in the surface free energy of all molecules due to a decrease in the proportion of fluorine atoms in the fluorine-containing ether compound molecules.

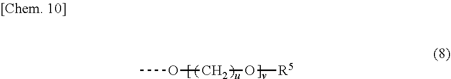

(in Formula (8), u represents an integer of 2 to 6, v represents 0 or 1, and R⁵ is any of a hydrogen atom, an optionally substituted alkyl group containing no hydroxy group, and an organic group having at least one double bond or triple bond, provided that the alkyl group and the organic group may be linear or branched).

In Formula (8), u represents an integer of 2 to 6, v represents 0 or 1. When v in

Formula (8) is 0, it is possible to more effectively minimize an increase in the surface free energy of all molecules due to a decrease in the proportion of fluorine atoms in the fluorine-containing ether compound molecules. When v is 1, the ether bond contained in Formula (8) imparts flexibility to the fluorine-containing ether compound represented by Formula (1), and thus adsorption to the protective layer becomes easier.

In addition, when v in Formula (8) is 1, since u is an integer of 2 to 6, the terminal group represented by R¹ is chemically stable and is less likely to be decomposed. u is preferably an integer of 2 to 4 and more preferably 2 or 3. When u is 2 or 3, it is possible to minimize an increase in the surface free energy of all molecules due to a decrease in the proportion of fluorine atoms in the fluorine-containing ether compound molecules.

In Formula (8), R⁵ is any of a hydrogen atom, an optionally substituted alkyl group containing no hydroxy group, and an organic group having at least one double bond or triple bond.

When R⁵ is a hydrogen atom, R⁵ forms a hydroxy group together with an oxygen atom in Formula (8). When v in Formula (8) is 1, R¹ represented by Formula (8) is an alkoxy group having a hydroxy group at the terminal. When v in Formula (8) is 0, R¹ represented by Formula (8) is a hydroxy group.

When R⁵ is a hydrogen atom and v in Formula (8) is 1, preferable specific examples of R¹ represented by Formula (8) include-O—CH₂CH₂—OH (in Formula (8), u is 2), and —O—CH₂CH₂CH₂—OH (in Formula (8), u is 3).

When R⁵ is a hydrogen atom and v in Formula (8) is 0 (that is, when R¹ is a hydroxy group), R¹ may be bonded to [A] represented by Formula (2-1) or may be bonded to [B] represented by Formula (2-2). When R¹ is bonded to [B], this is preferable because the distance between the hydroxy group represented by R¹ and the adjacent hydroxy group becomes more appropriate.

When R⁵ is an optionally substituted alkyl group containing no hydroxy group, examples of substituents include a fluoro group, a cyano group, and an alkoxy group. The substituent does not include a substituent containing a hydroxy group such as a hydroxyalkoxy group.

The optionally substituted alkyl group is preferably an alkyl group having no substituent and having 1 to 6 carbon atoms or an alkyl group having a substituent and having 1 to 6 carbon atoms. The substituent of the alkyl group having a substituent and having 1 to 6 carbon atoms is preferably a fluoro group or a cyano group. The alkyl group having a substituent and having 1 to 6 carbon atoms may be one in which one more hydrogen atoms of the alkyl group are substituted with a substituent or one in which all hydrogen atoms of the alkyl group are substituted with a substituent.

The alkyl groups included in the alkyl group having no substituent and having 1 to 6 carbon atoms and the alkyl group having a substituent and having 1 to 6 carbon atoms may be linear or branched. Specific examples of alkyl groups include a methyl group, ethyl group, n-propyl group, isopropyl group, n-butyl group, sec-butyl group, isobutyl group, tert-butyl group, n-pentyl group and its structural isomers, n-hexyl group and its structural isomers.

Examples of alkyl groups having 1 to 6 carbon atoms in which one more hydrogen atoms are substituted with a fluoro group include a trifluoromethyl group, perfluoroethyl group, perfluoropropyl group, perfluorobutyl group, perfluoropentyl group, perfluorohexyl group, 2,2,2-trifluoroethyl group, 2,2,3,3,3-pentafluoropropyl group, 2,2,2,2,2,2-hexafluoroisopropyl group, 2,2,3,3,4,4,4-heptafluorobutyl group, 2,2,3,3,4,4,5,5,5-nonafluoropentylgroup, and 2,2,3,3,4,4,5,5,6,6,6-undecafluorohexyl group.

The number of cyano groups in the alkyl group having 1 to 6 carbon atoms in which one more hydrogen atoms are substituted with a cyano group may be 1 or 2 or more. When the number of cyano groups is large, since the polarity of the fluorine-containing ether compound becomes too high, the number of cyano groups is preferably 2 or less, and most preferably 1.

Examples of alkyl groups having 1 to 6 carbon atoms in which one more hydrogen atoms are substituted with a cyano group include a 2-cyanoethyl group, 3-cyanopropyl group, 4-cyanobutyl group, 5-cyanopentyl group, 6-cyanohexyl group, 2-cyano-1-methylethyl group, and 2,2′-dicyanoisopropyl group.

An organic group having at least one double bond or triple bond is preferably any of an aromatic hydrocarbon-containing organic group having 6 to 12 carbon atoms, an aromatic heterocycle-containing organic group having 3 to 10 carbon atoms, an alkenyl group having 2 to 8 carbon atoms, and an alkynyl group having 3 to 8 carbon atoms. The organic group having at least one double bond or triple bond may be linear or branched. The organic group having at least one double bond or triple bond may have a substituent containing no hydroxy group.

Examples of aromatic hydrocarbon-containing organic groups having 6 to 12 carbon atoms include a phenyl group, methoxyphenyl group, dimethoxyphenyl group, cyanophenyl group, dicyanophenyl group, fluorinated phenyl group, naphthyl group, methoxynaphthyl group, benzyl group, methoxybenzyl group, phenethyl group, methoxyphenethyl group, fluorinated phenethyl group, naphthylmethyl group, and naphthylethyl group. When the aromatic hydrocarbon has a substituent, the position at which the substituent is bonded may be arbitrary.

Examples of aromatic heterocycle-containing organic groups having 3 to 10 carbon atoms include a pyrrolyl group, pyrazolyl group, methylpyrazolylmethyl group, imidazolyl group, furyl group, furfuryl group, oxazolyl group, isooxazolyl group, thienyl group, thienylmethyl group, thienylethyl group, thiazolyl group, methylthiazolylethylgroup, isothiazolyl group, pyridyl group, pyrimidinyl group, pyridazinyl group, pyrazinyl group, indolinyl group, benzofuranyl group, benzothienyl group, benzoimidazolyl group, benzooxazolyl group, benzothiazolyl group, benzopyrazolyl group, benzoisooxazolyl group, benzoisothiazolyl group, quinolyl group, isoquinolyl group, quinazolinyl group, quinoxalinyl group, phthalazinyl group, and cinnolinyl group.

Examples of alkenyl groups having 2 to 8 carbon atoms include a vinyl group, allyl group, 1-propenyl group, isopropenyl group, 3-butenyl group and its structural isomers, 4-pentenyl group and its structural isomers, 5-hexenyl group and its structural isomers, 6-heptenyl group and its structural isomers, 7-octenyl group and its structural isomers.

Examples of alkynyl groups having 3 to 8 carbon atoms include a 1-propynyl group, propargyl group, 3-butynyl group and its structural isomers, 4-pentynyl group and its structural isomers, 5-hexynyl group and its structural isomers, 6-heptynyl group and its structural isomers, 7-octynyl group and its structural isomers.

In consideration of availability and/or ease of synthesis, R⁵ in Formula (8) is preferably one group selected from the group consisting of a hydrogen atom, methyl group, ethyl group, n-propyl group, isopropyl group, 2,2,2-trifluoroethyl group, 2,2,3,3,3-pentafluoropropyl group, 2,2,2,2,2,2-hexafluoroisopropyl group, 2-cyanoethyl group, 3-cyanopropyl group, 4-cyanobutyl group, phenyl group, methoxyphenyl group, cyanophenyl group, phenethyl group, thienylethyl group, N-methylpyrazolylmethyl group, allyl group, 3-butenyl group, 4-pentenyl group, propargyl group, 3-butynyl group, and 4-pentynyl group. Among these, one group selected from the group consisting of a hydrogen atom, methyl group, ethyl group, n-propyl group, 2,2,2-trifluoroethyl group, 2,2,3,3,3-pentafluoropropyl group, 3-cyanopropyl group, 4-cyanobutyl group, methoxyphenyl group, cyanophenyl group, N-methylpyrazolylmethyl group, thienylethyl group, propargyl group, allyl group, and 3-butenyl group is more preferable.

In the fluorine-containing ether compound represented by Formula (1), z represents 1 or 2. The fluorine-containing ether compound represented by Formula (1) has favorable hydrophobicity because z is 1 or 2, compared to a compound in which the number (z+1) of PFPE chains represented by R² is 2 or 3, and the number of PFPE chains represented by R² is 1. Therefore, even if the thickness is thin, it is possible to form a lubricating layer having a strong effect of inhibiting corrosion of the magnetic recording medium. In addition, since z is 1 or 2, the number of R³'s arranged between R²'s is 1 or 2. Therefore, the number of polar groups of —R²[—CH₂—R³—CH₂—R²]ₓ— in Formula (1) is appropriate, and for example, compared to when z is 0, it is possible to form a lubricating layer having favorable adhesion to the protective layer. In addition, for example, compared to when z is 3 or more, the fluorine-containing ether compound represented by Formula (1) can prevent interaction between the polar groups in the molecule and the polar groups of the fluorine-containing ether compound are unlikely to aggregate with each other.

### (PFPE Chain Represented by R²)

In the fluorine-containing ether compound represented by Formula (1), (z+1) R²'s are each independently a perfluoropolyether chain. When the lubricant containing the fluorine-containing ether compound of the present embodiment is applied onto the protective layer to form a lubricating layer, the PFPE chain represented by R² covers the surface of the protective layer, imparts lubricity to the lubricating layer, and reduces the frictional force between the magnetic head and the protective layer. The PFPE chain represented by R² is appropriately selected depending on the performance required for the lubricant containing the fluorine-containing ether compound and the like.

In the fluorine-containing ether compound represented by Formula (1), some or all of (z+1) R²'s may be the same as or different from each other. All of the (z+1) R²'s are preferably the same. This is because the coating of the fluorine-containing ether compound on the protective layer becomes uniform, and a lubricating layer having better adhesion is formed.

“Two or more R²'s among (z+1) R²'s are the same” means that, among (z+1) R²'s, two or more R²'s have the same repeating unit structure of the PFPE chain. The same R² include those having the same repeating unit structure but different average degrees of polymerization.

Examples of PFPE chains represented by R² include those composed of perfluoroalkylene oxide polymers or copolymers. Examples of perfluoroalkylene oxides include perfluoromethylene oxides, perfluoroethylene oxides, perfluoro-n-propylene oxides, perfluoroisopropylene oxides, and perfluorobutylene oxides.

Two or three R²'s in Formula (1) are each independently preferably a PFPE chain represented by the following Formula (9) derived from a perfluoroalkylene oxide polymer or copolymer.

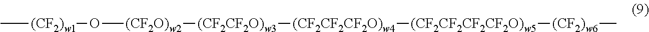

(in Formula (9), w2, w3, w4, and w5 indicate an average degree of polymerization and each independently represent 0 to 20; provided that all of w2, w3, w4, and w5 are not 0 at the same time, w1 and w6 are an average value representing the number of CF₂'s and each independently represent 1 to 3, and the arrangement order of repeating units (CF₂O), (CF₂CF₂O), (CF₂CF₂CF₂O), and (CF₂CF₂CF₂CF₂O) in Formula (9) is not particularly limited).

In Formula (9), w2, w3, w4, and w5 indicate an average degree of polymerization and each independently represent 0 to 20, and are preferably 0 to 15 and more preferably 0 to 10.

In Formula (9), w1 and w6 are an average value indicating the number of CF₂'s, and each independently represent 1 to 3. w1 and w6 are determined according to the structure of repeating units arranged at the ends of the chain structure in the PFPE chain represented by Formula (9).

In Formula (9), (CF₂O), (CF₂CF₂O), (CF₂CF₂CF₂O), and (CF₂CF₂CF₂CF₂O) are repeating units. The arrangement order of repeating units in Formula (9) is not particularly limited. In addition, the number of types of repeating units in Formula (9) is not particularly limited.

Two or three R²'s in Formula (1) are each independently preferably any one selected from among PFPE chains represented by the following Formulae (10-1) to (10-4).

When (z+1) R²'s in Formula (1) each are any one selected from among PFPE chains represented by Formulae (10-1) to (10-4), a fluorine-containing ether compound which can form a lubricating layer having favorable lubricity is obtained. In addition, when (z+1) R²'s each are any one selected from among PFPE chains represented by Formulae (10-1) to (10-4), the ratio of the number of oxygen atoms (the number of ether bonds (—O—)) to the number of carbon atoms in the PFPE chain is appropriate. Therefore, the fluorine-containing ether compound having an appropriate hardness is obtained. Therefore, the fluorine-containing ether compound applied onto the protective layer is unlikely to aggregate on the protective layer, and a thinner lubricating layer can be formed at a sufficient coating rate. In addition, since the fluorine-containing ether compound has appropriate flexibility, a lubricating layer having better pickup characteristics and spin-off characteristics can be formed.

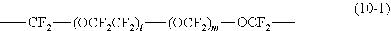

(in Formula (10-1), 1 and m indicate an average degree of polymerization, l represents 0.1 to 20, and m represents 0 to 20).

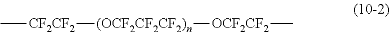

(in Formula (10-2), n indicates an average degree of polymerization and represents 0.1 to 15).

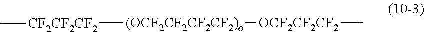

(in Formula (10-3), o indicates an average degree of polymerization and represents 0.1 to 10).

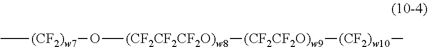

(in Formula (10-4), w8 and w9 indicate an average degree of polymerization and each independently represent 0.1 to 20. w7 and w10 are an average value representing the number of CF₂'s and each independently represent 1 to 2).

In Formula (10-1), the arrangement order of repeating units (OCF₂CF₂) and (OCF₂) is not particularly limited. In Formula (10-1), the number 1 of (OCF₂CF₂)'s and the number m of (OCF₂)'s may be the same as or different from each other. The PFPE chain represented by Formula (10-1) may be a polymer of (OCF₂CF₂). In addition, the PFPE chain represented by Formula (10-1) may be any of a random copolymer, a block copolymer, and an alternating copolymer composed of (OCF₂CF₂) and (OCF₂).

In Formulae (10-1) to (10-3), since 1 indicating an average degree of polymerization is 0.1 to 20, m is 0 to 20, n is 0.1 to 15, and o is 0.1 to 10, a fluorine-containing ether compound which can form a lubricating layer having favorable lubricity is obtained. In addition, in Formulae (10-1) to (10-3), when l and m indicating an average degree of polymerization are 20 or less, n is 15 or less, and o is 10 or less, this is preferable because the viscosity of the fluorine-containing ether compound does not become too high, and a lubricant containing this is easily applied. 1, m, n, and o indicating an average degree of polymerization are preferably 1 to 10, more preferably 1.5 to 8, and still more preferably 2 to 7 because a fluorine-containing ether compound which easily wets and spreads on the protective layer and allows a lubricating layer having a uniform film thickness to be obtained is obtained.

In Formula (10-4), the arrangement order of repeating units (CF₂CF₂CF₂O) and (CF₂CF₂O) is not particularly limited. In Formula (10-4), the number w8 of (CF₂CF₂CF₂O)'s and the number w9 of (CF₂CF₂O)'s, which indicate an average degree of polymerization, may be the same as or different from each other. Formula (10-4) may contain any of a random copolymer, a block copolymer, and an alternating copolymer composed of monomer units (CF₂CF₂CF₂O) and (CF₂CF₂O).

In Formula (10-4), w8 and w9 indicating an average degree of polymerization are each independently 0.1 to 20, preferably 1 to 15, and more preferably 1 to 10.

In Formula (10-4), w7 and w10 are an average value indicating the number of CF₂'s and each independently represent 1 to 2. w7 and w10 are determined according to the structure of repeating units arranged at the ends of the chain structure in the PFPE chain represented by Formula (10-4).

### (Divalent Linking Group Represented by R³)

In the fluorine-containing ether compound represented by Formula (1), one or two R³'s are the divalent linking group represented by Formula (5). In Formula (5), a dotted line bonded to the oxygen atom on the left side indicates a bond with a methylene group on the side of R¹, and a dotted line bonded to the oxygen atom on the right side indicates a bond with a methylene group on the side of R⁴. In Formula (1), when z is 1, R³ is arranged between two PFPE chains represented by R². When z is 2, two R³'s are arranged between R² on the side of R¹ and the center R² and between R² on the side of R⁴ and the center R².

R³ is a divalent linking group having a secondary hydroxy group. Therefore, R³ has favorable adhesion to the protective layer due to the secondary hydroxy group. Therefore, R³ prevents the PFPE chains represented by R² arranged at both terminals of R³ from moving too far away from the protective layer, and maintains an appropriate distance between the lubricating layer having favorable hydrophobicity derived from R² and the magnetic head. That is, R³ contributes to formation of a lubricating layer having a strong effect of inhibiting corrosion of the magnetic recording medium at a sufficient coating rate.

In Formula (5), y1 is an integer of 1 to 3, and y2 is an integer of 1 to 3. At least one of y1 and y2 is preferably 1. When at least one of y1 and y2 is 1, this is preferable because production becomes easy. In order to maintain the flexibility of the entire linking group, more preferably, y1 is 1 and y2 is 1.

In Formula (1), when z is 2, two R³'s may be the same as or different from each other. two R³'s are the same, the coating of the fluorine-containing ether compound on the protective layer becomes more uniform, and a lubricating layer having better adhesion can be formed. In addition, when two R³'s are the same, this is preferable because a fluorine-containing ether compound that is easy to produce is obtained.

“Two R³'s are the same” means that atoms contained in two R³'s are arranged symmetrically with respect to R² arranged in the center of a chain structure of a molecule. That is, when z is 2, the fluorine-containing ether compound represented by Formula (1) is preferably a fluorine-containing ether compound in which y1 and y2 in Formula (5) for two R³'s are values that are symmetrical with respect to R² arranged in the center of the chain structure. For example, when y1 in Formula (5) for R³ on the side of R¹ is 1 and y2 is 2, and y1 in Formula (5) for R³ on the side of R⁴ is 2 and y2 is 1, two R³'s are the same. In addition, for example, when y1 in Formula (5) for R³ on the side of R¹ is 2 and y2 is 1, and y1 in Formula (5) for R³ on the side of R⁴ is 1 and y2 is 2, two R³'s are the same.

In the fluorine-containing ether compound represented by Formula (1), since R¹—[B]-[A]- and —[C]-[D]-R⁴ are the same, atoms are preferably arranged symmetrically on both sides of the structure represented by —CH₂—R²[—CH₂—R³—CH₂—R²], —CH₂—. In this case, the production cost is low because production is easy.

In Formula (1), when z is 1, more preferably, R¹—[B]-[A]-and-[C]-[D]-R⁴ are the same, and two R²'s are the same. This is because a fluorine-containing ether compound is easily synthesized.

In Formula (1), when z is 2, more preferably, R¹—[B]-[A]-and-[C]-[D]-R⁴ are the same, three R²'s are the same. This is because a fluorine-containing ether compound is easily synthesized. In addition, when z is 2, atoms contained in two R³'s are preferably arranged symmetrically with respect to R² arranged in the center of a chain structure of a molecule. This is because a fluorine-containing ether compound is more easily synthesized.

Specifically, the fluorine-containing ether compound represented by Formula (1) is preferably a compound represented by the following Formulae (1A) to (1V), and (2A) to (2V).

In the compounds represented by the following Formulae (1A) to (1V), and (2A) to (2V), Rf₁ and Rf₂ representing the PFPE chain have the following structures. That is, Rf₁ is the PFPE chain represented by Formula (10-1), and Rf₂ is the PFPE chain represented by Formula (10-2). Here, since 1 and m in Rf₁ and n in Rf₂, which represent the PFPE chain in Formulae (1A) to (1V), and (2A) to (2V), are values indicating an average degree of polymerization, they are not necessarily an integer.

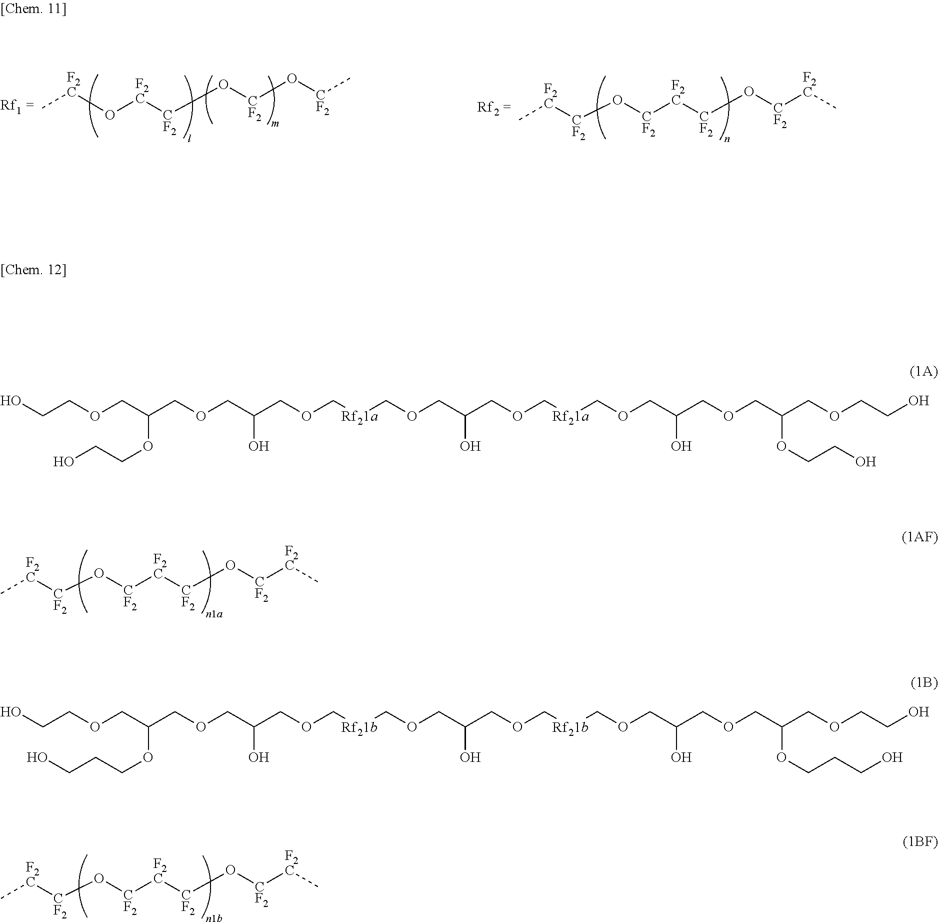

(in Formula (1A), Rf₂1a is represented by Formula (1AF), in Rf₂1a, n1a indicates an average degree of polymerization and represents 0.1 to 15, and in Formula (1A), in two Rf₂1a's, n1a's may be the same as or different from each other).
(in Formula (1B), Rf₂1b is represented by Formula (1BF), in Rf₂1b, n1b indicates an average degree of polymerization and represents 0.1 to 15, and in Formula (1B), in two Rf₂1b's, n1b's may be the same as or different from each other).

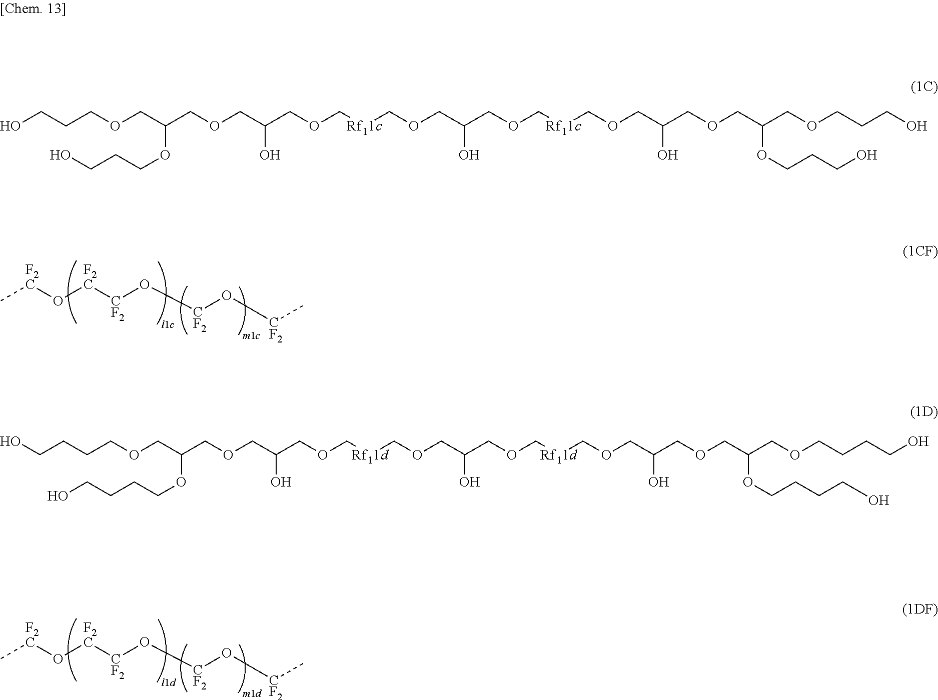

(in Formula (1C), Rf₁1c is represented by Formula (1CF), in Rf₁1c, l1c and m1c indicate an average degree of polymerization, l1c represents 0.1 to 20, and m1c represents 0 to 20, and in Formula (1C), in two Rf₁1c's, l1c's and m1c's may be the same as or different from each other).
(in Formula (1D), Rf₁1d is represented by Formula (1DF), in Rf₁1d, l1d and m1d indicate an average degree of polymerization, l1d represents 0.1 to 20, and m1d represents 0 to 20, and in Formula (1D), in two Rf₁1d's, l1d's and m1d's may be the same as or different from each other).

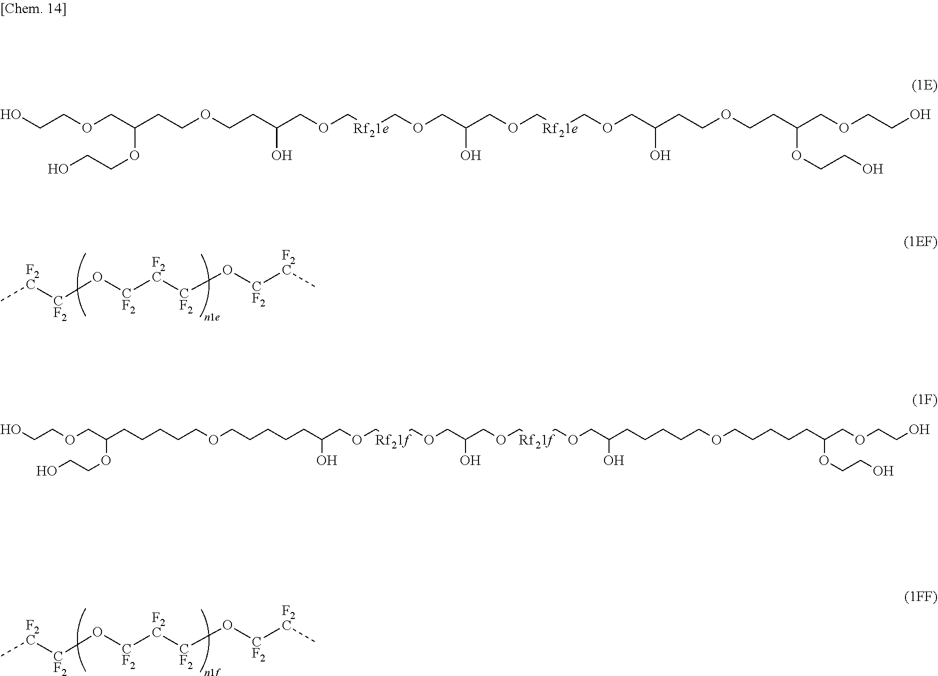

(in Formula (1E), Rf₂1e is represented by Formula (1EF), in Rf₂1e, n1e indicates an average degree of polymerization and represents 0.1 to 15, and in Formula (1E), in two Rf₂1e's, ne's may be the same as or different from each other).
(in Formula (1F), Rf₂1f is represented by Formula (1FF), in Rf₂1f, n1f indicates an average degree of polymerization and represents 0.1 to 15, and in Formula (1F), in two Rf₂1f's, n1f's may be the same as or different from each other).

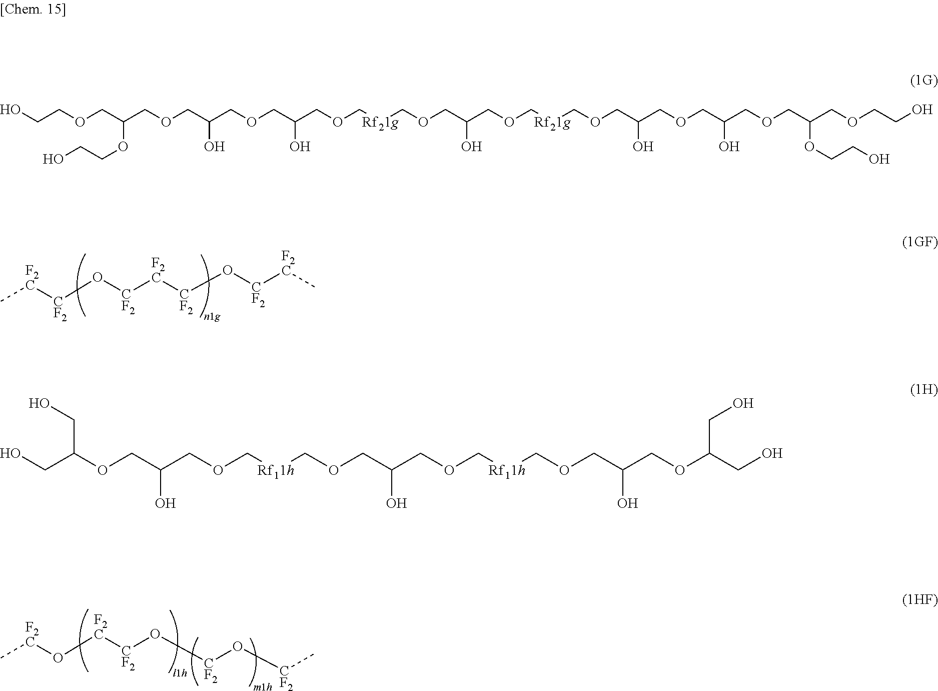

(in Formula (1G), Rf₂1g is represented by Formula (1GF), in Rf₂1g, n1g indicates an average degree of polymerization and represents 0.1 to 15, and in Formula (1G), in two Rf₂1g's, n1g's may be the same as or different from each other).
(in Formula (1H), Rf₁1h is represented by Formula (1HF), in Rf₁1h, l1h and m1h indicate an average degree of polymerization, l1h represents 0.1 to 20, and m1h represents 0 to 20, and in Formula (1H), in two Rf₁1h's, l1h's and m1h's may be the same as or different from each other).

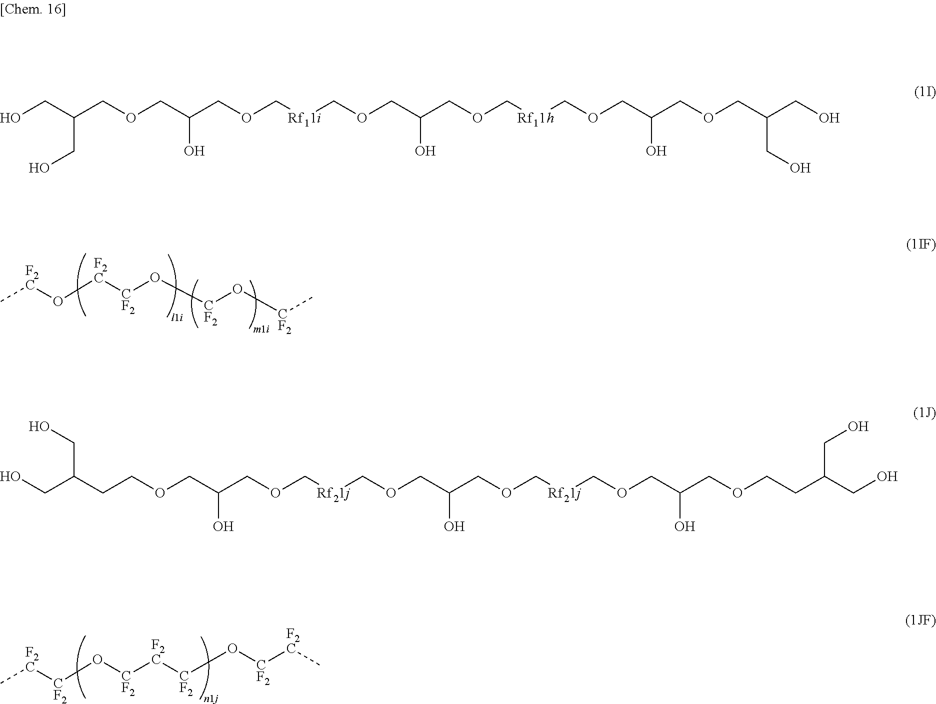

(in Formula (11), Rf₁1i is represented by Formula (1IF), in Rf₁1i, l1i and m1i indicate an average degree of polymerization, l1i represents 0.1 to 20, and m1i represents 0 to 20, and in Formula (11), in two Rf₁1i's, l1i's and m1i's may be the same as or different from each other).
(in Formula (1J), Rf₂1j is represented by Formula (1JF), in Rf₂1j, n1j indicates an average degree of polymerization and represents 0.1 to 15, and in Formula (1J), in two Rf₂1j's, n1j's may be the same as or different from each other).

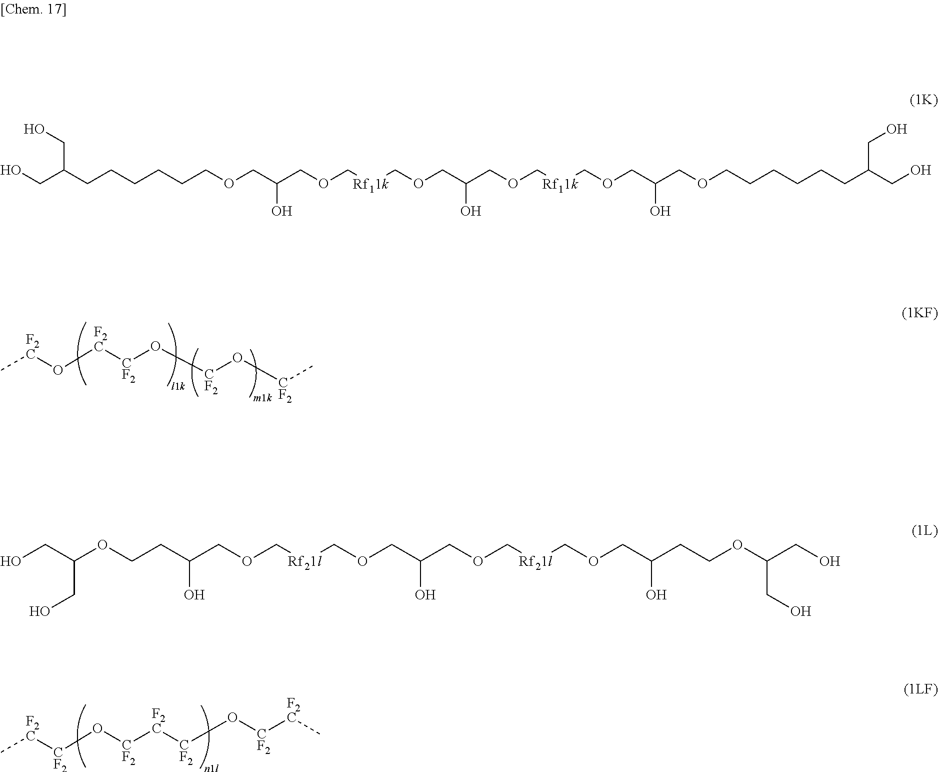

(in Formula (1K), Rf₁1k is represented by Formula (1KF), in Rf₁1k, l1k and m1k indicate an average degree of polymerization, l1k represents 0.1 to 20, and m1k represents 0 to 20, and in Formula (1K), in two Rf₁1k's, l1k's and m1k's may be the same as or different from each other).
(in Formula (1L), Rf₂1l is represented by Formula (1LF), in Rf₂1l, n1l indicates an average degree of polymerization and represents 0.1 to 15, and in Formula (1L), in two Rf₂1l's, n1l's may be the same as or different from each other).

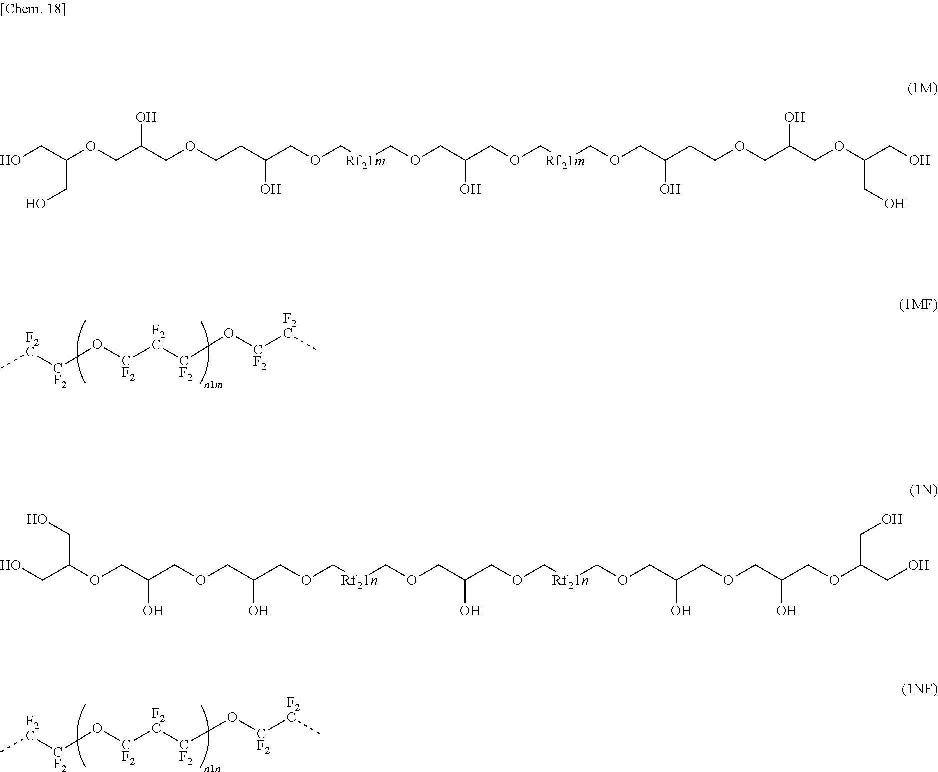

(in Formula (1M), Rf₂1m is represented by Formula (1MF), in Rf₂1m, nlm indicates an average degree of polymerization and represents 0.1 to 15, and in Formula (1M), in two Rf₂1m's, n1m's may be the same as or different from each other).
(in Formula (1N), Rf₂1n is represented by Formula (1NF), in Rf₂1n, nin indicates an average degree of polymerization and represents 0.1 to 15, and in Formula (1N), in two Rf₂1n's, n1n's may be the same as or different from each other).

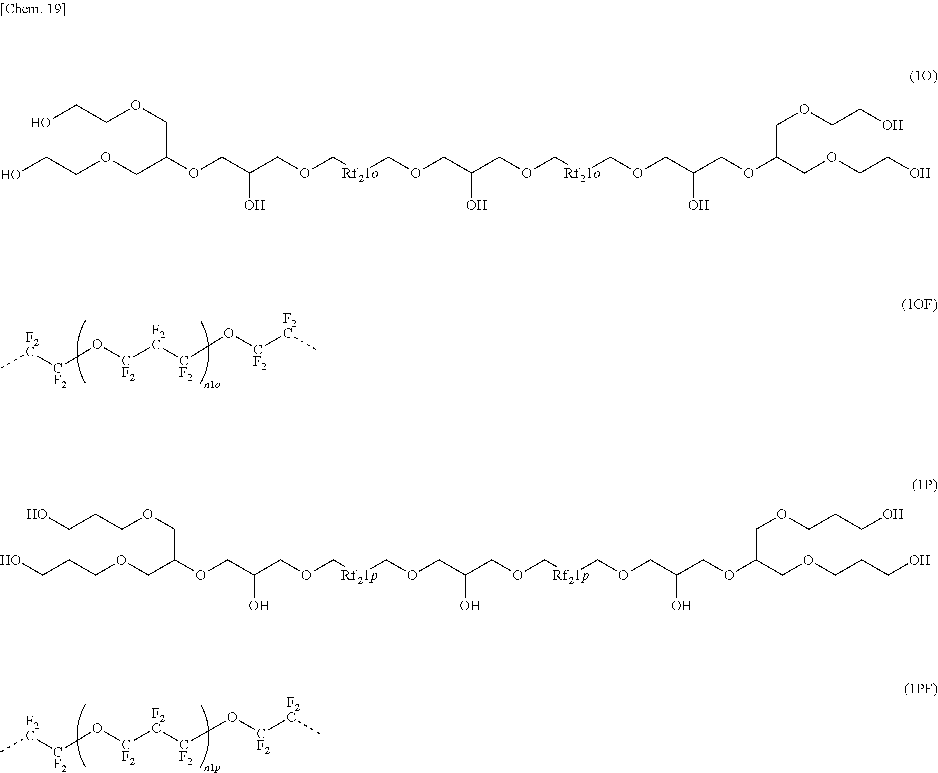

(in Formula (10), Rf₂1o is represented by Formula (1° F.), in Rf₂1o, n1o indicates an average degree of polymerization and represents 0.1 to 15, and in Formula (10), in two Rf₂1o's, n1o's may be the same as or different from each other).
(in Formula (1P), Rf₂1p is represented by Formula (1 PF), in Rf₂1p, n1p indicates an average degree of polymerization and represents 0.1 to 15, and in Formula (1P), in two Rf₂1p's, n1p's may be the same as or different from each other).

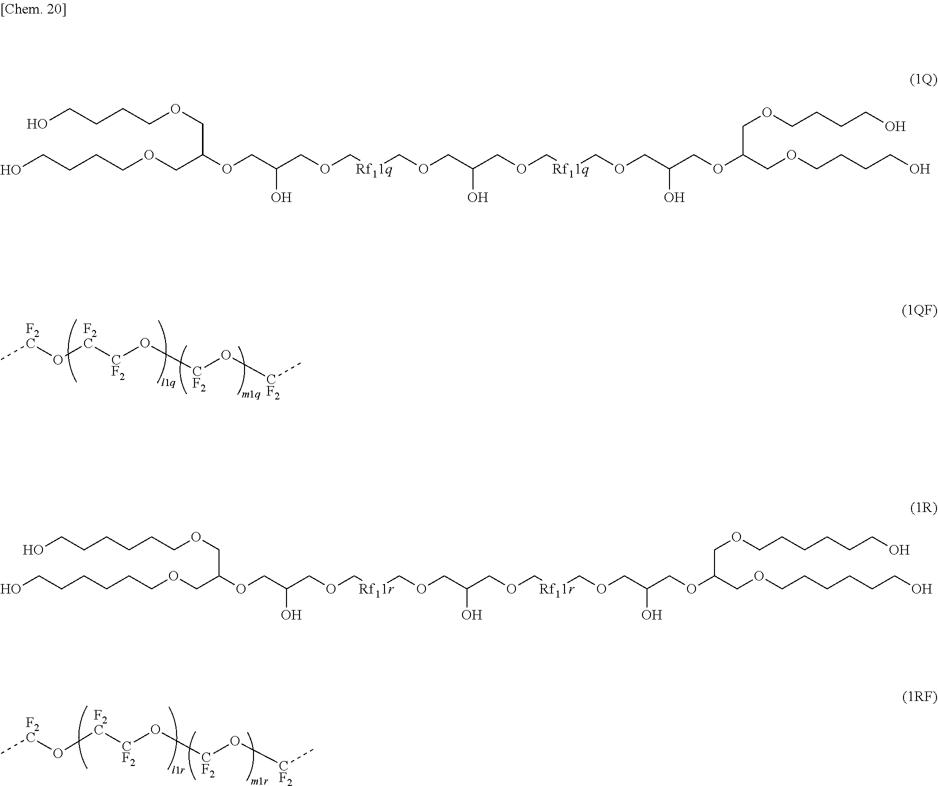

(in Formula (1Q), Rf₁1q is represented by Formula (1QF), in Rf₁1q, l1q and m1q indicate an average degree of polymerization, l1q represents 0.1 to 20, and m1q represents 0 to 20, and in Formula (1Q), in two Rf₁1q's, l1q's and m1q's may be the same as or different from each other).
(in Formula (1R), Rf₁1r is represented by Formula (1RF), in Rf₁1r, l1r and m1r indicate an average degree of polymerization, l1r represents 0.1 to 20, and m1r represents 0 to 20, and in Formula (1R), in two Rf₁1r's, l1r's and m1r's may be the same as or different from each other).

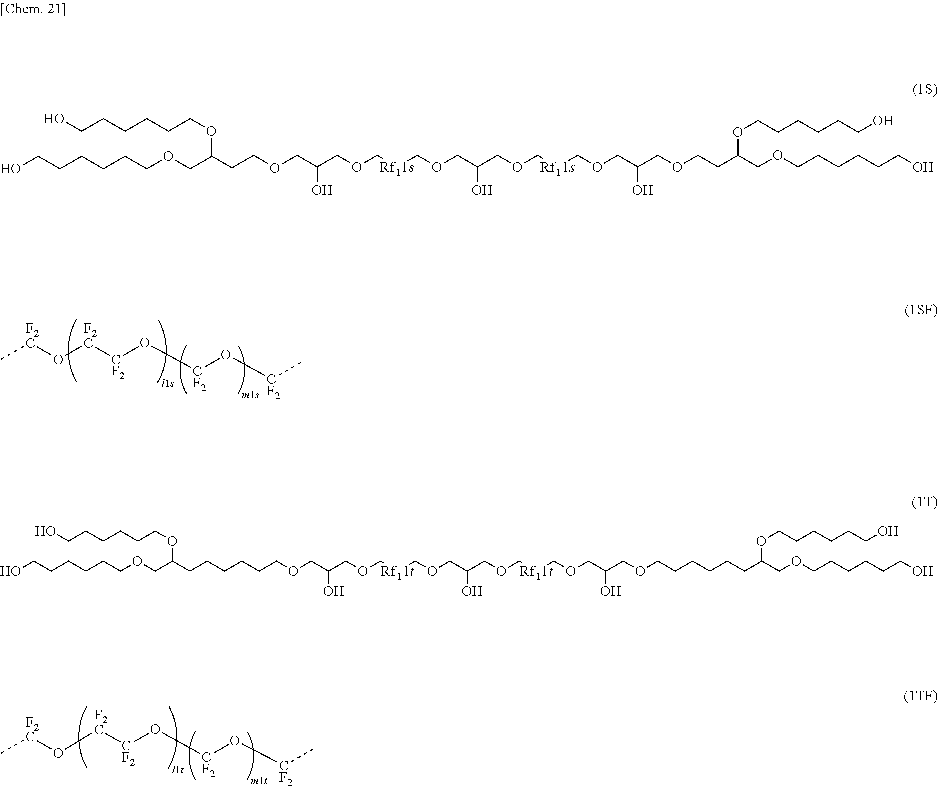

(in Formula (1S), Rf₁1s is represented by Formula (1SF), in Rf₁1s, l1s and m1s indicate an average degree of polymerization, l1s represents 0.1 to 20, and m1s represents 0 to 20, and in Formula (1S), in two Rf₁1s's, l1s's and m1s's may be the same as or different from each other).
(in Formula (1T), Rf₁ It is represented by Formula (1TF), in Rf₁1t, l1t and m1t indicate an average degree of polymerization, l1t represents 0.1 to 20, and m1t represents 0 to 20, and in Formula (1T), in two Rf₁1t's, l1t's and m1t's may be the same as or different from each other).

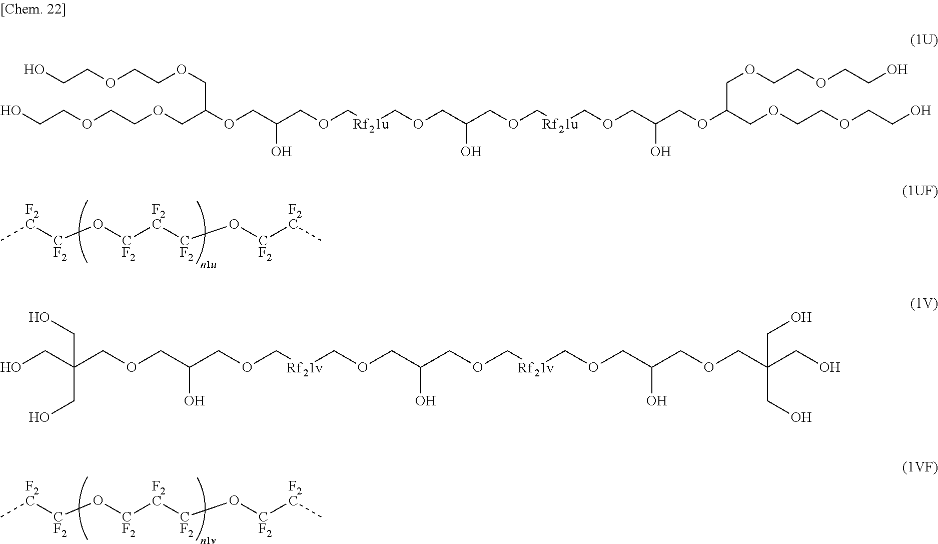

(in Formula (1U), Rf₂1u is represented by Formula (1UF), in Rf₂1u, n1u indicates an average degree of polymerization and represents 0.1 to 15, and in Formula (1U), in two Rf₂1u's, n1u's may be the same as or different from each other).
(in Formula (1V), Rf₂1v is represented by Formula (1VF), in Rf₂1v, n1v indicates an average degree of polymerization and represents 0.1 to 15, and in Formula (1V), in two Rf₂1v's, n1v's may be the same as or different from each other).

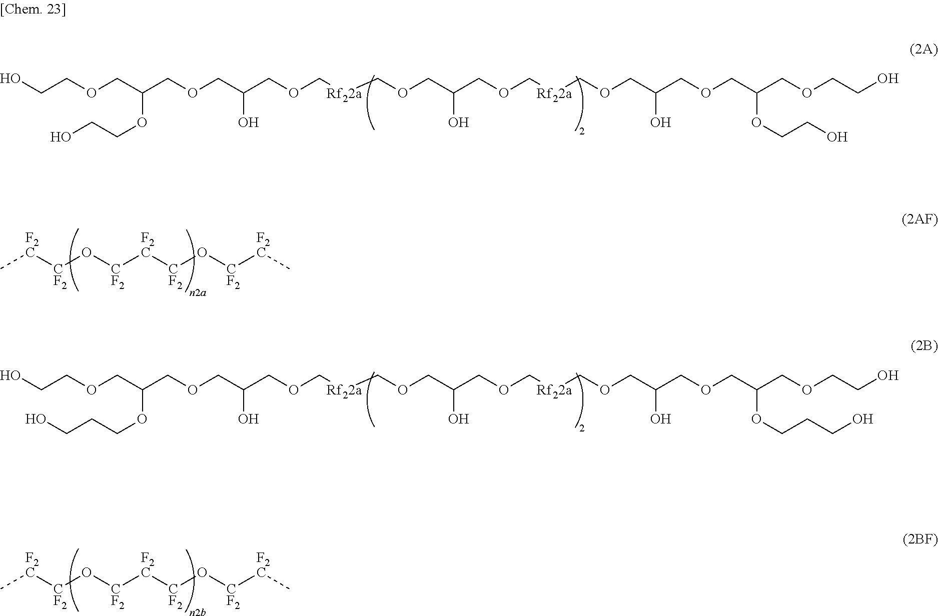

(in Formula (2A), Rf₂2a is represented by Formula (2AF), in Rf₂2a, n2a indicates an average degree of polymerization and represents 0.1 to 15, and in Formula (2A), in three Rf₂2a's, n2a's may be different from each other, or some or all of them may be the same).
(in Formula (2B), Rf₂2b is represented by Formula (2BF), in Rf₂2b, n2b indicates an average degree of polymerization and represents 0.1 to 15, and in Formula (2B), in three Rf₂2b's, n2b's may be different from each other, or some or all of them may be the same).

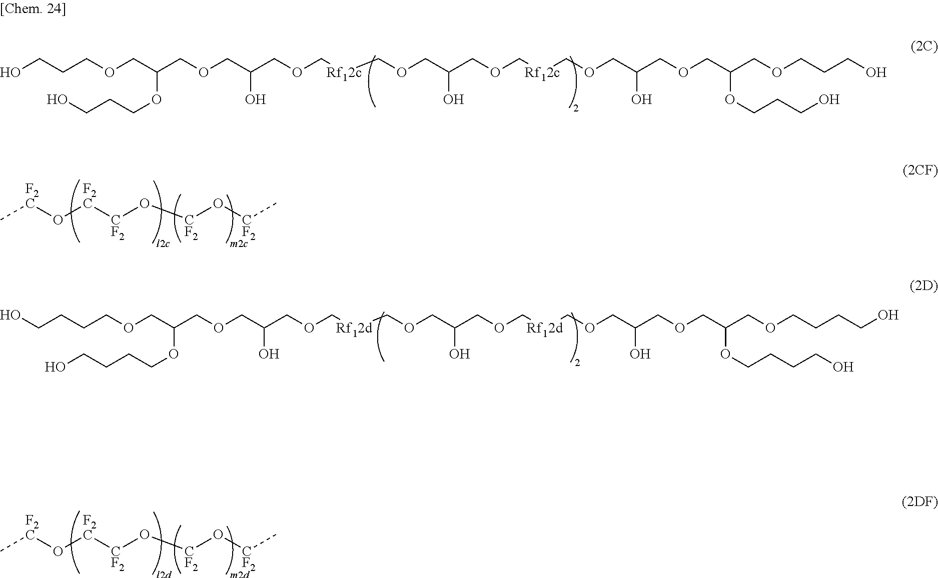

(in Formula (2C), Rf₁2c is represented by Formula (2CF), in Rf₁2c, 12c and m2c indicate an average degree of polymerization, l2c represents 0.1 to 20, and m2c represents 0 to 20, and in Formula (2C), in three Rf₁2c's, l2c's and m2c's may be different from each other, or some or all of them may be the same).
(in Formula (2D), Rf₁2d is represented by Formula (2DF), in Rf₁2d, 12d and m2d indicate an average degree of polymerization, l2d represents 0.1 to 20, and m2d represents 0 to 20, and in Formula (2D), in three Rf₁2d's, l2d's and m2d's may be different from each other, or some or all of them may be the same).

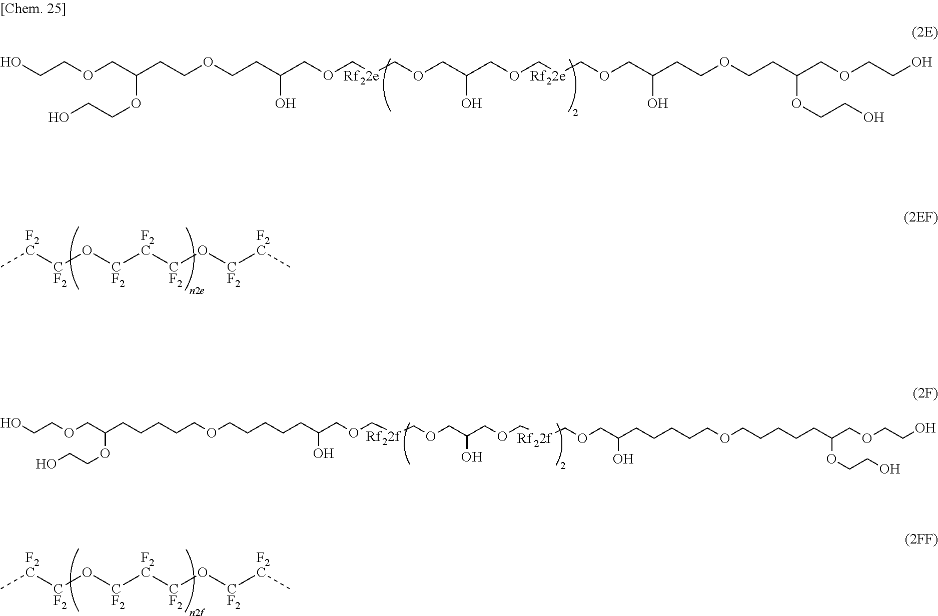

(in Formula (2E), Rf₂2e is represented by Formula (2EF), in Rf₂2e, n2e indicates an average degree of polymerization and represents 0.1 to 15, and in Formula (2E), in three Rf₂2e's, n2e's may be different from each other, or some or all of them may be the same).
(in Formula (2F), Rf₂2f is represented by Formula (2FF), in Rf₂2f, n2f indicates an average degree of polymerization and represents 0.1 to 15, and in Formula (2F), in three Rf₂2f's, n2f's may be different from each other, or some or all of them may be the same).

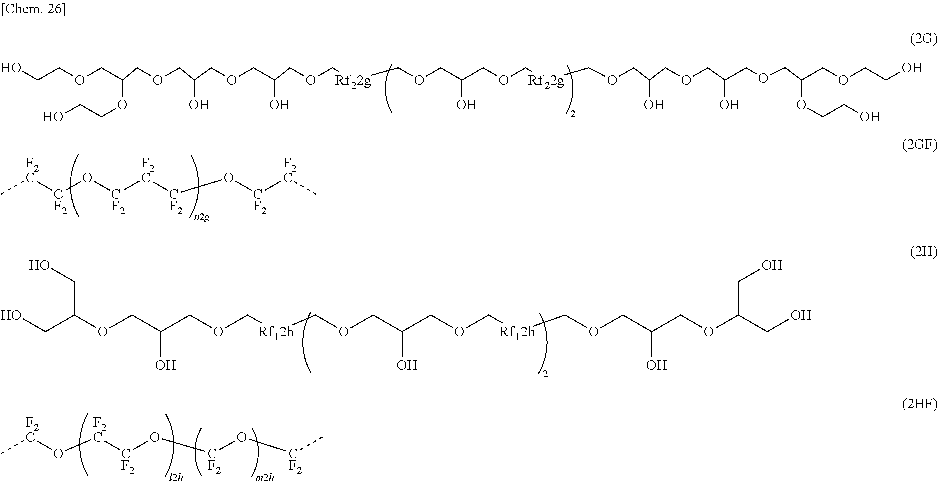

(in Formula (2G), Rf₂2g is represented by Formula (2GF), in Rf₂2g, n2g indicates an average degree of polymerization and represents 0.1 to 15, and in Formula (2G), in three Rf₂2g's, n2g's may be different from each other, or some or all of them may be the same).
(in Formula (2H), Rf₁2h is represented by Formula (2HF), in Rf₁2h, l1h and m2h indicate an average degree of polymerization, l2h represents 0.1 to 20, and m2h represents 0 to 20, and in Formula (2H), in three Rf₁2h's, l2h's and m2h's may be different from each other, or some or all of them may be the same).

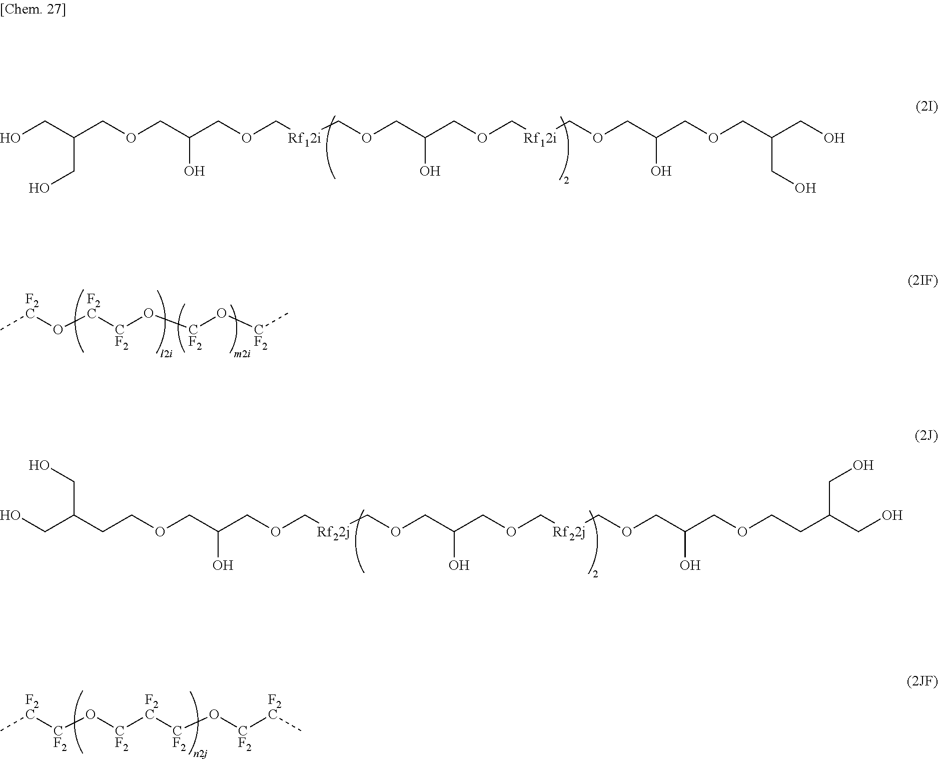

(in Formula (21), Rf₁2i is represented by Formula (2IF), in Rf₁2i, 12i and m2i indicate an average degree of polymerization, l2i represents 0.1 to 20, and m2i represents 0 to 20, and in Formula (21), in three Rf₁2i's, l2i's and m2i's may be different from each other, or some or all of them may be the same).
(in Formula (2J), Rf₂2j is represented by Formula (2JF), in Rf₂2j, n2j indicates an average degree of polymerization and represents 0.1 to 15, and in Formula (2J), in three Rf₂2j's, n2j's may be different from each other, or some or all of them may be the same).

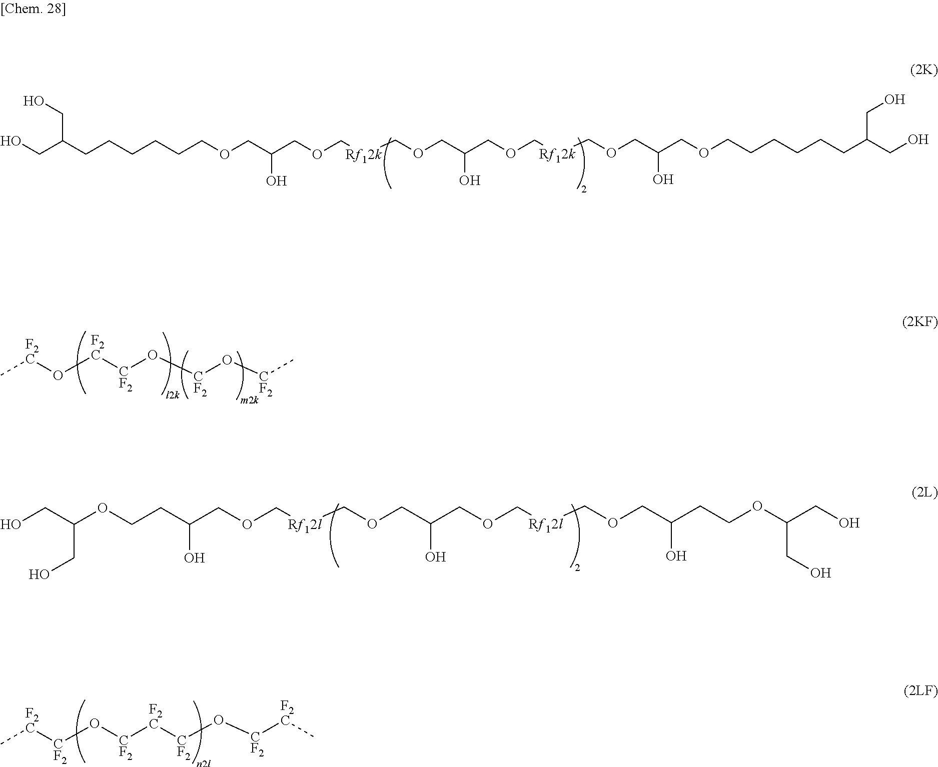

(in Formula (2K), Rf₁2k is represented by Formula (2KF), in Rf₁2k, l2k and m2k indicate an average degree of polymerization, l2k represents 0.1 to 20, and m2k represents 0 to 20, and in Formula (2K), in three Rf₁2k's, l2k's and m2k's may be different from each other, or some or all of them may be the same).
(in Formula (2L), Rf₂21 is represented by Formula (2LF), in Rf₂21, n21 indicates an average degree of polymerization and represents 0.1 to 15, and in Formula (2L), in three Rf₂21's, n2l's may be different from each other, or some or all of them may be the same).

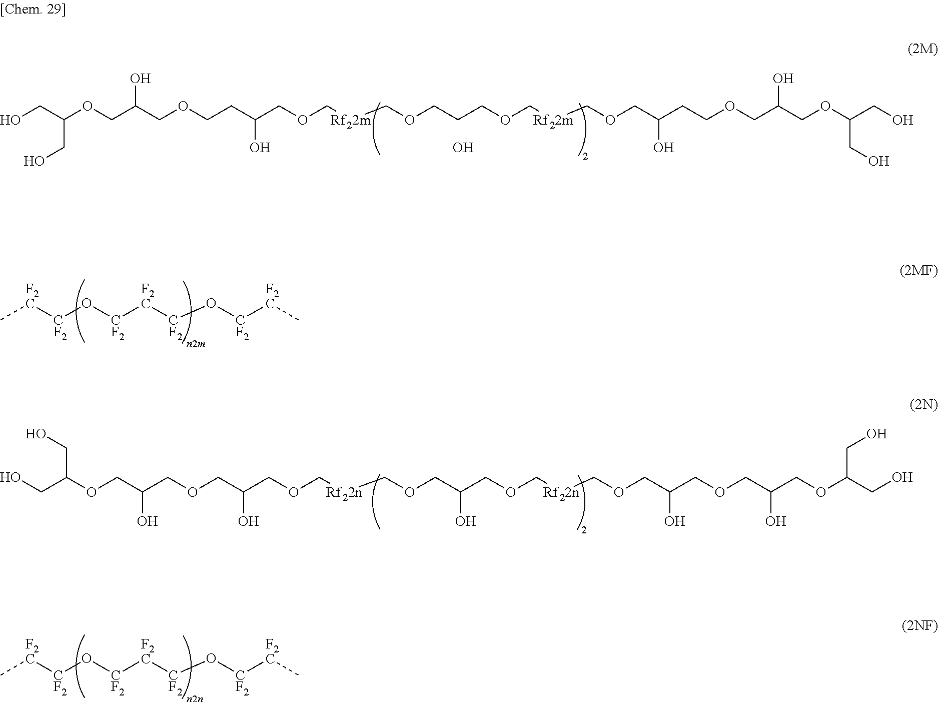

(in Formula (2M), Rf₂2m is represented by Formula (2MF), in Rf₂2m, n2m indicates an average degree of polymerization and represents 0.1 to 15, and in Formula (2M), in three Rf₂2m's, n2m's may be different from each other, or some or all of them may be the same).
(in Formula (2N), Rf₂2n is represented by Formula (2NF), in Rf₂2n, n2n indicates an average degree of polymerization and represents 0.1 to 15, and in Formula (2N), in three Rf₂2n's, n2n's may be different from each other, or some or all of them may be the same).

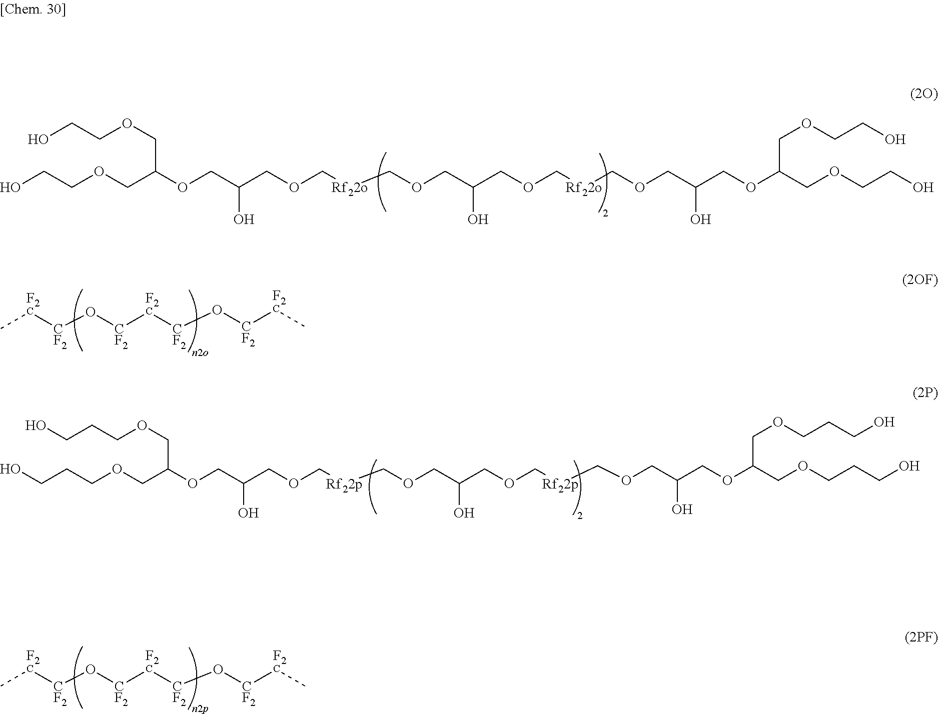

(in Formula (20), Rf₂20 is represented by Formula (2° F.), and in Rf₂20, n20 indicates an average degree of polymerization and represents 0.1 to 15, and in Formula (20), in three Rf₂20's, n2o's may be different from each other, or some or all of them may be the same).
(in Formula (2P), Rf₂2p is represented by Formula (2 PF), and in Rf₂2p, n2p indicates an average degree of polymerization and represents 0.1 to 15, and in Formula (2P), in three Rf₂2p's, n2p's may be different from each other, or some or all of them may be the same).

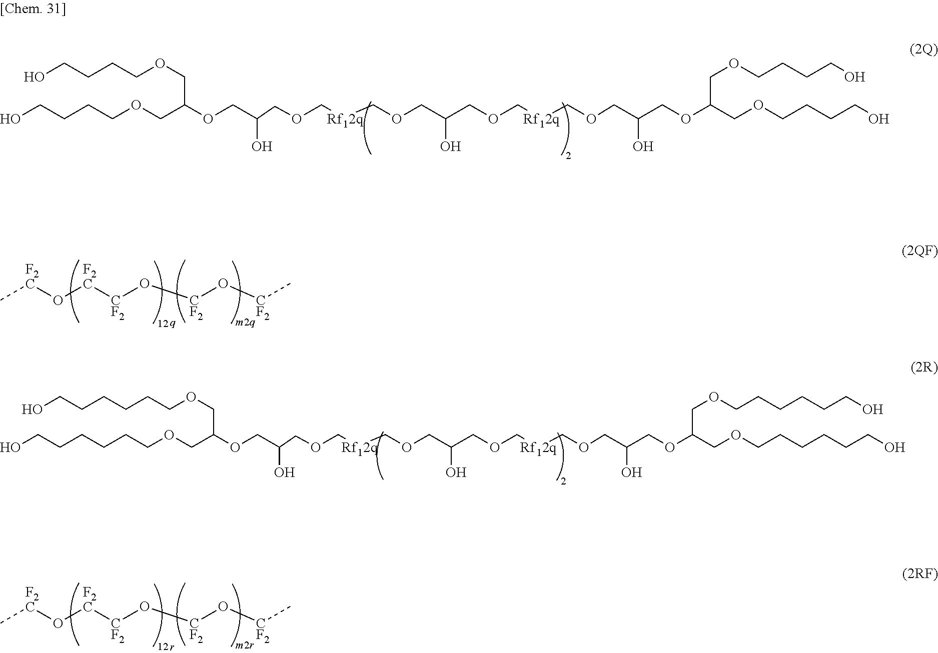

(in Formula (2Q), Rf₁2q is represented by Formula (2QF), in Rf₁2q, 12q and m2q indicate an average degree of polymerization, l2q represents 0.1 to 20, and m2q represents 0 to 20, and in Formula (2Q), in three Rf₁2q's, l2q's and m2q's may be different from each other, or some or all of them may be the same).
(in Formula (2R), Rf₁2r is represented by Formula (2RF), in Rf₁2r, 12r and m2r indicate an average degree of polymerization, l2r represents 0.1 to 20, and m2r represents 0 to 20, and in Formula (2R), in three Rf₁2r's, l2r's and m2r's may be different from each other, or some or all of them may be the same).

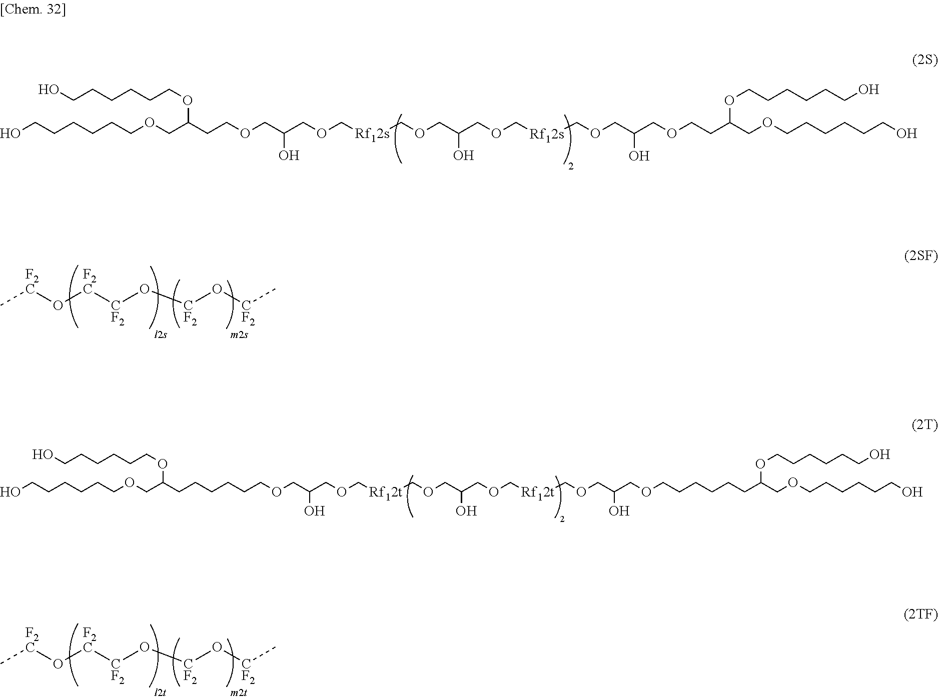

(in Formula (2S), Rf₁2s is represented by Formula (2SF), in Rf₁2s, l2s and m2s indicate an average degree of polymerization, l2s represents 0.1 to 20, and m2s represents 0 to 20, and in Formula (2S), in three Rf₁2s's, l2s's and m2s's may be different from each other, or some or all of them may be the same).
(in Formula (2T), Rf₁2t is represented by Formula (2TF), in Rf₁2t, l2t and m2t indicate an average degree of polymerization, l2t represents 0.1 to 20, and m2t represents 0 to 20, and in Formula (2T), in three Rf₁2t's, l2t's and m2t's may be different from each other, or some or all of them may be the same).

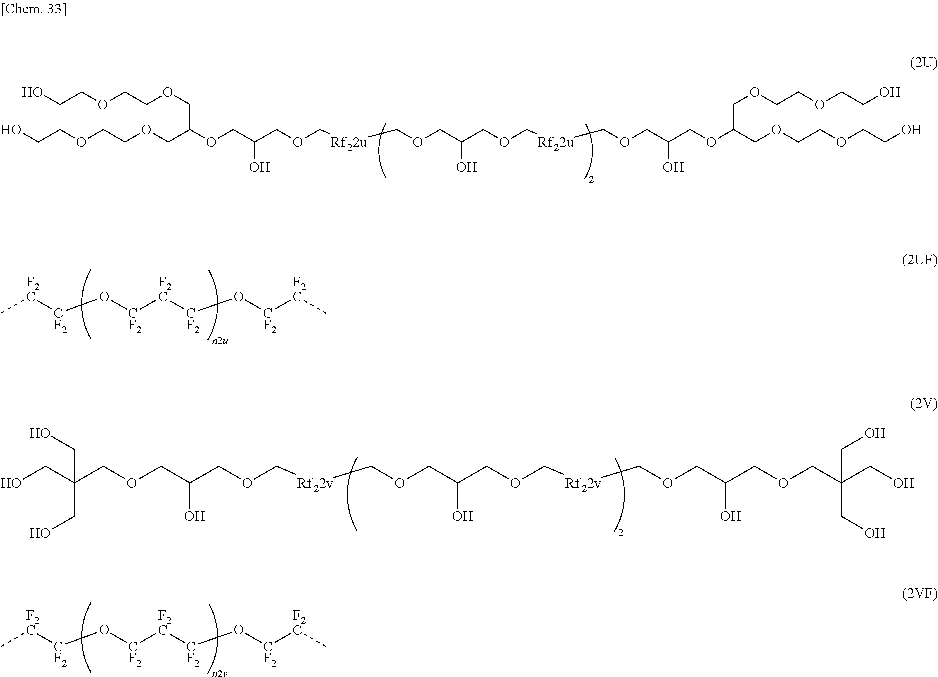

(in Formula (2U), Rf₂2u is represented by Formula (2UF), in Rf₂2u, n2u indicates an average degree of polymerization and represents 0.1 to 15, and in Formula (2U), in three Rf₂2u's, n2u's may be different from each other, or some or all of them may be the same).
(in Formula (2V), Rf₂2v is represented by Formula (2VF), in Rf₂2v, n2v indicates an average degree of polymerization and represents 0.1 to 15, and in Formula (2V), in three Rf₂2v's, n2v's may be different from each other, or some or all of them may be the same).

The fluorine-containing ether compound represented by Formula (1) is preferably a compound represented by the following Formulae (4A) to (40), and (5A) to (50).

In the compounds represented by the following Formulae (4A) to (40), and (5A) to (50), Rf₁ representing the PFPE chain is the PFPE chain represented by Formula (10-1), and Rf₂ is the PFPE chain represented by Formula (10-2). Here, since l and m in Rf₁ and n in Rf₂, which represent the PFPE chain in Formulae (4A) to (40), and (5A) to (50), are values indicating an average degree of polymerization, they are not necessarily an integer.

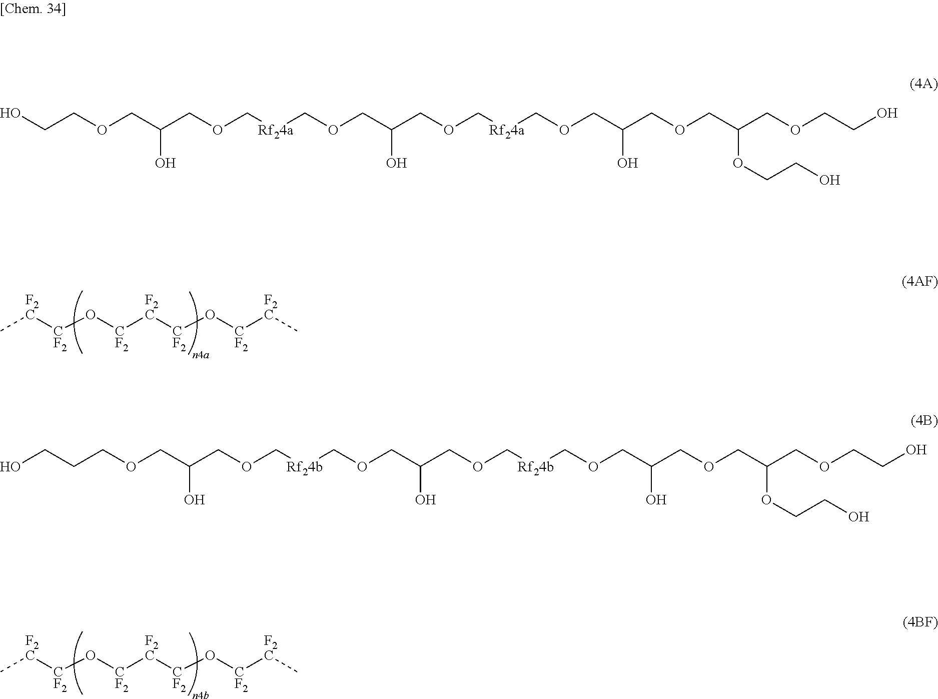

(in Formula (4A), Rf₂4a is represented by Formula (4AF), in Rf₂4a, n4a indicates an average degree of polymerization and represents 0.1 to 15, and in Formula (4A), in two Rf₂4a's, n4a's may be the same as or different from each other).
(in Formula (4B), Rf₂4b is represented by Formula (4BF), in Rf₂4b, n4b indicates an average degree of polymerization and represents 0.1 to 15, and in Formula (4B), in two Rf₂4b's, n4b's may be the same as or different from each other).

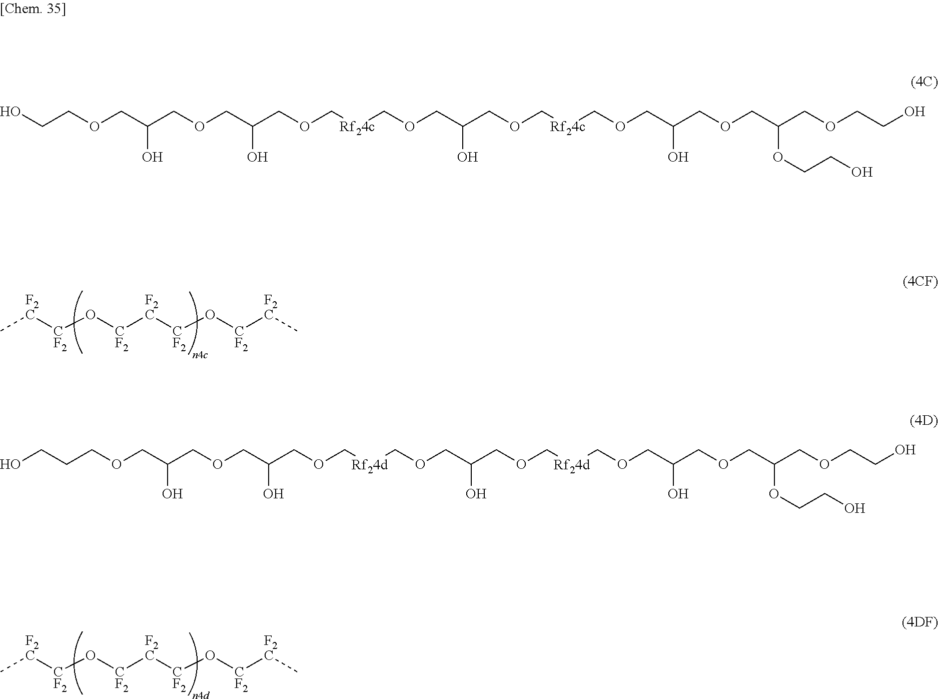

(in Formula (4C), Rf₂4c is represented by Formula (4CF), in Rf₂4c, n4c indicates an average degree of polymerization and represents 0.1 to 15, and in Formula (4C), in two Rf₂4c's, n4c's may be the same as or different from each other).
(in Formula (4D), Rf₂4d is represented by Formula (4DF), in Rf₂4d, n4d indicates an average degree of polymerization and represents 0.1 to 15, and in Formula (4D), in two Rf₂4d's, n4d's may be the same as or different from each other).

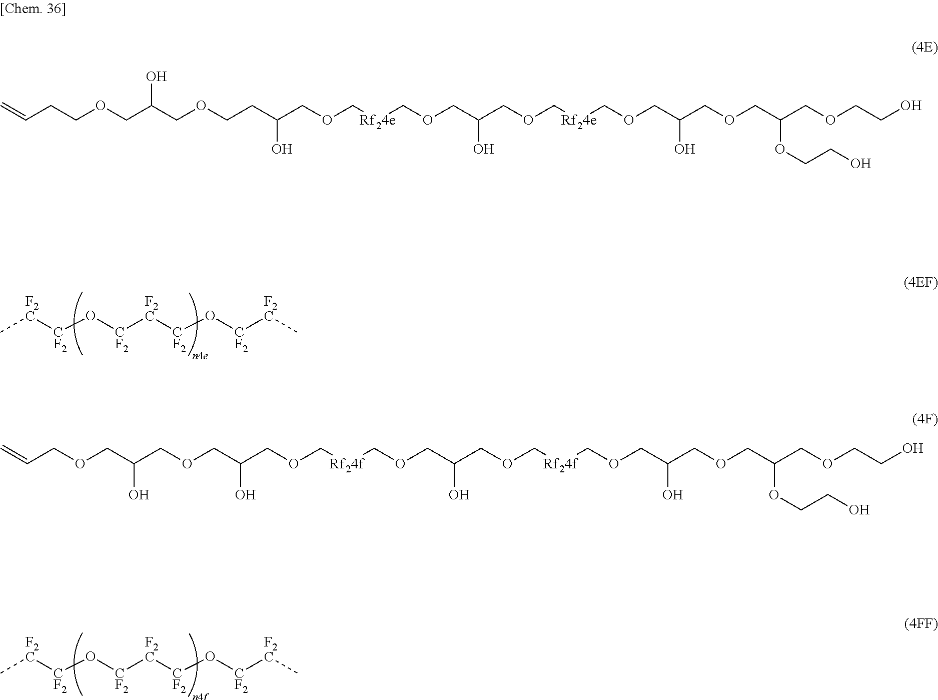

(in Formula (4E), Rf₂4e is represented by Formula (4EF), in Rf₂4e, n4e indicates an average degree of polymerization and represents 0.1 to 15, and in Formula (4E), in two Rf₂4e's, n4e's may be the same as or different from each other).
(in Formula (4F), Rf₂4f is represented by Formula (4FF), in Rf₂4f, n4f indicates an average degree of polymerization and represents 0.1 to 15, and in Formula (4F), in two Rf₂4f's, n4f's may be the same as or different from each other).

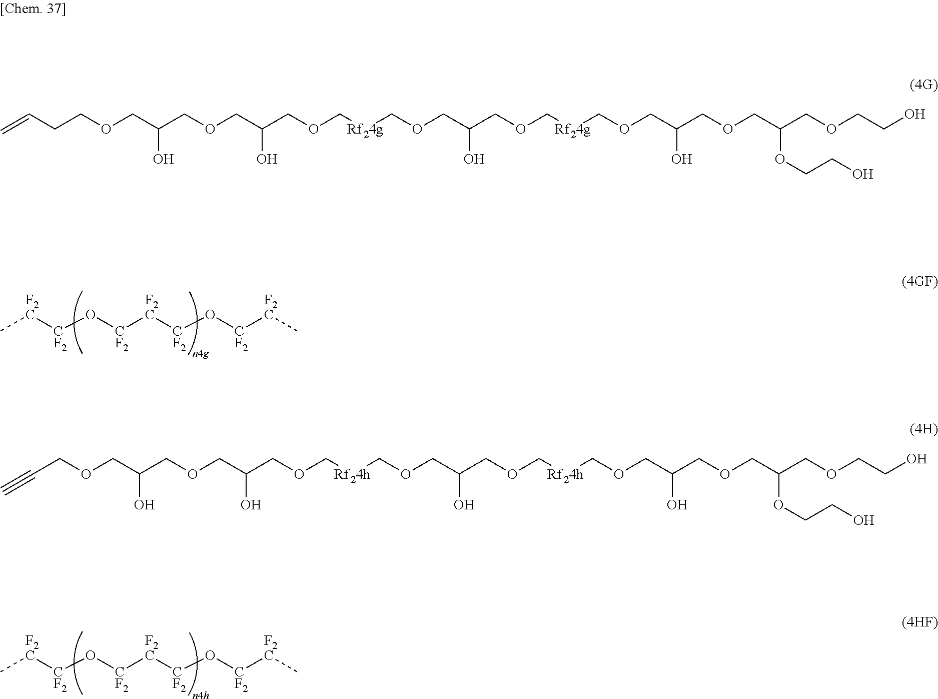

(in Formula (4G), Rf₂4g is represented by Formula (4GF), in Rf₂4g, n4g indicates an average degree of polymerization and represents 0.1 to 15, and in Formula (4G), in two Rf₂4g's, n4g's may be the same as or different from each other).
(in Formula (4H), Rf₂4h is represented by Formula (4HF), in Rf₂4h, n4h indicates an average degree of polymerization and represents 0.1 to 15, and in Formula (4H), in two Rf₂4h's, n4h's may be the same as or different from each other).

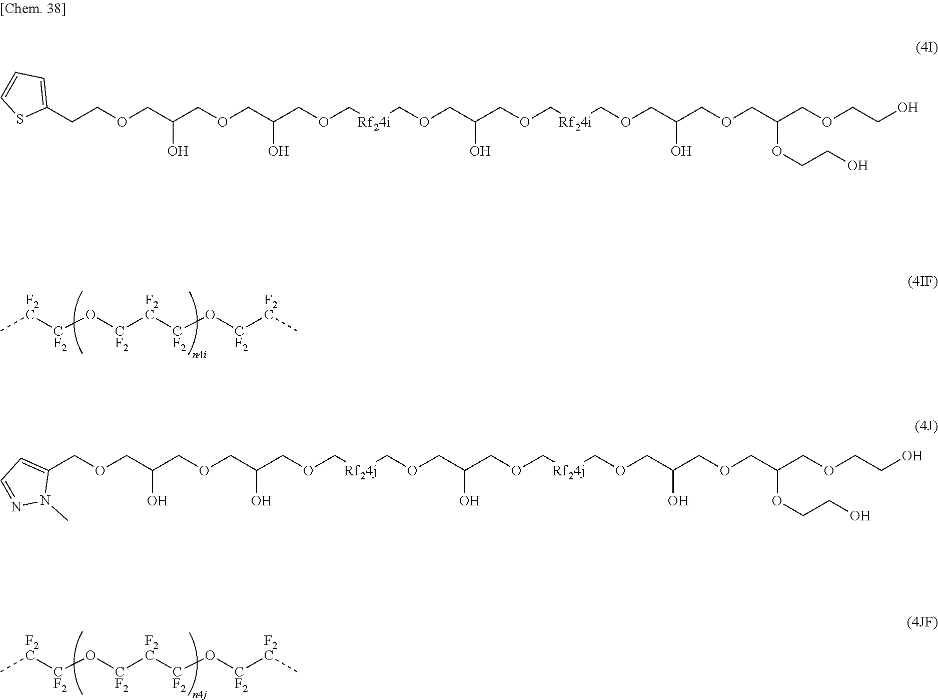

(in Formula (41), Rf₂4i is represented by Formula (4IF), in Rf₂4i, n4i indicates an average degree of polymerization and represents 0.1 to 15, and in Formula (41), in two Rf₂4i's, n4i's may be the same as or different from each other).
(in Formula (4J), Rf₂4j is represented by Formula (4JF), in Rf₂4j, n4j indicates an average degree of polymerization and represents 0.1 to 15, and in Formula (4J), in two Rf₂4j's, n4j's may be the same as or different from each other).

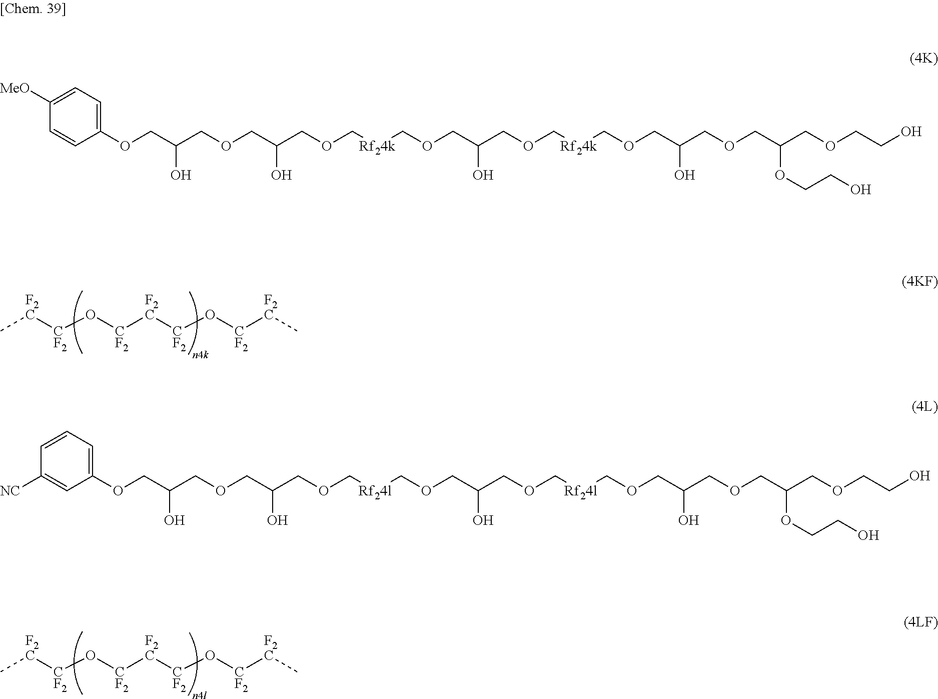

(in Formula (4K), Rf₂4k is represented by Formula (4KF), Me represents a methyl group, in Rf₂4k, n4k indicates an average degree of polymerization and represents 0.1 to 15, and in Formula (4K), in two Rf₂4k's, n4k's may be the same as or different from each other).
(in Formula (4L), Rf₂41 is represented by Formula (4LF), in Rf₂41, n41 indicates an average degree of polymerization and represents 0.1 to 15, and in Formula (4L), in two Rf₂41's, n4l's may be the same as or different from each other).

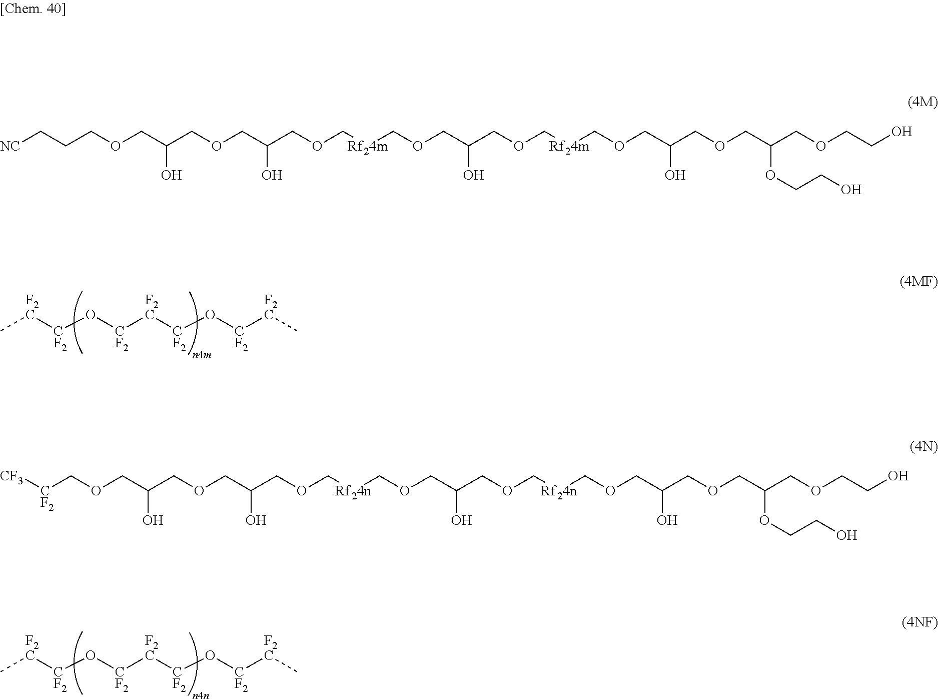

(in Formula (4M), Rf₂4m is represented by Formula (4MF), in Rf₂4m, n4m indicates an average degree of polymerization and represents 0.1 to 15, and in Formula (4M), in two Rf₂4m's, n4m's may be the same as or different from each other).
(in Formula (4N), Rf₂4n is represented by Formula (4NF), in Rf₂4n, n4n indicates an average degree of polymerization and represents 0.1 to 15, and in Formula (4N), in two Rf₂4n's, n4n's may be the same as or different from each other).

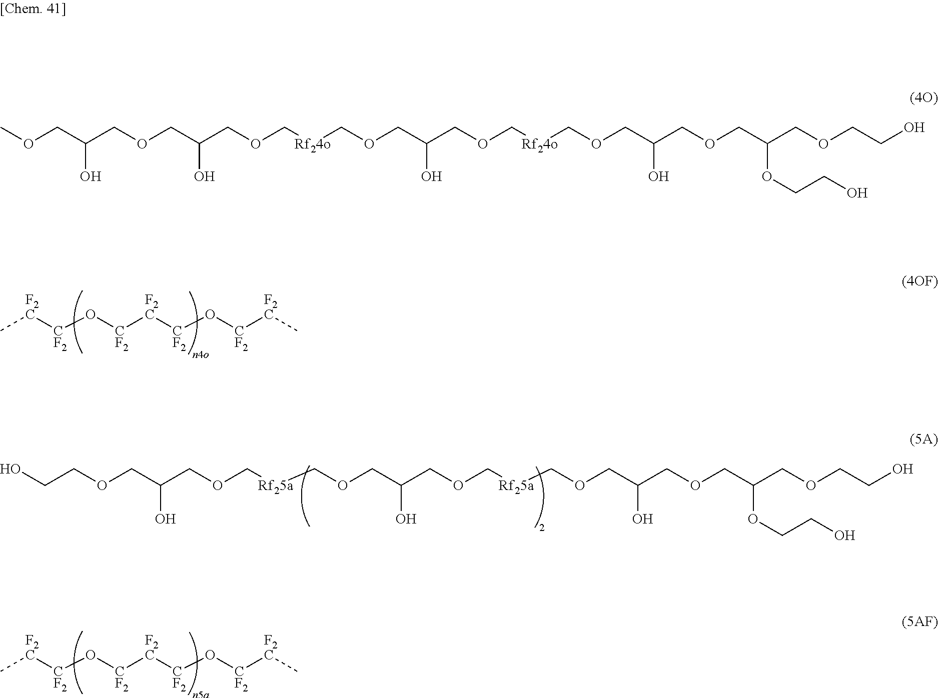

(in Formula (40), Rf₂40 is represented by Formula (4° F.), in Rf₂40, n40 indicates an average degree of polymerization and represents 0.1 to 15, and in Formula (40), in two Rf₂40's, n40's may be the same as or different from each other).
(in Formula (5A), Rf₂5a is represented by Formula (5AF), in Rf₂5a, n5a indicates an average degree of polymerization and represents 0.1 to 15, and in Formula (5A), in three Rf₂5a's, n5a's may be different from each other, or some or all of them may be the same).

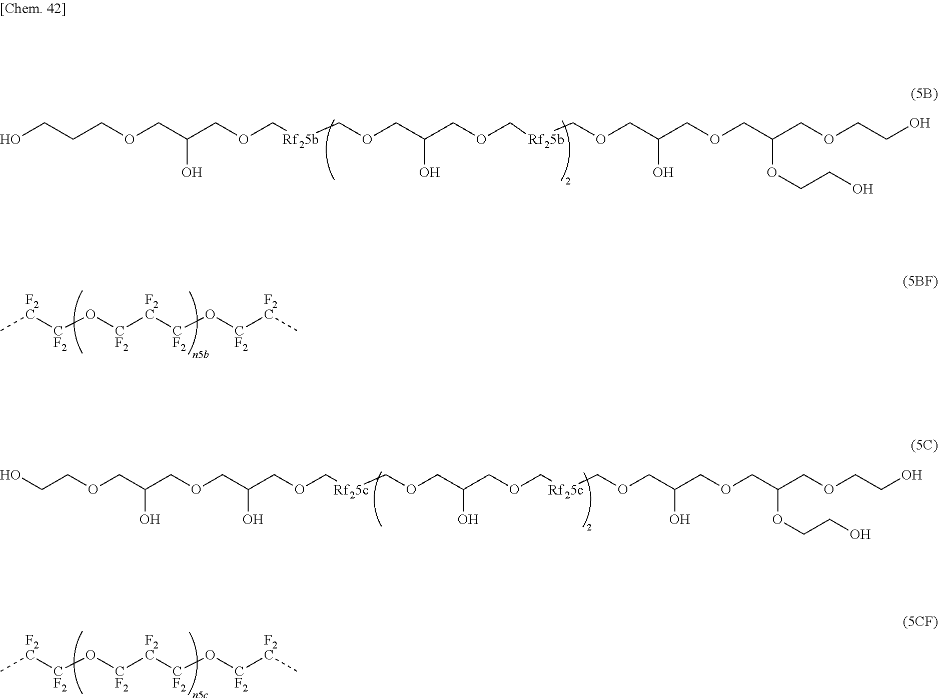

(in Formula (5B), Rf₂5b is represented by Formula (5BF), in Rf₂5b, n5b indicates an average degree of polymerization and represents 0.1 to 15, and in Formula (5B), in three Rf₂5b's, n5b's may be different from each other, or some or all of them may be the same).
(in Formula (5C), Rf₂5c is represented by Formula (5CF), in Rf₂5c, nSc indicates an average degree of polymerization and represents 0.1 to 15, and in Formula (5C), in three Rf₂5c's, n5c's may be different from each other, or some or all of them may be the same).

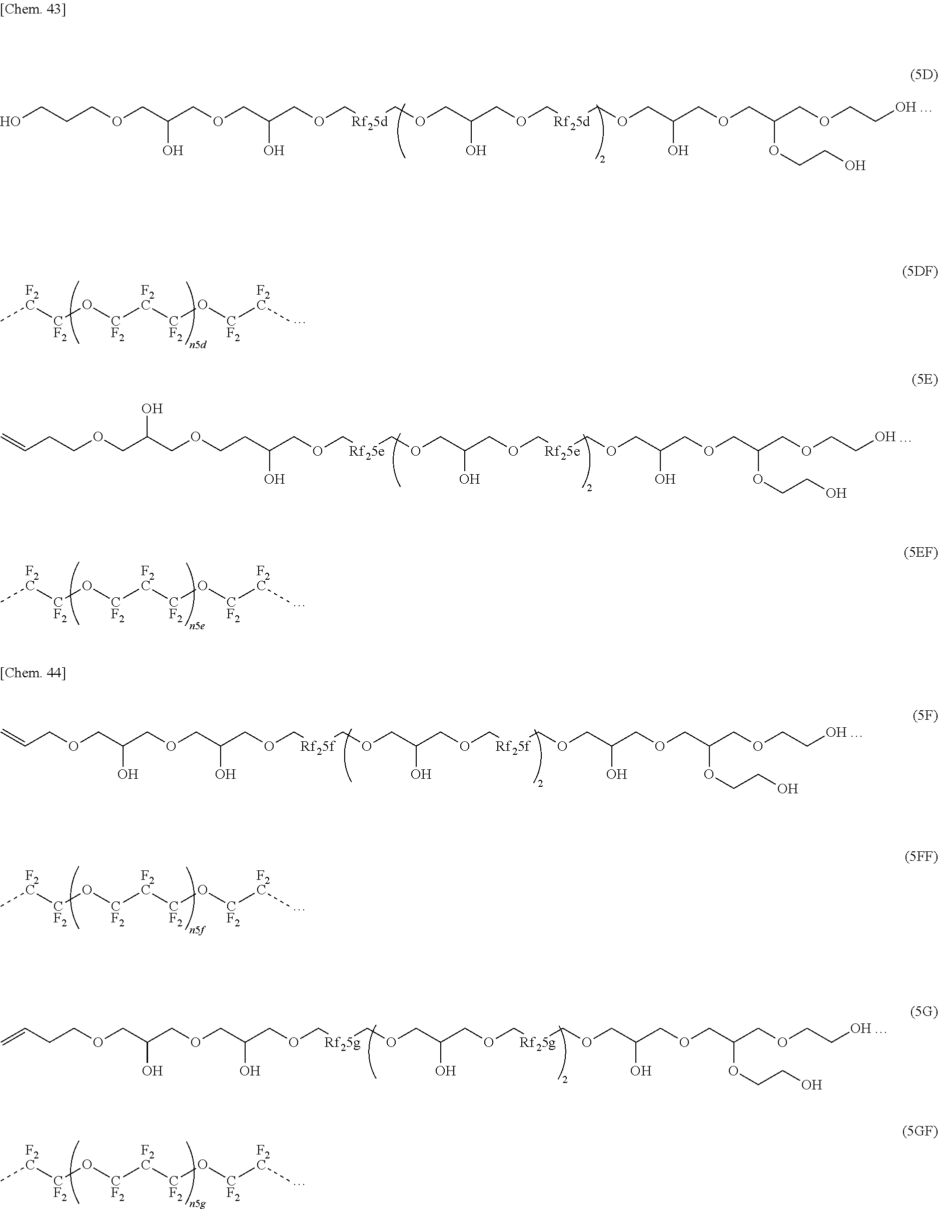

(in Formula (5D), Rf₂5d is represented by Formula (5DF), in Rf₂5d, n5d indicates an average degree of polymerization and represents 0.1 to 15, and in Formula (5D), in three Rf₂5d's, n5d's may be different from each other, or some or all of them may be the same).
(in Formula (5E), Rf₂5e is represented by Formula (5EF), in Rf₂5e, n5e indicates an average degree of polymerization and represents 0.1 to 15, and in Formula (5E), in three Rf₂5e's, n5e's may be different from each other, or some or all of them may be the same).
(in Formula (5F), Rf₂5f is represented by Formula (5FF), in Rf₂5f, n5f indicates an average degree of polymerization and represents 0.1 to 15, and in Formula (5F), in three Rf₂5f's, n5f's may be different from each other, or some or all of them may be the same).
(in Formula (5G), Rf₂5g is represented by Formula (5GF), in Rf₂5g, n5g indicates an average degree of polymerization and represents 0.1 to 15, and in Formula (5G), in three Rf₂5g's, n5g's may be different from each other, or some or all of them may be the same).

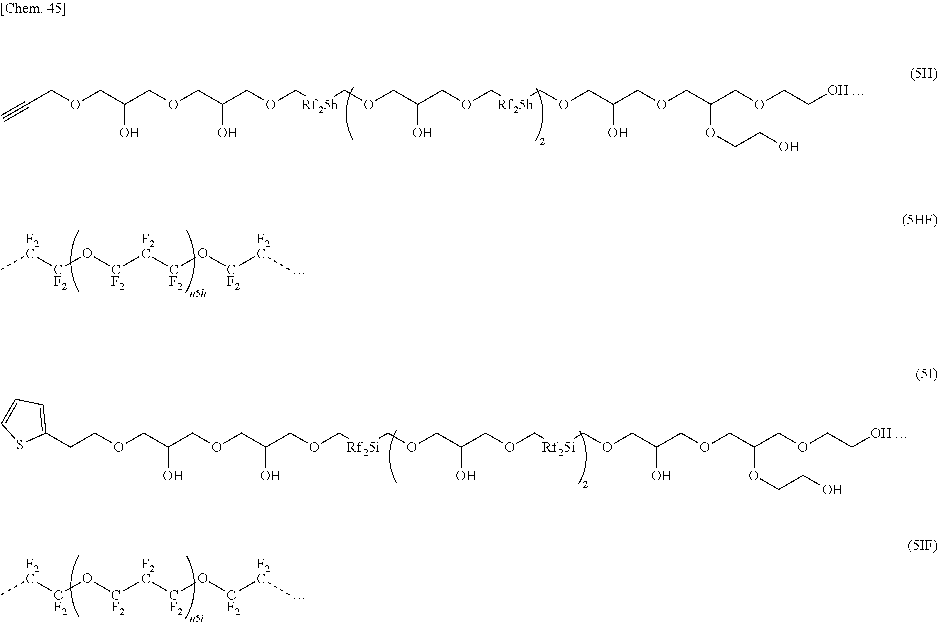

(in Formula (5H), Rf₂5h is represented by Formula (5HF), in Rf₂5h, n5h indicates an average degree of polymerization and represents 0.1 to 15, and in Formula (5H), in three Rf₂5h's, n5h's may be different from each other, or some or all of them may be the same).
(in Formula (51), Rf₂5i is represented by Formula (5IF), in Rf₂5i, n5i indicates an average degree of polymerization and represents 0.1 to 15, and in Formula (51), in three Rf₂5i's, n5i's may be different from each other, or some or all of them may be the same).

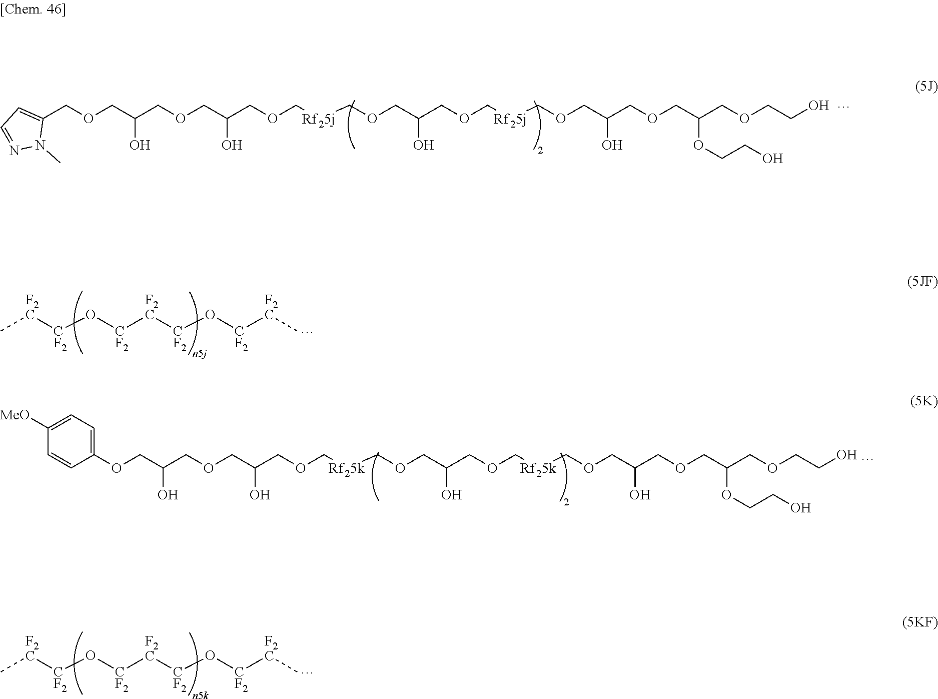

(in Formula (5J), Rf₂5j is represented by Formula (5JF), in Rf₂5j, n5j indicates an average degree of polymerization and represents 0.1 to 15, and in Formula (5J), in three Rf₂5j's, n5j's may be different from each other, or some or all of them may be the same).
(in Formula (5K), Rf₂5k is represented by Formula (5KF), Me represents a methyl group, in Rf₂5k, n5k indicates an average degree of polymerization and represents 0.1 to 15, and in Formula (5K), in three Rf₂5k's, n5k's may be different from each other, or some or all of them may be the same).

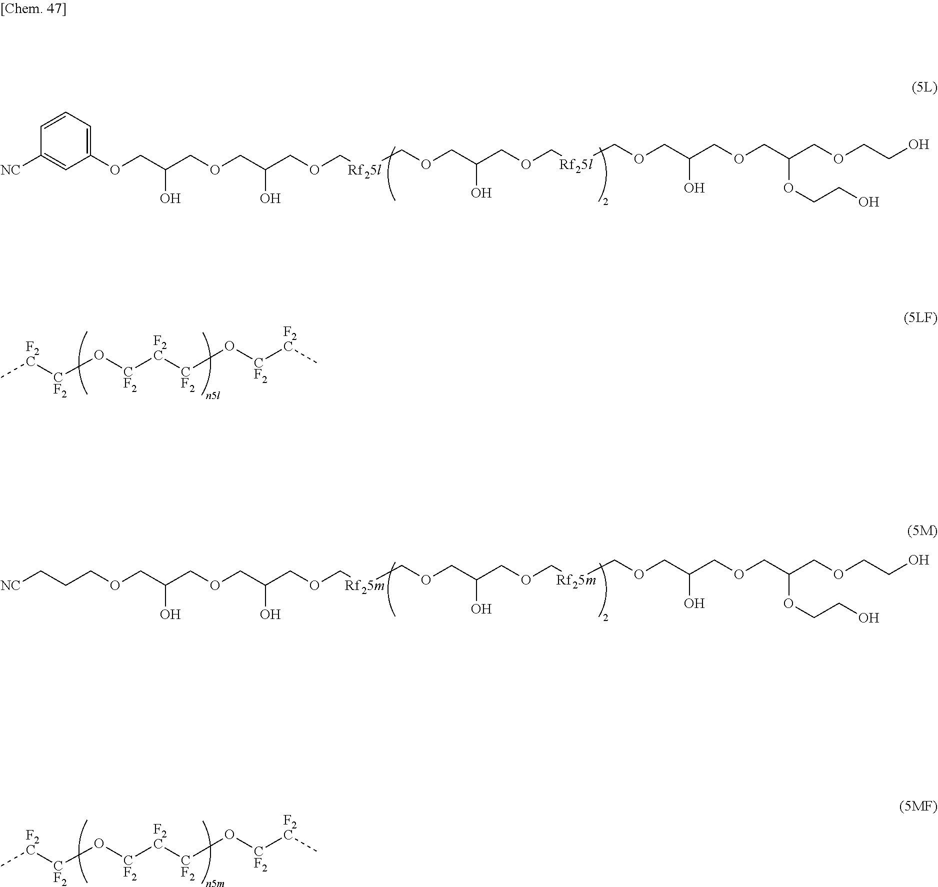

(in Formula (5L), Rf₂51 is represented by Formula (5LF), and in Rf₂51, n51 indicates an average degree of polymerization and represents 0.1 to 15, and in Formula (5L), in three Rf₂5l's, n5l's may be different from each other, or some or all of them may be the same).
(in Formula (5M), Rf₂5m is represented by Formula (5MF), and in Rf₂5m, n5m indicates an average degree of polymerization and represents 0.1 to 15, and in Formula (5M), in three Rf₂5m's, n5m's may be different from each other, or some or all of them may be the same).

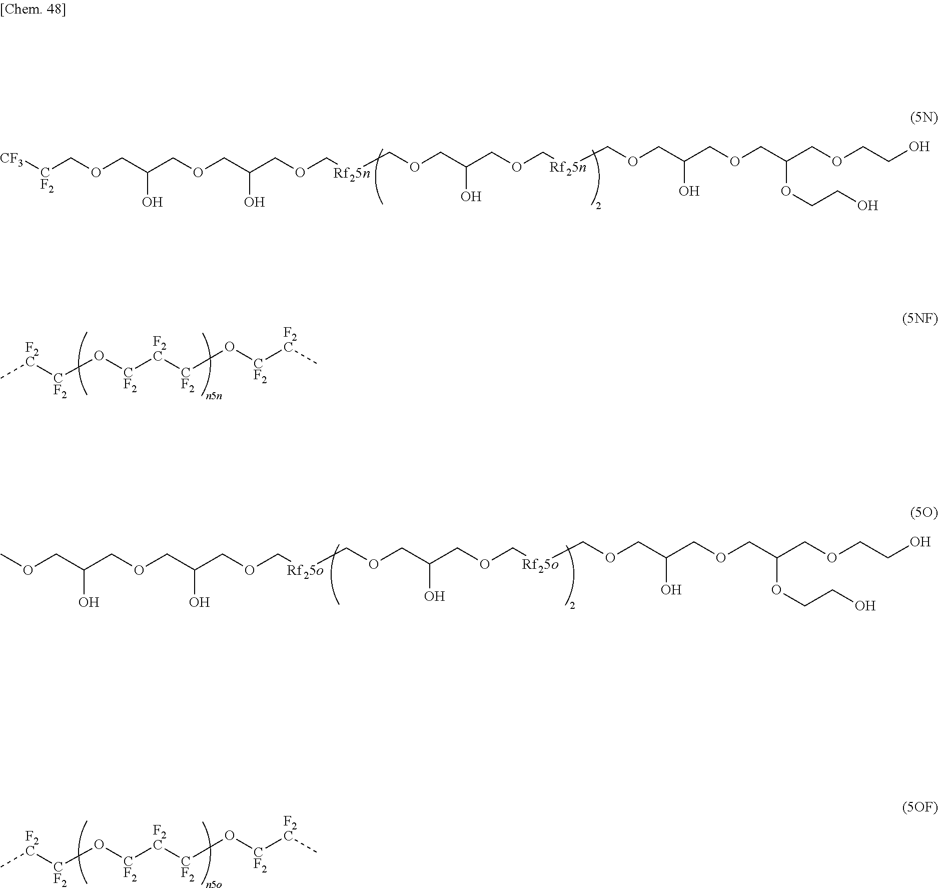

(in Formula (5N), Rf₂5n is represented by Formula (5NF), and in Rf₂5n, n5n indicates an average degree of polymerization and represents 0.1 to 15, and in Formula (5N), in three Rf₂5n's, non's may be different from each other, or some or all of them may be the same).
(in Formula (5O), Rf₂5o is represented by Formula (5OF), and in Rf₂5o, n5o indicates an average degree of polymerization and represents 0.1 to 15, and in Formula (50), in three Rf₂5o's, n5o's may be different from each other, or some or all of them may be the same).

The values of z when Formulae (4A) to (40), and (5A) to (50) were applied to Formula (1), and the structures of R¹, [A], [B], R², R³, [C], [D], and R⁴ are shown in Table 1 to Table 4.

TABLE 1

| | | | | | | R³ = | | | |
|---|---|---|---|---|---|---|---|---|---|
| Compound | z | R¹ | [B] | [A] | R² | (Formula (5)) | [C] | [D] | R⁴ |
| (4A) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 2, v = 1 | b = 0 | a = 1 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (4B) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 3, v = 1 | b = 0 | a = 1 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (4C) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 2, v = 1 | b = 0 | a = 1 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (4D) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 3, v = 1 | b = 0 | a = 1 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (4E) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | v = 0 | b = 0 | a = 2 | | | d = 1 | e = 0 | g = 1 |
| | | R⁵ = 3- | | | | | | | X¹ = X² = |
| | | butenyl | | | | | | | Formula (7) |
| | | group | | | | | | | s = 2, t = 1 |
| (4F) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | v = 0 | b = 0 | a = 2 | | | d = 1 | e = 0 | g = 1 |
| | | R⁵ = allyl | | | | | | | X¹ = X² = |
| | | group | | | | | | | Formula (7) |
| | | | | | | | | | s = 2, t = 1 |
| (4G) | 1 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | v = 0 | b = 0 | a = 2 | | | d = 1 | e = 0 | g = 1 |
| | | R⁵ = 3- | | | | | | | X¹ = X² = |
| | | butenyl | | | | | | | Formula (7) |
| | | group | | | | | | | s = 2, t = 1 |

TABLE 2

| | | | | | | R³ = | | | |
|---|---|---|---|---|---|---|---|---|---|
| Compound | z | R¹ | [B] | [A] | R² | (Formula (5)) | [C] | [D] | R⁴ |
| (4H) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = propargyl | b = 0 | a = 2 | | | d = 1 | e = 0 | g = 1 |
| | | group | | | | | | | X¹ = X² = |
| | | | | | | | | | Formula (7) |
| | | | | | | | | | s = 2, t = 1 |
| (4I) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = thienylethyl | b = 0 | a = 2 | | | d = 1 | e = 0 | g = 1 |
| | | group | | | | | | | X¹ = X² = |
| | | | | | | | | | Formula (7) |
| | | | | | | | | | s = 2, t = 1 |
| (4J) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = N- | b = 0 | a = 2 | | | d = 1 | e = 0 | g = 1 |
| | | methylpyrazol | | | | | | | X¹ = X² = |
| | | group | | | | | | | Formula (7) |
| | | | | | | | | | s = 2, t = 1 |
| (4K) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = | b = 0 | a = 2 | | | d = 1 | e = 0 | g = 1 |
| | | methoxyphenyl | | | | | | | X¹ = X² = |
| | | group | | | | | | | Formula (7) |
| | | | | | | | | | s = 2, t = 1 |
| (4L) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = | b = 0 | a = 2 | | | d = 1 | e = 0 | g = 1 |
| | | cyanophenyl | | | | | | | X¹ = X² = |
| | | group | | | | | | | Formula (7) |
| | | | | | | | | | s = 2, t = 1 |
| (4M) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = 3- | b = 0 | a = 2 | | | d = 1 | e = 0 | g = 1 |
| | | cyanopropyl | | | | | | | X¹ = X² = |
| | | group | | | | | | | Formula (7) |
| | | | | | | | | | s = 2, t = 1 |
| (4N) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = 2,2,3,3,3- | b = 0 | a = 2 | | | d = 1 | e = 0 | g = 1 |
| | | pentafluoropropyl | | | | | | | X¹ = X² = |
| | | group | | | | | | | Formula (7) |
| | | | | | | | | | s = 2, t = 1 |
| (4O) | 1 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = methyl | b = 0 | a = 2 | | | d = 1 | e = 0 | g = 1 |
| | | group | | | | | | | X¹ = X² = |
| | | | | | | | | | Formula (7) |
| | | | | | | | | | s = 2, t = 1 |

TABLE 3

| | | | | | | R³ = | | | |
|---|---|---|---|---|---|---|---|---|---|
| Compound | z | R¹ | [B] | [A] | R² | (Formula (5)) | [C] | [D] | R⁴ |
| (5A) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 2, v = 1 | b = 0 | a = 1 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (5B) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 3, v = 1 | b = 0 | a = 1 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (5C) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 2, v = 1 | b = 0 | a = 2 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (5D) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | u = 3, v = 1 | b = 0 | a = 2 | | | d = 1 | e = 0 | g = 1 |
| | | R⁵ = hydrogen | | | | | | | X¹ = X² = |
| | | atom | | | | | | | Formula (7) |
| | | | | | | | | | s = 2, t = 1 |
| (5E) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | v = 0 | b = 1 | a = 1 | | | d = 1 | e = 0 | g = 1 |
| | | R⁵ = 3-butenyl | c = 2 | | | | | | X¹ = X² = |
| | | group | | | | | | | Formula (7) |
| | | | | | | | | | s = 2, t = 1 |
| (5F) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | v = 0 | b = 0 | a = 2 | | | d = 1 | e = 0 | g = 1 |
| | | R⁵ = allyl | | | | | | | X¹ = X² = |
| | | group | | | | | | | Formula (7) |
| | | | | | | | | | s = 2, t = 1 |
| (5G) | 2 | Formula | Formula | Formula | Formula | y1 = 1 | Formula | Formula | Formula |
| | | (8) | (2-2) | (2-1) | (10-2) | y2 = 1 | (3-1) | (3-2) | (6-1) |
| | | v = 0 | b = 0 | a = 2 | | | d = 1 | e = 0 | g = 1 |
| | | R⁵ = 3-butenyl | | | | | | | X¹ = X² = |
| | | atom | | | | | | | Formula (7) |
| | | | | | | | | | s = 2, t = 1 |

TABLE 4

| | | | | | | R³ = | | | |
|---|---|---|---|---|---|---|---|---|---|
| Compound | z | R¹ | [B] | [A] | R² | (Formula (5)) | [C] | [D] | R⁴ |
| (5H) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = propargyl | b = 0 | a = 2 | | | d = 1 | e = 0 | g = 1 |
| | | group | | | | | | | X¹ = X² = |
| | | | | | | | | | Formula (7) |
| | | | | | | | | | s = 2, t = 1 |
| (5I) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = | b = 0 | a = 2 | | | d = 1 | e = 0 | g = 1 |
| | | thienylethyl | | | | | | | X¹ = X² = |
| | | group | | | | | | | Formula (7) |
| | | | | | | | | | s = 2, t = 1 |
| (5J) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = N- | b = 0 | a = 2 | | | d = 1 | e = 0 | g = 1 |
| | | methylpyrazolyl- | | | | | | | X¹ = X² = |
| | | methyl | | | | | | | Formula (7) |
| | | group | | | | | | | s = 2, t = 1 |
| (5K) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = methoxy- | b = 0 | a = 2 | | | d = 1 | e = 0 | g = 1 |
| | | phenyl group | | | | | | | X¹ = X² = |
| | | | | | | | | | Formula (7) |
| | | | | | | | | | s = 2, t = 1 |
| (5L) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = cyano- | b = 0 | a = 2 | | | d = 1 | e = 0 | g = 1 |
| | | phenyl group | | | | | | | X¹ = X² = |
| | | | | | | | | | Formula (7) |
| | | | | | | | | | s = 2, t = 1 |
| (5M) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = 3- | b = 0 | a = 2 | | | d = 1 | e = 0 | g = 1 |
| | | cyanopropyl | | | | | | | X¹ = X² = |
| | | group | | | | | | | Formula (7) |
| | | | | | | | | | s = 2, t = 1 |
| (5N) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = 2,2,3,3,3- | b = 0 | a = 2 | | | d = 1 | e = 0 | g = 1 |
| | | pentafluoro- | | | | | | | X¹ = X² = |
| | | propyl group | | | | | | | Formula (7) |
| | | | | | | | | | s = 2, t = 1 |
| (5O) | 2 | Formula (8) | Formula | Formula | Formula | y1 = 1 | Formula | Formula | Formula |
| | | v = 0 | (2-2) | (2-1) | (10-2) | y2 = 1 | (3-1) | (3-2) | (6-1) |
| | | R⁵ = methyl | b = 0 | a = 2 | | | d = 1 | e = 0 | g = 1 |
| | | group | | | | | | | X¹ = X² = |
| | | | | | | | | | Formula (7) |
| | | | | | | | | | s = 2, t = 1 |

When the fluorine-containing ether compound represented by Formula (1) is a compound represented by any of Formulae (1A) to (IV), (2A) to (2V), (4A) to (40), and (5A) to (50), this is preferable because it is possible to form a lubricating layer which has a strong effect of inhibiting corrosion of the magnetic recording medium and can minimize the occurrence of pickup and spin-off even if the thickness is thin.

The number-average molecular weight (Mn) of the fluorine-containing ether compound of the present embodiment is preferably in a range of 500 to 10,000 and particularly preferably in a range of 600 to 5,000. When the number-average molecular weight is 500 or more, the lubricating layer composed of the lubricant containing the fluorine-containing ether compound of the present embodiment has excellent heat resistance. The number-average molecular weight of the fluorine-containing ether compound is more preferably 600 or more. In addition, when the number-average molecular weight is 10,000 or less, the viscosity of the fluorine-containing ether compound becomes appropriate, and when a lubricant containing this is applied, a lubricating layer having a thin film thickness can be easily formed. The number-average molecular weight of the fluorine-containing ether compound is more preferably 5,000 or less because the viscosity becomes one that makes the lubricant easy to handle.

The number-average molecular weight (Mn) of the fluorine-containing ether compound is a value measured through ¹H-NMR and ¹⁹F-NMR using a AVANCE III 400 (commercially available from Bruker BioSpin). Specifically, the number of repeating units of the PFPE chain is calculated from the integrated value measured by ¹⁹F-NMR to obtain a number-average molecular weight. In the measurement of nuclear magnetic resonance (NMR), a sample is diluted with a hexafluorobenzene/d-acetone (4/1v/v) solvent and used for measurement. The reference for ¹⁹F-NMR chemical shift is-164.7 ppm for the peak of hexafluorobenzene, and the reference for ¹H-NMR chemical shift is 2.2 ppm for the peak of acetone.

The fluorine-containing ether compound of the present embodiment preferably has a molecular weight dispersity (a ratio of the weight-average molecular weight (Mw)/the number-average molecular weight (Mn)) of 1.3 or less by molecular weight fractionation by an appropriate method.

In the present embodiment, the method for molecular weight fractionation is not particularly limited, and for example, molecular weight fractionation using a silica gel column chromatography method, a gel permeation chromatography (GPC) method or the like, molecular weight fractionation using a supercritical extraction method or the like can be used.

Here, the reason why it is possible to form a lubricating layer which has an effect of inhibiting corrosion of the magnetic recording medium and can minimize the occurrence of pickup and spin-off even if the thickness is thin when the lubricating layer is formed on the protective layer of the magnetic recording medium using the lubricant containing the fluorine-containing ether compound of the present embodiment.

In the fluorine-containing ether compound represented by Formula (1) according to the present embodiment, [A], [B], [C] and [D] are each a divalent linking group having a secondary hydroxy group. Here, in the fluorine-containing ether compound of the present embodiment, a —[B]-[A]- structure including 1 to 3 secondary hydroxy groups (hereinafter sometimes abbreviated as a “BA structure”) and a —[C]-[D]- structure including 1 to 2 secondary hydroxy groups (hereinafter sometimes abbreviated as a “CD structure”) are arranged in a well-balanced manner at both ends of —R²[—CH₂—R³—CH₂—R²]_{z}— via a methylene group (—CH₂—). Furthermore, etheric oxygen atoms of the BA structure and the CD structure impart appropriate flexibility to the molecular structure of the fluorine-containing ether compound represented by Formula (1).

In addition, when the BA structure and/or the CD structure includes a plurality of secondary hydroxy groups, carbon atoms to which the secondary hydroxy groups are bonded are bonded via a linking group composed of a methylene group (—CH₂—) and an ether bond (—O—). Therefore, even when the BA structure and/or the CD structure include a plurality of secondary hydroxy groups, the distance between adjacent secondary hydroxy groups is appropriate, and the secondary hydroxy groups are arranged to be easily adsorbed to the protective layer.

Accordingly, when a lubricating layer containing the fluorine-containing ether compound of the present embodiment is formed on the protective layer, the secondary hydroxy groups contained in the BA structure and the secondary hydroxy groups contained in the CD structure effectively participate in bonding with the active sites on the protective layer.

In addition, in Formula (1), R⁴ is a branched terminal group containing two or three primary hydroxy groups. Since the primary hydroxy group has less steric hindrance than the secondary hydroxy group and the tertiary hydroxy group, it effectively participates in the formation of intermolecular hydrogen bonds within the fluorine-containing ether compound.

In this manner, in the fluorine-containing ether compound represented by Formula (1) according to the present embodiment, <1> the secondary hydroxy group contained in the BA structure and the secondary hydroxy group contained in the CD structure effectively participate in bonding with the active sites on the protective layer, and <2> the plurality of primary hydroxy groups contained in R⁴ participate in the formation of intermolecular hydrogen bonds within the fluorine-containing ether compound.

Therefore, in the lubricating layer containing the fluorine-containing ether compound of the present embodiment, an excellent adsorption force for the protective layer exhibited by the secondary hydroxy groups arranged at both ends of —R²[—CH₂—R³—CH₂—R²]_{z}— in Formula (1) and an excellent intermolecular force exhibited by the primary hydroxy group of the branched terminal group act effectively in a well-balanced manner.

In addition, in the lubricating layer containing the fluorine-containing ether compound of the present embodiment, <3> sufficient hydrophobicity is obtained by inclusion of two or three PFPE chains represented by R² in Formula (1), and moreover, R³ having a secondary hydroxy group arranged between adjacent R²'s prevents R² from moving too far away from the protective layer.

Accordingly, the lubricating layer containing the fluorine-containing ether compound of the present embodiment has the functions <1> to <3>, and the synergistic effect of these functions provides sufficient adhesion to the protective layer and an effect of inhibiting corrosion of a magnetic recording medium. As a result, in the magnetic recording medium having a lubricating layer containing the fluorine-containing ether compound of the present embodiment, it is possible to prevent the fluorine-containing ether compound that is present without adhering (adsorbing) to the protective layer from adhering to a magnetic head as foreign matter (smear), and minimize the occurrence of pickup. In addition, the occurrence of spin-off, in which the film thickness of the lubricating layer is reduced by scattering and evaporating the lubricant due to a centrifugal force and/or heat generated according to rotation of the magnetic recording medium at a high speed, is minimized. In addition, the magnetic recording medium has excellent corrosion resistance and favorable reliability and durability.

### “Production Method”

The method of producing the fluorine-containing ether compound of the present embodiment is not particularly limited, and conventionally known production methods can be used for production. The fluorine-containing ether compound of the present embodiment can be produced using, for example, the following production methods.

[First Production Method (when z is 1)]
(When R¹—[B]-[A]-and-[C]-[D]-R⁴ are the same, and two R²'s are the Same)

First, a fluorine-based compound in which hydroxymethyl groups (—CH₂OH) are arranged at both terminals of a PFPE chain corresponding to R² in Formula (1) is prepared. Next, a hydroxy group of a hydroxymethyl group arranged at one terminal of the fluorine-based compound is reacted with an epoxy compound corresponding to a group composed of R¹—[B]-[A]-(=—[C]-[D]-R⁴) in Formula (1) (first reaction). Accordingly, an intermediate compound 1-1 having a group corresponding to R¹—[B]-[A]-(=—[C]-[D]-R⁴) at one terminal of the PFPE chain corresponding to R² is obtained.

In the first reaction, the hydroxy group of the epoxy compound corresponding to a group composed of R¹—[B]-[A]-(=—[C]-[D]-R⁴) may be protected using an appropriate protecting group and then reacted with the fluorine-based compound.

The epoxy compound corresponding to a group composed of R¹—[B]-[A]- (or —[C]-[D]-R⁴) used when the fluorine-containing ether compound of the present embodiment is produced may be synthesized, and a commercial product may be purchased and used.

When the epoxy compound is synthesized, for example, a method of reacting an alcohol having a structure corresponding to a group composed of R¹—[B]-[A]- (or —[C]-[D]-R⁴) of a fluorine-containing ether compound to be produced with a compound having an epoxy group can be used. In this method, as the compound having an epoxy group, for example, any compound selected from among epichlorohydrin, epibromohydrin, 2-bromoethyloxirane, and allyl glycidyl ether can be used. In addition, as another method of synthesizing the epoxy compound, a method of preparing an unsaturated compound having a structure corresponding to a group composed of R¹—[B]-[A]- (or —[C]-[D]-R⁴) of a fluorine-containing ether compound to be produced and oxidizing the unsaturated bond may be used.

Next, a hydroxy group of a hydroxymethyl group arranged at a terminal of the intermediate compound 1-1 is reacted with a halogen compound having an epoxy group corresponding to R³. Then, the obtained epoxy compound is additionally reacted with a hydroxy group arranged at a terminal of one more molecule of the intermediate compound 1-1 (second reaction).

As the halogen compound having an epoxy group corresponding to R³, for example, when R³ is represented by Formula (5), and in Formula (5), y1 and y2 are both 1, epibromohydrin can be used.

When the above step is performed, a compound in which, Formula (1), z is 1, R¹—[B]-[A]-and-[C]-[D]-R⁴ are the same, and two PFPE chains represented by R² are the same can be produced.

(When R¹—[B]-[A]-and-[C]-[D]-R⁴ are different and/or two R²'s are Different)

In the first reaction in the first production method, in the same manner as in the case of producing a compound in which R¹—[B]-[A]-and-[C]-[D]-R⁴ are the same, and two PFPE chains represented by R² are the same, an intermediate compound 1-2 having a group corresponding to R¹—[B]-[A]- at one terminal of the PFPE chain corresponding to R² on the side of R¹ and a hydroxymethyl group arranged at the other terminal is produced.

In addition, when R¹—[B]-[A]-and-[C]-[D]-R⁴ are different, and/or two PFPE chains represented by R² are different, in the first reaction, in the same manner as the intermediate compound 1-2 having a group corresponding to R¹—[B]-[A]- at one terminal, an intermediate compound 1-3 having a group corresponding to —[C]-[D]-R⁴ at one terminal of the PFPE chain corresponding to R² on the side of R⁴ and a hydroxymethyl group arranged at the other terminal is produced.

Then, an epoxy compound is obtained by reacting the intermediate compound 1-2 with a halogen compound having an epoxy group corresponding to R³. Subsequently, the obtained epoxy compound is reacted with the intermediate compound 1-3 (second reaction).

Here, in the second reaction, an example in which an epoxy compound obtained by reacting the intermediate compound 1-2 with a halogen compound having an epoxy group corresponding to R³ is reacted with the intermediate compound 1-3 has been exemplified, but an epoxy compound obtained by reacting the intermediate compound 1-3 with a halogen compound having an epoxy group corresponding to R³ may be reacted with the intermediate compound 1-2.

When the above step is performed, a compound in which, in Formula (1), z is 1, R¹—[B]-[A]-and-[C]-[D]-R⁴ are different, and/or two PFPE chains represented by R² are different can be produced.

[Second Production Method (when z is 2)] (when R¹—[B]-[A]-and-[C]-[D]-R⁴ are the same, two R³'s are the same, and three R²'s are the Same)

First, a fluorine-based compound in which hydroxymethyl groups (—CH₂OH) are arranged at both terminals of a PFPE chain corresponding to R² in Formula (1) is prepared. Next, hydroxy groups of hydroxymethyl groups arranged at both terminals of the fluorine-based compound are reacted with a halogen compound having an epoxy group corresponding to R³ (first reaction). Accordingly, an intermediate compound 2-1 having an epoxy group corresponding to R³ at both terminals of a PFPE chain corresponding to R² is obtained.

In addition, a fluorine-based compound in which hydroxymethyl groups (—CH₂OH) are arranged at both terminals of a PFPE chain corresponding to R² in Formula (1) is prepared. Then, a hydroxy group of a hydroxymethyl group arranged at one terminal of the fluorine-based compound is reacted with an epoxy compound corresponding to a group composed of R¹—[B]-[A]-(=—[C]-[D]-R⁴) in Formula (1) (second reaction). Accordingly, an intermediate compound 2-2 having a group corresponding to R¹—[B]-[A]-(=—[C]-[D]-R⁴) at one terminal of a PFPE chain corresponding to R² and a hydroxymethyl group arranged at the other terminal is obtained.

In the second reaction, the hydroxy group of the epoxy compound corresponding to a group composed of R¹—[B]-[A]-(=—[C]-[D]-R⁴) may be protected using an appropriate protecting group and then reacted with the fluorine-based compound.

In addition, the second reaction in the second production method may be performed after the first reaction or before the first reaction.

Then, a hydroxy group of a hydroxymethyl group arranged at one terminal of the intermediate compound 2-2 is reacted with epoxy groups arranged at both terminals of the intermediate compound 2-1 (third reaction).

When the above step is performed, it is possible to produce a compound in which, in Formula (1), z is 2, two linking groups represented by R³ are the same, R¹—[B]-[A]-and-[C]-[D]-R⁴ are the same, and three PFPE chains represented by R² are the same.

(When Two R³'s are the same, R¹—[B]-[A]-and-[C]-[D]-R⁴ are different, and/or any one or more of three R²'s are Different)

In the first reaction in the second production method, an intermediate compound 2-1 having an epoxy group corresponding to R³ at both terminals of a PFPE chain corresponding to R² in the center of the molecule is synthesized.

Next, in the second reaction, in the same manner as the intermediate compound 2-2, the following intermediate compound 2-2a and intermediate compound 2-2b are synthesized.

Specifically, a fluorine-based compound in which hydroxymethyl groups are arranged at both terminals of a PFPE chain corresponding to R² on the side of R¹ is prepared. Then, a hydroxy group of a hydroxymethyl group arranged at one terminal of the fluorine-based compound is reacted with an epoxy compound corresponding to a group composed of R¹—[B]-[A]-in Formula (1). Accordingly, an intermediate compound 2-2a in which one terminal of a PFPE chain corresponding to R² on the side of R¹ has a group corresponding to R¹—[B]-[A]- and a hydroxymethyl group is arranged at the other terminal is obtained.

In addition, a fluorine-based compound in which hydroxymethyl groups are arranged at both terminals of a PFPE chain corresponding to R² on the side of R⁴ is prepared. Then, a hydroxy group of a hydroxymethyl group arranged at one terminal of the fluorine-based compound is reacted with an epoxy compound corresponding to a group composed of —[C]-[D]-R⁴ in Formula (1). Accordingly, an intermediate compound 2-2b in which one terminal of a PFPE chain corresponding to R² on the side of R⁴ has a group corresponding to —[C]-[D]-R⁴ and a hydroxymethyl group is arranged at the other terminal is obtained.

Then, in the third reaction, the epoxy groups corresponding to R³ arranged at the terminals of the intermediate compound 2-1 are reacted with the intermediate compound 2-2a and the intermediate compound 2-2b. In the third reaction, the reaction between the terminal of the intermediate compound 2-1 and the intermediate compound 2-2a may be performed before and after the reaction between the terminal of the intermediate compound 2-1 and the intermediate compound 2-2b.

When the above step is performed, it is possible to produce a compound in which, in Formula (1), z is 2, two linking groups represented by R³ are the same, R¹—[B]-[A]-and-[C]-[D]-R⁴ are different, and/or any one or more of three PFPE chains represented by R² are different.

(When Two R³'s are different, R¹—[B]-[A]-and-[C]-[D]-R⁴ are the same, and three R²'s are the Same)

In the first reaction in the second production method, a halogen compound having an epoxy group corresponding to one R³ and a halogen compound having an epoxy group corresponding to the other R³ are reacted with hydroxy groups of hydroxymethyl groups arranged at the terminals of the fluorine-based compound by a known method. Accordingly, an intermediate compound 2-1b having an epoxy group corresponding to one R³ at one end of a PFPE chain corresponding to R² and an epoxy group corresponding to the other R³ at the other end is obtained.

Next, in the second reaction, in the same manner as when R¹—[B]-[A]-and-[C]-[D]-R⁴ are the same, two R³'s are the same, and three R²'s are the same, an intermediate compound 2-2 is synthesized.

Then, in the third reaction, the intermediate compound 2-1b and the intermediate compound 2-2 are reacted in the same manner as when R¹—[B]-[A]-and-[C]-[D]-R⁴ are the same, two R³'s are the same, and three R²'s are the same except that the intermediate compound 2-1b is used in place of the intermediate compound 2-1.

When the above step is performed, a compound in which, in Formula (1), z is 2, two linking groups represented by R³ are different, R¹—[B]-[A]-and-[C]-[D]-R⁴ are the same, and three PFPE chains represented by R² are the same can be produced.

(when Two R³'s are different, R¹—[B]-[A]-and-[C]-[D]-R⁴ are different, and/or any one or more of three R²'s are Different)

In the first reaction in the second production method, an intermediate compound 2-1b is synthesized in the same manner as when two R³'s are different, R¹—[B]-[A]-and-[C]-[D]-R⁴ are the same, and three R²'s are the same.

Then, in the second reaction, in the same manner as when two R³'s are the same, R¹—[B]-[A]-and-[C]-[D]-R⁴ are different, and/or any one or more of three R²'s are different, the intermediate compound 2-2a and the intermediate compound 2-2b are synthesized.

Then, in the third reaction, the epoxy group corresponding to R³ on the side of R¹ of the intermediate compound 2-1b is reacted with the intermediate compound 2-2a, and the epoxy group corresponding to R³ on the side of R⁴ of the intermediate compound 2-1b is reacted with the intermediate compound 2-2b in the same manner as when two R³'s are the same, R¹—[B]-[A]-and-[C]-[D]-R⁴ are different, and/or any one or more of three R²'s are different except that the intermediate compound 2-1b is used in place of the intermediate compound 2-1.

When the above step is performed, a compound in which, in Formula (1), z is 2, two linking groups represented by R³ are different, R¹—[B]-[A]-and-[C]-[D]-R⁴ are different, and/or any one or more of three PFPE chains represented by R² are different can be produced.

### “Lubricant for Magnetic Recording Medium”

The lubricant for magnetic recording medium of the present embodiment contains the fluorine-containing ether compound represented by Formula (1).

The lubricant of the present embodiment can be used by being mixed with a known material used as a material for the lubricant as necessary as long as the characteristics are not impaired due to the inclusion of the fluorine-containing ether compound represented by Formula (1).

Specific examples of known materials include, for example, FOMBLIN® ZDIAC, FOMBLIN ZDEAL, and FOMBLIN AM-2001 (all commercially available from Solvay Solexis), and Moresco A20H (commercially available from Moresco). A known material used in combination with the lubricant of the present embodiment preferably has a number-average molecular weight of 1,000 to 10,000.

When the lubricant of the present embodiment contains a material other than the fluorine-containing ether compound represented by Formula (1), the content of the fluorine-containing ether compound represented by Formula (1) in the lubricant of the present embodiment is preferably 50 mass % or more and more preferably 70 mass % or more.

Since the lubricant of the present embodiment contains the fluorine-containing ether compound represented by Formula (1), it is possible to form a lubricating layer having a strong effect of inhibiting corrosion of the magnetic recording medium, favorable adhesion to the protective layer, and favorable pickup characteristics and spin-off characteristics of the magnetic recording medium even if the thickness is thin.

### “Magnetic Recording Medium”

In a magnetic recording medium of the present embodiment, at least a magnetic layer, a protective layer, and a lubricating layer are sequentially provided on a substrate.

In the magnetic recording medium of the present embodiment, as necessary, one, two or more base layers can be provided between the substrate and the magnetic layer. In addition, at least one of the adhesive layer and the soft magnetic layer can be provided between the base layer and the substrate.

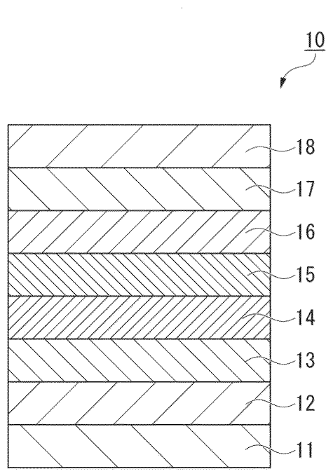

FIG. 1 is a schematic cross-sectional view showing a magnetic recording medium according to one embodiment of the present invention.

A magnetic recording medium 10 of the present embodiment has a structure in which an adhesive layer 12, a soft magnetic layer 13, a first base layer 14, a second base layer 15, a magnetic layer 16, a protective layer 17, and a lubricating layer 18 are sequentially provided on a substrate 11.

### “Substrate”

As the substrate 11, for example, a non-magnetic substrate in which a film made of NiP or a NiP alloy is formed on a base made of a metal or an alloy material such as Al or an Al alloy can be used.

In addition, as the substrate 11, a non-magnetic substrate made of a non-metallic material such as glass, a ceramic, silicon, silicon carbide, carbon, and a resin may be used, or a non-magnetic substrate in which a film of NiP or a NiP alloy is formed on a base made of these non-metallic materials may be used.

### “Adhesive Layer”

The adhesive layer 12 prevents the progress of corrosion of the substrate 11 that occurs when the substrate 11 and the soft magnetic layer 13 provided on the adhesive layer 12 are arranged in contact with each other.

The material of the adhesive layer 12 can be appropriately selected from among, for example, Cr, a Cr alloy, Ti, a Ti alloy, CrTi, NiAl, and an AlRu alloy. The adhesive layer 12 can be formed by, for example, a sputtering method.

### “Soft Magnetic Layer”

The soft magnetic layer 13 preferably has a structure in which a first soft magnetic film, an intermediate layer made of a Ru film, and a second soft magnetic film are sequentially laminated. That is, the soft magnetic layer 13 preferably has a structure in which an intermediate layer made of a Ru film is interposed between two soft magnetic film layers, and thus the soft magnetic films above and below the intermediate layer are bonded by anti-ferromagnetic coupling (AFC).

Examples of materials of the first soft magnetic film and the second soft magnetic film include a CoZrTa alloy and a CoFe alloy.

It is preferable to add any of Zr, Ta, and Nb to the CoFe alloy used for the first soft magnetic film and the second soft magnetic film. Thereby, the amorphization of the first soft magnetic film and the second soft magnetic film is promoted. As a result, the orientation of the first base layer (seed layer) can be improved, and the raised amount of the magnetic head can be reduced.

The soft magnetic layer 13 can be formed by, for example, a sputtering method.

### “First Base Layer”

The first base layer 14 is a layer that controls the orientation and the crystal size of the second base layer 15 and the magnetic layer 16 provided thereon.

Examples of the first base layer 14 include a Cr layer, a Ta layer, a Ru layer, a CrMo alloy layer, a CoW alloy layer, a CrW alloy layer, a CrV alloy layer, and a CrTi alloy layer.

The first base layer 14 can be formed by, for example, a sputtering method.

### “Second Base Layer”

The second base layer 15 is a layer that controls the orientation of the magnetic layer 16 such that it becomes favorable. The second base layer 15 is preferably a layer made of Ru or a Ru alloy.

The second base layer 15 may be a single layer or may be composed of a plurality of layers. When the second base layer 15 is composed of a plurality of layers, all of the layers may be composed of the same material, or at least one layer may be composed of a different material.

The second base layer 15 can be formed by, for example, a sputtering method.

### “Magnetic Layer”

The magnetic layer 16 is made of a magnetic film in which the axis of easy magnetization is in a direction perpendicular or horizontal to the surface of the substrate. The magnetic layer 16 is a layer containing Co and Pt. The magnetic layer 16 may be a layer containing an oxide, Cr, B, Cu, Ta, Zr or the like in order to improve SNR characteristics.

Examples of oxides contained in the magnetic layer 16 include SiO₂, SiO, Cr₂O₃, CoO, Ta₂O₃, and TiO₂.

The magnetic layer 16 may be composed of one layer or may be composed of a plurality of magnetic layers made of materials with different compositions.

For example, when the magnetic layer 16 is composed of three layers including a first magnetic layer, a second magnetic layer and a third magnetic layer sequentially laminated from below, the first magnetic layer preferably has a granular structure made of a material containing Co, Cr, and Pt, and further containing an oxide. As the oxide contained in the first magnetic layer, for example, it is preferable to use an oxide of Cr, Si, Ta, Al, Ti, Mg, Co or the like. Among these, particularly, TiO₂, Cr₂O₃, SiO₂ or the like can be preferably used. In addition, the first magnetic layer is preferably made of a composite oxide in which two or more oxides are added. Among these, particularly, Cr₂O₃—SiO₂, Cr₂O₃—TiO₂, SiO₂—TiO₂ or the like can be preferably used.

The first magnetic layer can contain one or more elements selected from among B, Ta, Mo, Cu, Nd, W, Nb, Sm, Tb, Ru, and Re in addition to Co, Cr, Pt, and an oxide.

For the second magnetic layer, the same material as for the first magnetic layer can be used. The second magnetic layer preferably has a granular structure.

The third magnetic layer preferably has a non-granular structure made of a material containing Co, Cr, and Pt and not containing an oxide. The third magnetic layer can contain one or more elements selected from among B, Ta, Mo, Cu, Nd, W, Nb, Sm, Tb, Ru, Re, and Mn in addition to Co, Cr, and Pt.

When the magnetic layer 16 is formed of a plurality of magnetic layers, it is preferable to provide a non-magnetic layer between adjacent magnetic layers. When the magnetic layer 16 is composed of three layers including a first magnetic layer, a second magnetic layer and a third magnetic layer, it is preferable to provide a non-magnetic layer between the first magnetic layer and the second magnetic layer and between the second magnetic layer and the third magnetic layer.

For the non-magnetic layer provided between adjacent magnetic layers of the magnetic layer 16, for example, Ru, a Ru alloy, a CoCr alloy, a CoCrX1 alloy (X1 represents one, two or more elements selected from among Pt, Ta, Zr, Re, Ru, Cu, Nb, Ni, Mn, Ge, Si, O, N, W, Mo, Ti, V, and B) or the like can be preferably used.

For the non-magnetic layer provided between adjacent magnetic layers of the magnetic layer 16, it is preferable to use an alloy material containing an oxide, a metal nitride, or a metal carbide. Specifically, as the oxide, for example, SiO₂, Al₂O₃, Ta₂O₅, Cr₂O₃, MgO, Y₂O₃, TiO₂ or the like can be used. As the metal nitride, for example, AlN, Si₃N₄, TaN, CrN or the like can be used. As the metal carbide, for example, TaC, BC, SiC or the like can be used.

The non-magnetic layer can be formed by, for example, a sputtering method.

The magnetic layer 16 is preferably a magnetic layer for perpendicular magnetic recording in which the axis of easy magnetization is in a direction perpendicular to the surface of the substrate in order to realize a higher recording density. The magnetic layer 16 may be a magnetic layer for in-plane magnetic recording.

The magnetic layer 16 may be formed by any conventionally known method such as a vapor deposition method, an ion beam sputtering method, and a magnetron sputtering method. The magnetic layer 16 is generally formed by a sputtering method.

### “Protective Layer”

The protective layer 17 protects the magnetic layer 16. The protective layer 17 may be composed of one layer or may be composed of a plurality of layers. As the protective layer 17, a carbon-based protective layer can be preferably used, and an amorphous carbon protective layer is particularly preferable. When the protective layer 17 is a carbon-based protective layer, this is preferable because the interaction with the polar group (particularly the hydroxy group) contained in the fluorine-containing ether compound in the lubricating layer 18 is further improved.

The adhesive force between the carbon-based protective layer and the lubricating layer 18 can be controlled by forming a carbon-based protective layer with hydrogenated carbon and/or nitrogenated carbon, and adjusting the hydrogen content and/or nitrogen content in the carbon-based protective layer. The hydrogen content in the carbon-based protective layer measured by a hydrogen forward scattering (HFS) is preferably 3 atom % to 20 atom %. In addition, the nitrogen content in the carbon-based protective layer measured through X-ray photoelectron spectroscopy (XPS) is preferably 4 atom % to 15 atom %.

Hydrogen and/or nitrogen contained in the carbon-based protective layer need not be uniformly contained through the entire carbon-based protective layer. For example, the carbon-based protective layer is preferably formed as a composition gradient layer in which nitrogen is contained in the protective layer 17 on the side of the lubricating layer 18 and hydrogen is contained in the protective layer 17 on the side of the magnetic layer 16. In this case, the adhesive force between the magnetic layer 16 and the lubricating layer 18, and the carbon-based protective layer is further improved.

The film thickness of the protective layer 17 is preferably 1 nm to 7 nm. When the film thickness of the protective layer 17 is 1 nm or more, the performance of the protective layer 17 can be sufficiently obtained. The film thickness of the protective layer 17 is preferably 7 nm or less in order to reduce the thickness of the protective layer 17.

As a film formation method for the protective layer 17, a sputtering method using a target material containing carbon, a chemical vapor deposition (CVD) method using a hydrocarbon raw material such as ethylene or toluene, an ion beam deposition (IBD) method or the like can be used.

When a carbon-based protective layer is formed as the protective layer 17, for example, a film can be formed by a DC magnetron sputtering method. Particularly, when a carbon-based protective layer is formed as the protective layer 17, it is preferable to form an amorphous carbon protective layer by a plasma CVD method. The amorphous carbon protective layer formed by the plasma CVD method has a uniform surface and low roughness.

### “Lubricating Layer”

The lubricating layer 18 prevents contamination of the magnetic recording medium 10. In addition, the lubricating layer 18 reduces a frictional force of a magnetic head of a magnetic recording and reproducing device, which slides on the magnetic recording medium 10, and improves the durability of the magnetic recording medium 10.

As shown in FIG. 1, the lubricating layer 18 is formed on and in contact with the protective layer 17. The lubricating layer 18 is formed by applying the lubricant for magnetic recording medium according to the embodiment described above to the protective layer 17. Therefore, the lubricating layer 18 contains the above fluorine-containing ether compound.

When the protective layer 17 arranged below the lubricating layer 18 is a carbon-based protective layer, particularly, the lubricating layer 18 is bonded to the protective layer 17 with a high bonding force. As a result, even if the thickness of the lubricating layer 18 is thin, it is easy to obtain the magnetic recording medium 10 in which the surface of the protective layer 17 is covered at a high coating rate, and it is possible to effectively prevent contamination of the surface of the magnetic recording medium 10.

The average film thickness of the lubricating layer 18 is preferably 0.5 nm (5 Å) to 2.0 nm (20 Å) and more preferably 0.5 nm (5 Å) to 1.2 nm (12 Å). When the average film thickness of the lubricating layer 18 is 0.5 nm or more, the lubricating layer 18 is formed with a uniform film thickness without forming an island shape or a mesh shape. Therefore, the surface of the protective layer 17 can be coated with the lubricating layer 18 at a high coating rate. In addition, when the average film thickness of the lubricating layer 18 is 2.0 nm or less, the lubricating layer 18 can be made sufficiently thin, and the raised amount of the magnetic head can be sufficiently reduced.

### “Method of Forming Lubricating Layer”

Examples of methods of forming the lubricating layer 18 include a method in which a magnetic recording medium during production in which respective layers up to the protective layer 17 are formed on the substrate 11 is prepared, and a lubricating layer forming solution is applied onto the protective layer 17 and dried.

The lubricating layer forming solution can be obtained by dispersing and dissolving the lubricant for magnetic recording medium according to the embodiment described above in a solvent as necessary, and adjusting the viscosity and concentration to be suitable for application methods.

Examples of solvents used for the lubricating layer forming solution include fluorine-based solvents such as Vertel® XF (product name, commercially available from Du Pont-Mitsui Fluorochemicals Co., Ltd.).

The method of applying a lubricating layer forming solution is not particularly limited, and examples thereof include a spin coating method, a spraying method, a paper coating method, and a dipping method.

When the dipping method is used, for example, the following method can be used. First, the substrate 11 in which respective layers up to the protective layer 17 are formed is immersed in the lubricating layer forming solution contained in an immersion tank of a dip coating device. Next, the substrate 11 is lifted from the immersion tank at a predetermined speed. Accordingly, the lubricating layer forming solution is applied to the surface of the protective layer 17 of the substrate 11.

When the dipping method is used, the lubricating layer forming solution can be uniformly applied to the surface of the protective layer 17, and the lubricating layer 18 with a uniform film thickness can be formed on the protective layer 17.

In the present embodiment, the substrate 11 in which the lubricating layer 18 is formed is preferably subjected to a heat treatment. When the heat treatment is performed, the adhesion between the lubricating layer 18 and the protective layer 17 is improved, and the adhesive force between the lubricating layer 18 and the protective layer 17 is improved.

The heat treatment temperature is preferably 100° C. to 180° C. and more preferably 100° C. to 160° C. When the heat treatment temperature is 100° C. or higher, an effect of improving the adhesion between the lubricating layer 18 and the protective layer 17 is sufficiently obtained. In addition, when the heat treatment temperature is 180° C. or lower, it is possible to prevent thermal decomposition of the lubricating layer 18 according to the heat treatment. The heat treatment time can be appropriately adjusted according to the heat treatment temperature, and is preferably 10 minutes to 120 minutes.

In the present embodiment, in order to further improve the adhesive force of the lubricating layer 18 with respect to the protective layer 17, an ultraviolet ray (UV) emitting treatment may be performed on the lubricating layer 18 before the heat treatment or after the heat treatment.

In the magnetic recording medium 10 of the present embodiment, at least the magnetic layer 16, the protective layer 17, and the lubricating layer 18 are sequentially provided on the substrate 11. In the magnetic recording medium 10 of the present embodiment, the lubricating layer 18 containing the above fluorine-containing ether compound is formed on and in contact with the protective layer 17. Even if the film thickness of the lubricating layer 18 is thin, a strong effect of inhibiting corrosion of the magnetic recording medium, and favorable pickup characteristics and spin-off characteristics are obtained. Accordingly, the magnetic recording medium 10 of the present embodiment has excellent reliability and durability. In addition, the magnetic recording medium 10 of the present embodiment can have a small raised amount of the magnetic head (for example, 10 nm or less), and operates stably for a long period of time even in a harsh environment due to diversity of applications. Therefore, the magnetic recording medium 10 of the present embodiment is particularly preferable as a magnetic disk mounted in a load unload (LUL) type magnetic disk device.

### EXAMPLES

Hereinafter, the present invention will be described in more detail with reference to examples and comparative examples. Here, the present invention is not limited only to the following examples.

### Example 1

The compound represented by Formula (1A) was produced by the following method.

### (First Reaction)

20 g of a compound (a number-average molecular weight of 909 and a molecular weight distribution of 1.1) represented by HOCH₂CF₂CF₂O(CF₂CF₂CF₂O)ₙCF₂CF₂CH₂OH (in the formula, n indicating an average degree of polymerization is 3.8), 5.3 g (a molecular weight of 404, 13.2 mmol) of a compound represented by the following Formula (12), and 20 mL of t-butanol were put into a 200 mL eggplant flask under a nitrogen gas atmosphere, and stirred at room temperature until they became uniform. 0.74 g of potassium tert-butoxide (a molecular weight of 112.21, 6.6 mmol) was additionally added to the uniform solution, and the mixture was stirred and reacted at 70° C. for 16 hours.

The reaction product obtained after the reaction was cooled to 25° C., transferred into a separatory funnel containing 100 mL of water, and extracted three times with 100 mL of ethyl acetate. The organic layer was washed with water and dehydrated with anhydrous sodium sulfate. After the desiccant was filtered, the filtrate was concentrated, and the residue was purified through silica gel column chromatography to obtain 11.6 g (a molecular weight of 1,313, 8.8 mmol) of a compound represented by the following Formula (13) as an intermediate compound 1-1.

The compound represented by the following Formula (12) was synthesized by the following method. 1 equivalent of 3-allyloxy-1,2-propanediol was reacted with 2 equivalents of 2-(2-bromoethoxy)tetrahydro-2H-pyran. The double bond of the obtained compound was oxidized with m-chloroperbenzoic acid to synthesize a compound represented by the following Formula (12).

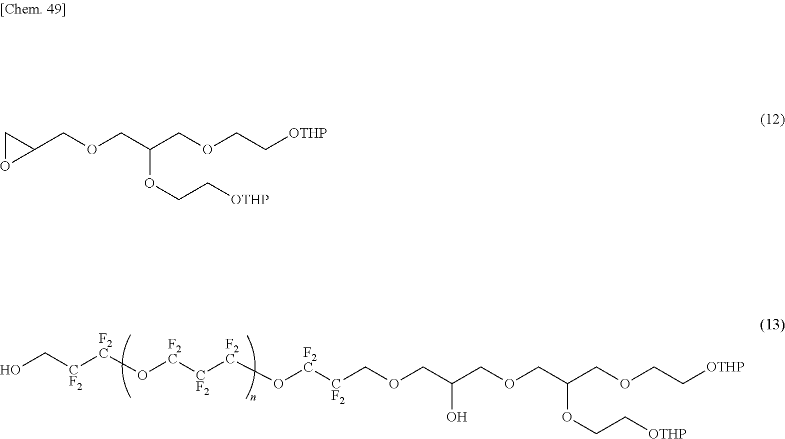

(in Formula (12), THP represents a tetrahydropyranyl group).
(in Formula (13), n indicating an average degree of polymerization represents 3.8, and THP represents a tetrahydropyranyl group).

### (Second Reaction)

11.6 g (a molecular weight of 1,313, 8.8 mmol) of the compound represented by

Formula (13) as the intermediate compound 1-1, 6.0 mL of t-butanol and 0.59 g (a molecular weight of 112.21, 5.3 mmol) of potassium tert-butoxide were put into a 200 mL eggplant flask under a nitrogen gas atmosphere, and stirred at room temperature until they became uniform. 0.50 g of epibromohydrin (a molecular weight of 137, 3.6 mmol) was added to the uniform solution, and the mixture was stirred and reacted at 70° C. for 24 hours.

The reaction solution obtained after the reaction was returned to room temperature, 31 g of a 10% hydrogen chloride/methanol solution (a hydrogen chloride-methanol reagent (5-10%), commercially available from Tokyo Chemical Industry Co., Ltd.) was added, and the mixture was stirred at room temperature for 2 hours. The reaction solution was transferred little by little into a separatory funnel containing 100 mL of a saline solution and extracted three times with 200 mL of ethyl acetate. The organic layer was washed with 100 mL of a saline solution, 100 mL of a saturated sodium bicarbonate solution, and 100 mL of a saline solution in that order, and dehydrated with anhydrous sodium sulfate. After the desiccant was filtered, the filtrate was concentrated, and the residue was purified through silica gel column chromatography.

When the above step was performed, 4.2 g (a number-average molecular weight of 2,349, 1.8 mmol) of a compound (1A) (in Formula (1A), Rf₂1a is represented by Formula (1AF), and in two Rf₂1a's, n1a indicating an average degree of polymerization is 3.8) was obtained.

The obtained compound (1A) was subjected to ¹H-NMR measurement and ¹⁹F-NMR measurement, and the structure was identified based on the following results.

¹H-NMR (CD₃COCD₃): δ [ppm]=3.46 to 4.24 (56H)

¹⁹F-NMR (CD₃COCD₃): δ [ppm]=−84.0 to −83.0 (30.4F), −86.4 (8F), −124.3 (8F), −130.0 to −129.0 (15.2F)

### Example 2

4.3 g (a number-average molecular weight of 2,377, 1.8 mmol) of the compound represented by Formula (1B) (in Formula (1B), Rf₂1b is represented by Formula (1BF), and in two Rf₂1b's, n1b indicating an average degree of polymerization is 3.8) was obtained in the same operation as in Example 1 except that, in the first reaction, in place of the compound represented by Formula (12), 5.5 g (a molecular weight of 419, 13.2 mmol) of a compound represented by the following Formula (14) was used.

The compound represented by Formula (14) was synthesized by the following method. Allyl glycidyl ether was reacted with tetrahydropyranyl ethylene glycol. Synthesis was performed by oxidizing the double bond of the compound obtained by reacting the obtained compound with 2-(3-bromopropoxy)tetrahydro-2H-pyran using m-chloroperbenzoic acid.

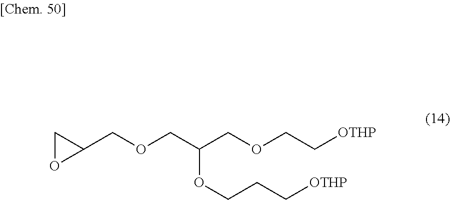

(in Formula (14), THP represents a tetrahydropyranyl group).

The obtained compound (1B) was subjected to ¹H-NMR measurement and ¹⁹F. NMR measurement, and the structure was identified based on the following results.

¹H-NMR (CD₃COCD₃): δ [ppm]=1.65 to 1.81 (4H), 3.39 to 4.35 (56H)

¹⁹F-NMR (CD₃COCD₃): δ [ppm]=−84.0 to −83.0 (30.4F), −86.4 (8F), −124.3 (8F), −130.0 to −129.0 (15.2F)

### Example 3

4.3 g (a number-average molecular weight of 2,399, 1.8 mmol) of the compound represented by Formula (1C) (in Formula (1C), Rf₁1e is represented by Formula (1CF), and in two Rf₁1c's, l1c indicating an average degree of polymerization is 4.0, and m1c indicating an average degree of polymerization is 4.0 was obtained in the same operation as in Example 1 except that, in the first reaction, in place of HOCH₂CF₂CF₂O(CF₂CF₂CF₂O)ₙCF₂CF₂CH₂OH, 20 g of a compound (a number-average molecular weight of 906 and a molecular weight distribution of 1.1) represented by HOCH₂CF₂O(CF₂CF₂O)ₗ(CF₂O)ₘCF₂CH₂OH (in the formula, l indicating an average degree of polymerization is 4.0, and m indicating an average degree of polymerization is 4.0) was used, and in place of the compound represented by Formula (12), 5.7 g (a molecular weight of 432, 13.2 mmol) of a compound represented by the following Formula (15) was used.

The compound represented by Formula (15) was synthesized by the following method. 1 equivalent of 3-allyloxy-1,2-propanediol was reacted with 2 equivalents of 2-(3-bromopropoxy)tetrahydro-2H-pyran. The double bond of the obtained compound was oxidized with m-chloroperbenzoic acid to synthesize the compound represented by Formula (15).

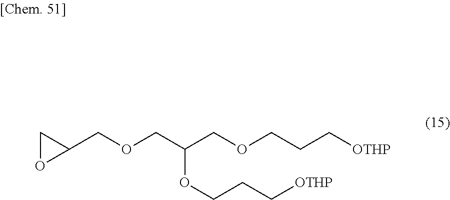

(in Formula (15), THP represents a tetrahydropyranyl group).

The obtained compound (1C) was subjected to ¹H-NMR measurement and ¹⁹F-NMR measurement, and the structure was identified based on the following results.

¹H-NMR (CD₃COCD₃): δ [ppm]=1.65 to 1.81 (8H), 3.39 to 4.35 (56H)

¹⁹F-NMR (CD₃COCD₃): δ [ppm]=−55.6 to −50.6 (16F), −77.7 (4F), −80.3 (4F), −91.0 to −88.5 (32F)

### Example 4

4.4 g (a number-average molecular weight of 2,460, 1.8 mmol) of the compound represented by Formula (1D) (in Formula (1D), Rf₁1d is represented by Formula (1DF), and in two Rf₁1d's, l1d indicating an average degree of polymerization is 6.3 and m1d indicating an average degree of polymerization is 0) was obtained in the same operation as in Example 1 except that, in the first reaction, in place of HOCH₂CF₂CF₂O(CF₂CF₂CF₂O)ₙCF₂CF₂CH₂OH, 20 g of a compound (a number-average molecular weight of 909 and a molecular weight distribution of 1.1) represented by HOCH₂CF₂O(CF₂CF₂O)ₗ(CF₂O)ₘCF₂CH₂OH (in the formula, l indicating an average degree of polymerization is 6.3, and m indicating an average degree of polymerization is 0), and in place of the compound represented by Formula (12), 6.1 g (a molecular weight of 461, 13.2 mmol) of a compound represented by the following Formula (16) was used.

The compound represented by Formula (16) was synthesized by the following method. 1 equivalent of 3-allyloxy-1,2-propanediol was reacted with 2 equivalents of 2-(4-bromobutoxy)tetrahydro-2H-pyran. The double bond of the obtained compound was oxidized with m-chloroperbenzoic acid to synthesize the compound represented by the following Formula (16).

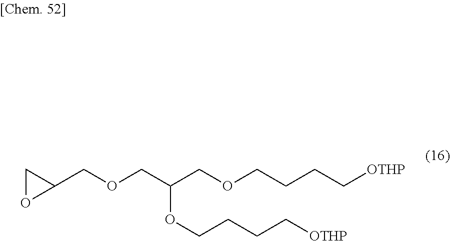

(in Formula (16), THP represents a tetrahydropyranyl group).

The obtained compound (1D) was subjected to ¹H-NMR measurement and ¹⁹F-NMR measurement, and the structure was identified based on the following results.

¹H-NMR (CD₃COCD₃): δ [ppm]=1.65 to 1.81 (16H), 3.39 to 4.35 (56H)

¹⁹F-NMR (CD₃COCD₃): δ [ppm]=−78.6 (4F), −81.3 (4F), −90.0 to −88.5 (50.4F)

### Example 5

4.3 g (a number-average molecular weight of 2,405, 1.8 mmol) of the compound represented by Formula (1E) (in Formula (1E), Rf₂1e is represented by Formula (1EF), and in two Rf₂1e's, n1e indicating an average degree of polymerization is 3.8) was obtained in the same operation as in Example 1 except that, in the first reaction, in place of the compound represented by Formula (12), 5.7 g (a molecular weight of 432, 13.2 mmol) of the compound represented by the following Formula (18) was used.

The compound represented by Formula (18) was synthesized by the following method. The double bond of di(3-butenyl) ether on one side was oxidized using 1 equivalent of m-chloroperbenzoic acid to form an epoxy group, the epoxy group was then ring-opened using concentrated sulfuric acid to synthesize the compound represented by the following Formula (17). The obtained compound represented by Formula (17) was reacted with 2 equivalents of 2-(2-bromoethoxy)tetrahydro-2H-pyran. Then, the double bond was oxidized using m-chloroperbenzoic acid to synthesize the compound represented by Formula (18).

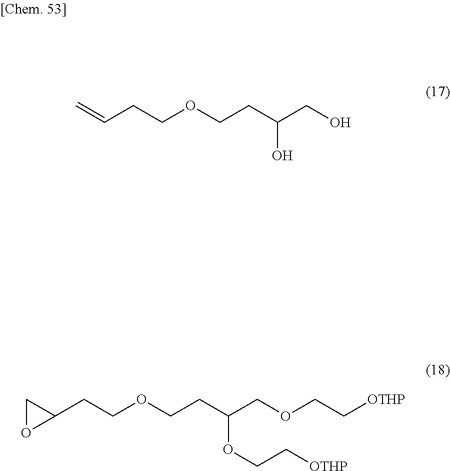

(in Formula (18), THP represents a tetrahydropyranyl group).

The obtained compound (1E) was subjected to ¹H-NMR measurement and ¹⁹F-NMR measurement, and the structure was identified based on the following results.

¹H-NMR (CD₃COCD₃): δ [ppm]=1.65 to 1.81 (8H), 3.39 to 4.35 (56H)

¹⁹F-NMR (CD₃COCD₃): δ [ppm]=−84.0 to −83.0 (30.4F), −86.4 (8F), −124.3 (8F), −130.0 to −129.0 (15.2F)

### Example 6

4.6 g (a number-average molecular weight of 2,573, 1.8 mmol) of the compound represented by Formula (1F) (in Formula (1F), Rf₂1f is represented by Formula (1FF), and in two Rf₂1f's, n1f indicating an average degree of polymerization is 3.8) was obtained in the same operation as in Example 1 except that, in the first reaction, in place of the compound represented by Formula (12), 6.8 g (a molecular weight of 516, 13.2 mmol) of a compound represented by the following Formula (20) was used.

The compound represented by Formula (20) was synthesized by the following method. The double bond of di(6-heptenyl) ether on one side was oxidized using 1 equivalent of m-chloroperbenzoic acid to form an epoxy group, and the epoxy group was then ring-opened using concentrated sulfuric acid to synthesize a compound represented by the following Formula (19). The obtained compound represented by Formula (19) was reacted with 2 equivalents of 2-(2-bromoethoxy)tetrahydro-2H-pyran. Then, oxidation was performed using m-chloroperbenzoic acid to synthesize a compound represented by the following Formula (20).

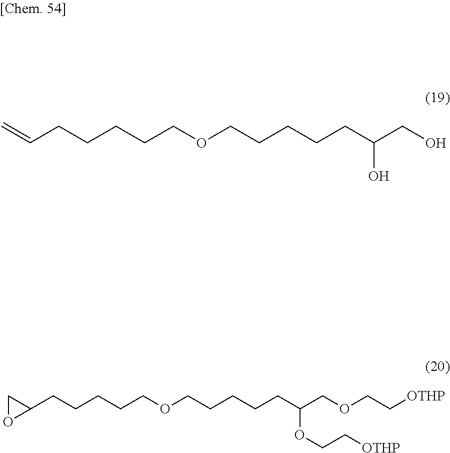

(in Formula (20), THP represents a tetrahydropyranyl group).

The obtained compound (1F) was subjected to ¹H-NMR measurement and ¹⁹F-NMR measurement, and the structure was identified based on the following results.

¹H-NMR (CD₃COCD₃): δ [ppm]=1.65 to 1.81 (32H), 3.39 to 4.35 (56H)

¹⁹F-NMR (CD₃COCD₃): δ [ppm]=−84.0 to −83.0 (30.4F), −86.4 (8F), −124.3 (8F), −130.0 to −129.0 (15.2F)

### Example 7

4.5 g (a number-average molecular weight of 2,497, 1.8 mmol) of the compound represented by Formula (1G) (in Formula (1G), Rf₂1g is represented by Formula (1GF), and in two Rf₂1g's, n1g indicating an average degree of polymerization is 3.8) was obtained in the same operation as in Example 1 except that, in the first reaction, in place of the compound represented by Formula (12), 6.3 g (a molecular weight of 479, 13.2 mmol) of a compound represented by the following Formula (21) was obtained.

The compound represented by the following Formula (21) was synthesized by reacting the compound represented by Formula (12) with allyl alcohol and then oxidizing the double bond of the obtained compound with m-chloroperbenzoic acid.

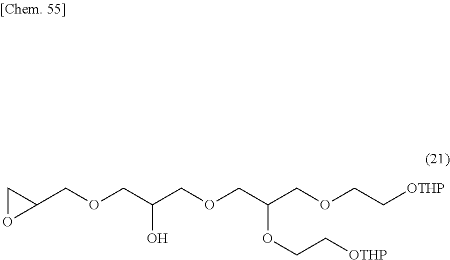

(in Formula (21), THP represents a tetrahydropyranyl group).

The obtained compound (1G) was subjected to ¹H-NMR measurement and ¹⁹F-NMR measurement, and the structure was identified based on the following results.

¹H-NMR (CD₃COCD₃): δ [ppm]=3.39 to 4.35 (68H)

¹⁹F-NMR (CD₃COCD₃): δ [ppm]=−84.0 to −83.0 (30.4F), −86.4 (8F), −124.3 (8F), −130.0 to −129.0 (15.2F)

### Example 8

3.9 g (a number-average molecular weight of 2,167, 1.8 mmol) of the compound represented by Formula (1H) (in Formula (1H), Rf₁1h is represented by Formula (1HF), and in two Rf₁1h's, l1h indicating an average degree of polymerization is 4.0 and m1h indicating an average degree of polymerization is 4.0) was obtained in the same operation as in Example 3 except that, in the first reaction, in place of the compound represented by Formula (15), 2.5 g (a molecular weight of 188, 13.2 mmol) of a compound represented by the following Formula (23) was used.

The compound represented by the following Formula (23) was synthesized by the following method. The compound represented by the following Formula (22) was synthesized by reducing carbonyl moieties of 2,2-dimethyl-1,3-dioxan-5-one with lithium aluminum hydride. The obtained compound represented by Formula (22) was reacted with epibromohydrin to synthesize a compound represented by the following Formula (23).

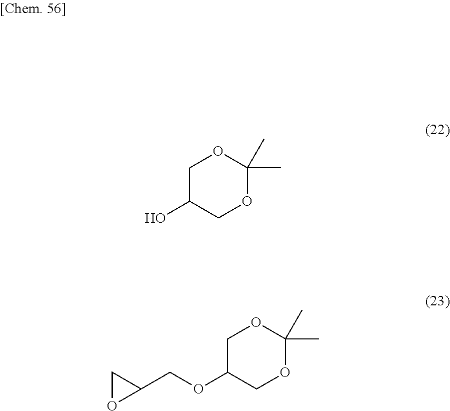

The obtained compound (1H) was subjected to ¹H-NMR measurement and ¹⁹F-NMR measurement, and the structure was identified based on the following results.

¹H-NMR (CD₃COCD₃): δ [ppm]=3.39 to 4.35 (40H)

¹⁹F-NMR (CD₃COCD₃): δ [ppm]=−84.0 to −83.0 (30.4F), −86.4 (8F), −124.3 (8F), −130.0 to −129.0 (15.2F)

### Example 9

4.0 g (a number-average molecular weight of 2,200, 1.8 mmol) of the compound represented by Formula (11) (in Formula (11), Rf₁1i is represented by Formula (11F), and in two Rf₁1i's, l1i indicating an average degree of polymerization is 6.3 and m1i indicating an average degree of polymerization is 0) was obtained in the same operation as in Example 4 except that, in the first reaction, in place of the compound represented by

Formula (16), 2.7 g (a molecular weight of 202, 13.2 mmol) of a compound represented by the following Formula (24) was used.

The compound represented by Formula (24) was synthesized by reacting 5-hydroxymethyl-2,2-dimethyl-1,3-dioxane with epibromohydrin.

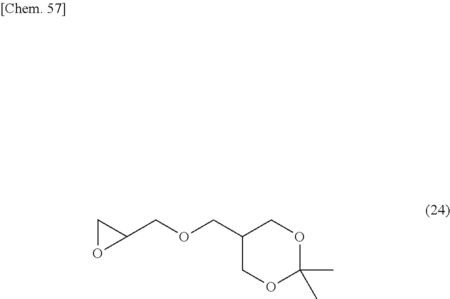

The obtained compound (11) was subjected to ¹H-NMR measurement and ¹⁹F-NMR measurement, and the structure was identified based on the following results.

¹H-NMR (CD₃COCD₃): δ [ppm]=1.65 to 1.81 (2H), 3.39 to 4.35 (52H)

¹⁹F-NMR (CD₃COCD₃): δ [ppm]=−78.6 (4F), −81.3 (4F), −90.0 to −88.5 (50.4F)

### Example 10

4.0 g (a number-average molecular weight of 2,228, 1.8 mmol) of the compound represented by Formula (1J) (in Formula (1J), Rf₂1j is represented by Formula (1JF), and in two Rf₂1j's, n1j indicating an average degree of polymerization is 3.8) was obtained in the same operation as in Example 1 except that, in the first reaction, in place of the compound represented by Formula (12), 2.9 g (a molecular weight of 216, 13.2 mmol) of a compound represented by the following Formula (25) was used.

The compound represented by Formula (25) was synthesized by reacting 5-hydroxyethyl-2,2-dimethyl-1,3-dioxane with epibromohydrin.

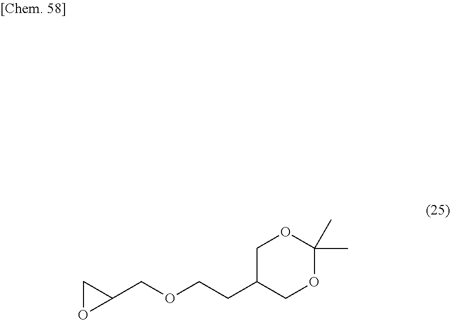

The obtained compound (1J) was subjected to ¹H-NMR measurement and ¹⁹F-NMR measurement, and the structure was identified based on the following results.

¹H-NMR (CD₃COCD₃): δ [ppm]=1.65 to 1.81 (6H), 3.39 to 4.35 (42H)

¹⁹F-NMR (CD₃COCD₃): δ [ppm]=−84.0 to −83.0 (30.4F), −86.4 (8F), −124.3 (8F), −130.0 to −129.0 (15.2F)

### Example 11

4.2 g (a number-average molecular weight of 2,340, 1.8 mmol) of the compound represented by Formula (1K) (in Formula (1K), Rf₁1k is represented by Formula (1KF), and in two Rf₁1k's, l1k indicating an average degree of polymerization is 6.3 and m1k indicating an average degree of polymerization is 0) was obtained in the same operation as in Example 4 except that, in the first reaction, in place of the compound represented by Formula (16), 3.6 g (a molecular weight of 272, 13.2 mmol) of a compound represented by the following Formula (26) was used.

The compound represented by Formula (26) was synthesized by reacting 5-hydroxyhexyl-2,2-dimethyl-1,3-dioxane with epibromohydrin.

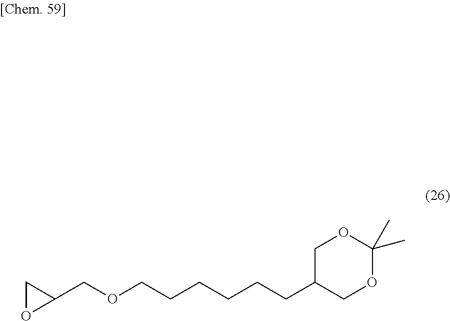 [SMILES: CC1(C)OCC(CCCCCCOCC2CO2)CO1]

The obtained compound (1K) was subjected to ¹H-NMR measurement and 1° F.

NMR measurement, and the structure was identified based on the following results.

¹H-NMR (CD₃COCD₃): δ [ppm]=1.65 to 1.81 (22H), 3.39 to 4.35 (42H)

¹⁹F-NMR (CD₃COCD₃): δ [ppm]=−78.6 (4F), −81.3 (4F), −90.0 to −88.5 (50.4F)

### Example 12

4.0 g (a number-average molecular weight of 2,200, 1.8 mmol) of the compound represented by Formula (1L) (in Formula (1L), Rf₂1l is represented by Formula (1LF), and in two Rf₂1l's, n1l indicating an average degree of polymerization is 3.8) was obtained in the same operation as in Example 1 except that, in the first reaction, in place of the compound represented by Formula (12), 2.7 g (a molecular weight of 202, 13.2 mmol) of a compound represented by the following Formula (27) was used.

The compound represented by Formula (27) was synthesized by reacting the compound represented by Formula (22) with 3-butenyl bromide and then oxidizing the double bond of the obtained compound with m-chloroperbenzoic acid.

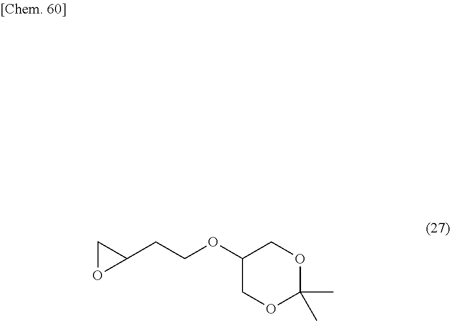

The obtained compound (1L) was subjected to ¹H-NMR measurement and ¹⁹F-NMR measurement, and the structure was identified based on the following results.

¹H-NMR (CD₃COCD₃): δ [ppm]=1.65 to 1.81 (4H), 3.39 to 4.35 (40H)

¹⁹F-NMR (CD₃COCD₃): δ [ppm]=−84.0 to −83.0 (30.4F), −86.4 (8F), −124.3 (8F), −130.0 to −129.0 (15.2F)

### Example 13

4.2 g (a number-average molecular weight of 2,349, 1.8 mmol) of the compound represented by Formula (1M) (in Formula (1M), Rf₂1m is represented by Formula (1MF), and in two Rf₂1m's, n1m indicating an average degree of polymerization is 3.8) was obtained in the same operation as in Example 1 except that, in the first reaction, in place of the compound represented by Formula (12), 3.6 g (a molecular weight of 276, 13.2 mmol) of a compound represented by the following Formula (28) was used.

The compound represented by Formula (28) was synthesized by reacting the compound represented by Formula (23) with 3-buten-1-ol and then oxidizing the double bond of the obtained compound with m-chloroperbenzoic acid.

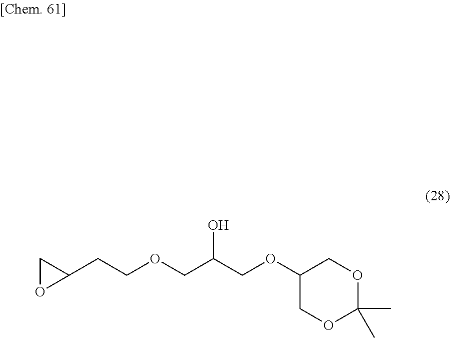 [SMILES: CC1(C)OCC(OCC(O)COCCC2CO2)CO1]

The obtained compound (1M) was subjected to ¹H-NMR measurement and ¹⁹F-NMR measurement, and the structure was identified based on the following results.

¹H-NMR (CD₃COCD₃): δ [ppm]=1.65 to 1.81 (4H), 3.39 to 4.35 (52H)

¹⁹F-NMR (CD₃COCD₃): δ [ppm]=−84.0 to −83.0 (30.4F), −86.4 (8F), −124.3 (8F), −130.0 to −129.0 (15.2F)

### Example 14

4.2 g (a number-average molecular weight of 2,321, 1.8 mmol) of the compound represented by Formula (1N) (in Formula (1N), Rf₂1n is represented by Formula (1NF), and in two Rf₂1n's, n1n indicating an average degree of polymerization is 3.8) was obtained in the same operation as in Example 1 except that, in the first reaction, in place of the compound represented by Formula (12), 5.3 g (a molecular weight of 405, 13.2 mmol) of a compound represented by the following Formula (29) was used.

The compound represented by Formula (29) was synthesized by reacting the compound represented by Formula (23) with allyl alcohol and then oxidizing the double bond of the obtained compound with m-chloroperbenzoic acid.

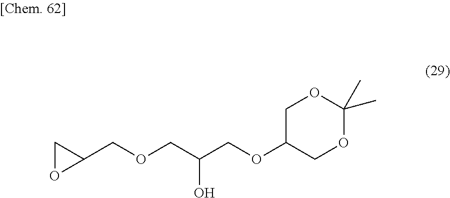 [SMILES: CC1(C)OCC(OCC(O)COCC2CO2)CO1]

The obtained compound (1N) was subjected to ¹H-NMR measurement and ¹⁹F-NMR measurement, and the structure was identified based on the following results.

¹H-NMR (CD₃COCD₃): δ [ppm]=3.39 to 4.35 (52H)

¹⁹F-NMR (CD₃COCD₃): δ [ppm]=−84.0 to −83.0 (30.4F), −86.4 (8F), −124.3 (8F), −130.0 to −129.0 (15.2F)

### Example 15

4.2 g (a number-average molecular weight of 2,349, 1.8 mmol) of the compound represented by Formula (10) (in Formula (10), Rf₂1o is represented by Formula (1° F.), and in two Rf₂1o's, n1o indicating an average degree of polymerization is 3.8) was obtained in the same operation as in Example 1 except that, in the first reaction, in place of the compound represented by Formula (12), a 5.3 g (a molecular weight of 404, 13.2 mmol) of a compound represented by the following Formula (31) was used.

The compound represented by Formula (31) was synthesized by the following method. 2 equivalents of tetrahydropyranyl ethylene glycol were reacted with 1 equivalent of epichlorohydrin to synthesize a compound represented by the following Formula (30). The obtained compound represented by Formula (30) was reacted with epibromohydrin to synthesize a compound represented by the following Formula (31).

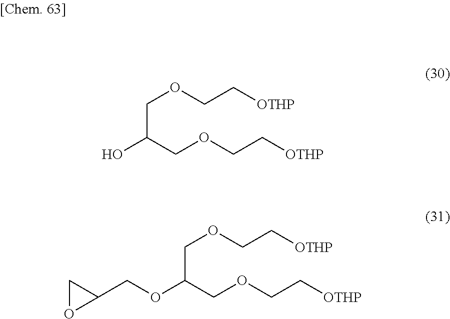

(in Formula (30), THP represents a tetrahydropyranyl group).
(in Formula (31), THP represents a tetrahydropyranyl group).

The obtained compound (10) was subjected to ¹H-NMR measurement and ¹⁹F-NMR measurement, and the structure was identified based on the following results.

¹H-NMR (CD₃COCD₃): δ [ppm]=3.39 to 4.35 (56H)

¹⁹F-NMR (CD₃COCD₃): δ [ppm]=−84.0 to −83.0 (30.4F), −86.4 (8F), −124.3 (8F), −130.0 to −129.0 (15.2F)

### Example 16

4.3 g (a number-average molecular weight of 2,405, 1.8 mmol) of the compound represented by Formula (1P) (in Formula (1P), Rf₂1p is represented by Formula (1 PF), and in two Rf₂1p's, n1p indicating an average degree of polymerization is 3.8) was obtained in the same operation as in Example 1 except that, in the first reaction, in place of the compound represented by Formula (12), 5.7 g (a molecular weight of 433, 13.2 mmol) of a compound represented by the following Formula (33) was used.

The compound represented by Formula (33) was synthesized by the following method. 2 equivalents of tetrahydropyranyl trimethylene glycol were reacted with 1 equivalent of epichlorohydrin to synthesize a compound represented by the following Formula (32). The obtained compound represented by Formula (32) was reacted with epibromohydrin to synthesize a compound represented by the following Formula (33).

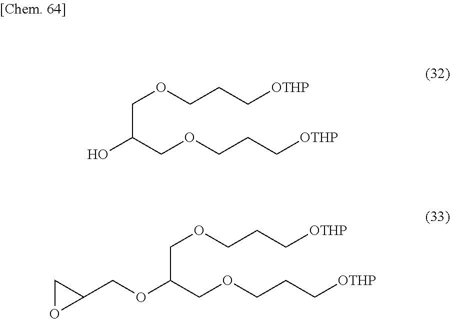

(in Formula (32), THP represents a tetrahydropyranyl group).
(in Formula (33), THP represents a tetrahydropyranyl group).

The obtained compound (1P) was subjected to ¹H-NMR measurement and ¹⁹F-NMR measurement, and the structure was identified based on the following results.

¹H-NMR (CD₃COCD₃): δ [ppm]=1.65 to 1.81 (10H), 3.39 to 4.35 (54H)

¹⁹F-NMR (CD₃COCD₃): δ [ppm]=−84.0 to −83.0 (30.4F), −86.4 (8F), −124.3 (8F), −130.0 to −129.0 (15.2F)

### Example 17

4.4 g (a number-average molecular weight of 2,455, 1.8 mmol) of the compound represented by Formula (1Q) (in Formula (1Q), Rf₁1q is represented by Formula (1QF), and in two Rf₁1q's, l1q indicating an average degree of polymerization is 4.0 and m1q indicating an average degree of polymerization is 4.0) was obtained in the same operation as in Example 3 except that, in the first reaction, in place of the compound represented by Formula (15), 6.1 g (a molecular weight of 461, 13.2 mmol) of a compound represented by the following Formula (35) was used.

The compound represented by Formula (35) was synthesized by the following method. 2 equivalents of tetrahydropyranyl tetramethylene glycol were reacted with 1 equivalent of epichlorohydrin to synthesize a compound represented by the following Formula (34). The obtained compound represented by Formula (34) was reacted with epibromohydrin to synthesize a compound represented by the following Formula (35).

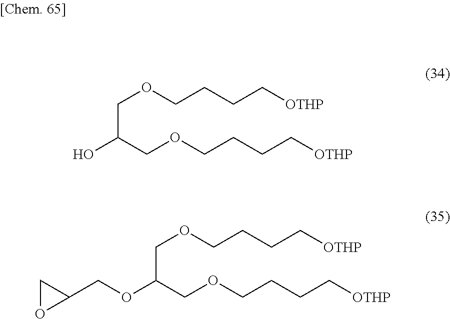

(in Formula (34), THP represents a tetrahydropyranyl group).
(in Formula (35), THP represents a tetrahydropyranyl group).

The obtained compound (1Q) was subjected to ¹H-NMR measurement and ¹⁹F-NMR measurement, and the structure was identified based on the following results.

¹H-NMR (CD₃COCD₃): δ [ppm]=1.65 to 1.81 (18H), 3.39 to 4.35 (54H)

¹⁹F-NMR (CD₃COCD₃): δ [ppm]=−84.0 to −83.0 (30.4F), −86.4 (8F), −124.3 (8F), −130.0 to −129.0 (15.2F)

### Example 18

4.6 g (a number-average molecular weight of 2,573, 1.8 mmol) of the compound represented by Formula (1R) (in Formula (1R), Rf₁1r is represented by Formula (1RF), and in two Rf₁1r's, l1r indicating an average degree of polymerization is 6.3 and m1r indicating an average degree of polymerization is 0) was obtained in the same operation as in Example 4 except that, in the first reaction, in place of the compound represented by Formula (16), 6.8 g (a molecular weight of 517, 13.2 mmol) of a compound represented by the following Formula (37) was used.

The compound represented by Formula (37) was synthesized by the following method. 2 equivalents of tetrahydropyranyl hexamethylene glycol were reacted with 1 equivalent of epichlorohydrin to synthesize a compound represented by the following Formula (36). The obtained compound represented by Formula (36) was reacted with epibromohydrin to synthesize a compound represented by the following Formula (37).

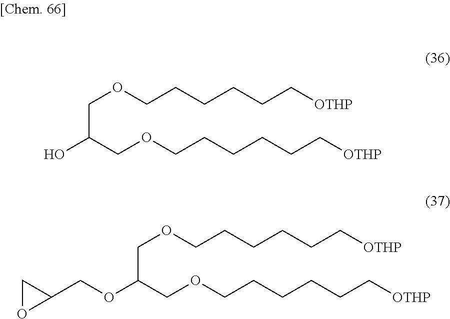

(in Formula (36), THP represents a tetrahydropyranyl group).
(in Formula (37), THP represents a tetrahydropyranyl group).

The obtained compound (1R) was subjected to ¹H-NMR measurement and ¹⁹F-NMR measurement, and the structure was identified based on the following results.

¹H-NMR (CD₃COCD₃): δ [ppm]=1.65 to 1.81 (32H), 3.39 to 4.35 (56H)

¹⁹F-NMR (CD₃COCD₃): δ [ppm]=−78.6 (4F), −81.3 (4F), −90.0 to −88.5 (50.4F)

### Example 19

4.7 g (a number-average molecular weight of 2,595, 1.8 mmol) of the compound represented by Formula (1S) (in Formula (1S), Rf₁1s is represented by Formula (1SF), and in two Rf₁1s's, l1s indicating an average degree of polymerization is 4.0 and m1s indicating an average degree of polymerization is 4.0) was obtained in the same operation as in Example 3 except that, in the first reaction, in place of the compound represented by Formula (15), 7.0 g (a molecular weight of 531, 13.2 mmol) of a compound represented by the following Formula (39) was used.

The compound represented by Formula (39) was synthesized by the following method. 1 equivalent of 4-allyloxy-1,2-butanediol was reacted with 2 equivalents of 2-(6-bromohexyloxy)tetrahydro-2H-pyran to synthesize a compound represented by the following Formula (38). The double bond of the obtained compound represented by

Formula (38) was oxidized with m-chloroperbenzoic acid to synthesize a compound represented by the following Formula (39).

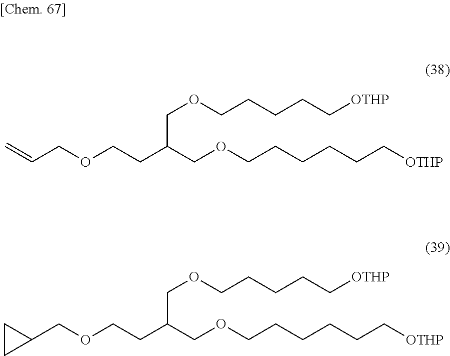

(in Formula (38), THP represents a tetrahydropyranyl group).
(in Formula (39), THP represents a tetrahydropyranyl group).

The obtained compound (1S) was subjected to ¹H-NMR measurement and ¹⁹F-NMR measurement, and the structure was identified based on the following results.

¹H-NMR (CD₃COCD₃): δ [ppm]=1.65 to 1.81 (36H), 3.39 to 4.35 (56H)

¹⁹F-NMR (CD₃COCD₃): δ [ppm]=−84.0 to −83.0 (30.4F), −86.4 (8F), −124.3 (8F), −130.0 to −129.0 (15.2F)

### Example 20

4.9 g (a number-average molecular weight of 2,713, 1.8 mmol) of the compound represented by Formula (1T) (in Formula (1T), Rf₁1t is represented by Formula (1TF), and in two Rf₁1t's, l1t indicating an average degree of polymerization is 6.3 and m1t indicating an average degree of polymerization is 0) was obtained in the same operation as in Example 4 except that, in the first reaction, in place of the compound represented by Formula (16), 7.7 g (a molecular weight of 587, 13.2 mmol) of a compound represented by the following Formula (41) was used.

The compound represented by Formula (41) was synthesized by the following method. 1 equivalent of 8-allyloxy-1,2-octanediol was reacted with 2 equivalents of 2-(6-bromohexyloxy)tetrahydro-2H-pyran to synthesize a compound represented by the following Formula (40). The double bond of the obtained compound represented by Formula (40) was oxidized with m-chloroperbenzoic acid to synthesize a compound represented by the following Formula (41).

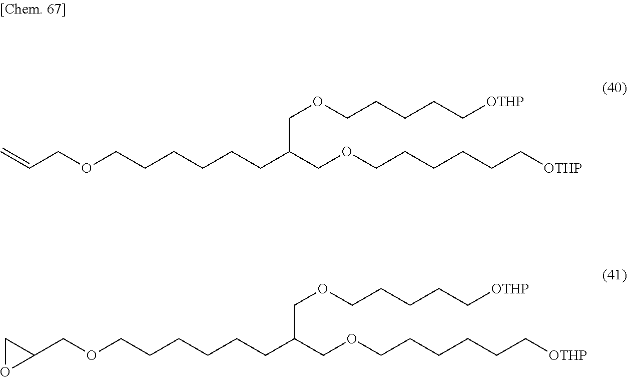

(in Formula (40), THP represents a tetrahydropyranyl group).
(in Formula (41), THP represents a tetrahydropyranyl group).

The obtained compound (1T) was subjected to ¹H-NMR measurement and ¹⁹F-NMR measurement, and the structure was identified based on the following results.

¹H-NMR (CD₃COCD₃): δ [ppm]=1.65 to 1.81 (52H), 3.39 to 4.35 (56H)

¹⁹F-NMR (CD₃COCD₃): δ [ppm]=−78.6 (4F), −81.3 (4F), −90.0 to −88.5 (50.4F)

### Example 21

4.5 g (a number-average molecular weight of 2,525, 1.8 mmol) of the compound represented by Formula (1U) (in Formula (1U), Rf₂1u is represented by Formula (1UF), and in two Rf₂1u's, n1u indicating an average degree of polymerization is 3.8) was obtained in the same operation as in Example 1 except that, in the first reaction, in place of the compound represented by Formula (12), 6.5 g (a molecular weight of 493, 13.2 mmol) of a compound represented by the following Formula (43) was used.

The compound represented by Formula (43) was synthesized by the following method. 2 equivalents of tetrahydropyranyl diethylene glycol were reacted with 1 equivalent of epichlorohydrin to synthesize a compound represented by the following Formula (42). The obtained compound represented by Formula (42) was reacted with epibromohydrin to synthesize a compound represented by the following Formula (43).

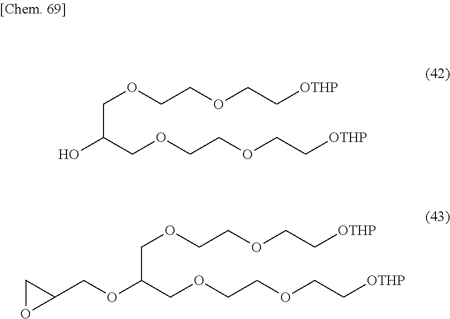

(in Formula (42), THP represents a tetrahydropyranyl group).
(in Formula (43), THP represents a tetrahydropyranyl group).

The obtained compound (1U) was subjected to ¹H-NMR measurement and ¹⁹F-NMR measurement, and the structure was identified based on the following results.

¹H-NMR (CD₃COCD₃): δ [ppm]=3.39 to 4.35 (72H)

¹⁹F-NMR (CD₃COCD₃): δ [ppm]=−84.0 to −83.0 (30.4F), −86.4 (8F), −124.3 (8F), −130.0 to −129.0 (15.2F)

### Example 22

4.1 g (a number-average molecular weight of 2,260, 1.8 mmol) of the compound represented by Formula (1V) (in Formula (1V), Rf₂1v is represented by Formula (1VF), and in two Rf₂1v's, n1v indicating an average degree of polymerization is 3.8) was obtained in the same operation as in Example 1 except that, in the first reaction, in place of the compound represented by Formula (12), a 5.9 g (a molecular weight of 445, 13.2 mmol) of a compound represented by the following Formula (45) was used.

The compound represented by Formula (45) was synthesized by the following method. 2-(bromomethyl)-2-(hydroxymethyl)-1,3-propanediol was reacted with 3,4-dihydro-2H-pyran, and a hydroxy group of 2-(bromomethyl)-2-(hydroxymethyl)-1,3-propanediol was protected with a tetrahydropyranyl group to synthesize a compound represented by the following Formula (44). The obtained compound represented by Formula (44) was reacted with allyl alcohol, the double bond of the obtained compound was then oxidized with m-chloroperbenzoic acid to synthesize a compound represented by the following Formula (45).

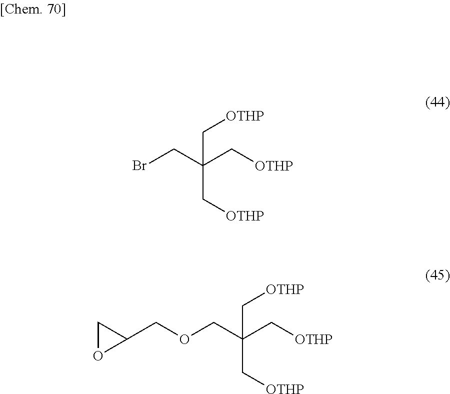

(in Formula (44), THP represents a tetrahydropyranyl group).
(in Formula (45), THP represents a tetrahydropyranyl group).

The obtained compound (1V) was subjected to ¹H-NMR measurement and ¹⁹F-NMR measurement, and the structure was identified based on the following results.

¹H-NMR (CD₃COCD₃): δ [ppm]=3.39 to 4.35 (48H)

¹⁹F-NMR (CD₃COCD₃): δ [ppm]=−84.0 to −83.0 (30.4F), −86.4 (8F), −124.3 (8F), −130.0 to −129.0 (15.2F)

### Example 23

The compound represented by Formula (2A) was produced by the following method.

### (First Reaction)

12.2 g (20 mmol) of a compound (a number-average molecular weight of 610 and a molecular weight distribution of 1.1) represented by HOCH₂CF₂CF₂O(CF₂CF₂CF₂O)ₙCF₂CF₂CH₂OH (in the formula, n indicating an average degree of polymerization is 2.0), 1.76 g (44 mmol) of 60% sodium hydride, and 15.6 mL of N,N-dimethylformamide were put into a 200 mL eggplant flask under a nitrogen gas atmosphere, and stirred at room temperature until they became uniform. 3.45 mL of epibromohydrin (42 mmol) was added to the uniform solution, and the mixture was stirred and reacted at 40° C. for 2 hours.

The reaction product obtained after the reaction was cooled to 25° C., 80 mL of water was added to stop the reaction, and the mixture was transferred into a separatory funnel and extracted twice with 150 mL of ethyl acetate. The organic layer was washed with a saturated saline and dehydrated with anhydrous sodium sulfate. After the desiccant was filtered, the filtrate was concentrated, and the residue was purified through silica gel column chromatography to obtain 3.5 g (a molecular weight of 580, 6.0 mmol) of a compound represented by the following Formula (46) as an intermediate compound 2-1.

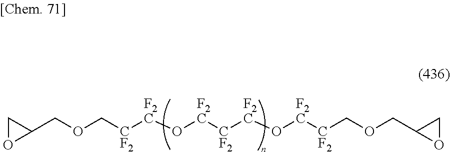

(in Formula (46), n indicating an average degree of polymerization is 2.0).

### (Second Reaction)

14 g of a compound (a number-average molecular weight of 610 and a molecular weight distribution of 1.1) represented by HOCH₂CF₂CF₂O(CF₂CF₂CF₂O)ₙCF₂CF₂CH₂OH (in the formula, n indicating an average degree of polymerization is 2.0), 5.4 g (a molecular weight of 405, 13 mmol) of the compound represented by Formula (12), and 20 mL of t-butanol were put into a 200 mL eggplant flask under a nitrogen gas atmosphere, and stirred at room temperature until they became uniform. 0.74 g of potassium tert-butoxide (a molecular weight of 112, 6.6 mmol) was additionally added to the uniform solution, and the mixture was stirred and reacted at 70° C. for 16 hours.

The reaction product obtained after the reaction was cooled to 25° C., transferred into a separatory funnel containing 100 mL of water, and extracted three times with 100 mL of ethyl acetate. The organic layer was washed with water and dehydrated with anhydrous sodium sulfate. After the desiccant was filtered, the filtrate was concentrated, and the residue was purified through silica gel column chromatography to obtain 12 g (a molecular weight of 1,015, 12.0 mmol) of the compound represented by Formula (13)
(in Formula (13), n indicating an average degree of polymerization represents 2.0, and THP represents a tetrahydropyranyl group) as an intermediate compound 2-2.

### (Third Reaction)

10 g of the intermediate compound 2-2 represented by Formula (13) (in the formula, n indicating an average degree of polymerization is 2.0), 0.38 g of potassium tert-butoxide, and 9.5 mL of t-butanol were put into a 200 mL eggplant flask under a nitrogen gas atmosphere, and stirred at room temperature until they became uniform. 2.2 g of the intermediate compound 2-1 represented by Formula (46) (in the formula, n indicating an average degree of polymerization is 2.0) was added to the uniform solution, and the mixture was stirred and reacted at 70° C. for 16 hours.

The reaction product obtained after the reaction was cooled to 25° C., transferred into a separatory funnel containing 100 mL of water, and extracted three times with 100 mL of ethyl acetate. The organic layer was washed with water and dehydrated with anhydrous sodium sulfate. After the desiccant was filtered, the filtrate was concentrated, and the residue was purified through silica gel column chromatography to obtain 4.4 g (a molecular weight of 2,419, 1.8 mmol) of the compound represented by Formula (2A) (in Formula (2A), Rf₂2a is represented by Formula (2AF), and in three Rf₂2a's, n2a indicating an average degree of polymerization is 2.0).

The obtained compound (2A) was subjected to ¹H-NMR measurement and ¹⁹F-NMR measurement, and the structure was identified based on the following results.

¹H-NMR (CD₃COCD₃): δ [ppm]=3.46 to 4.24 (66H)

¹⁹F-NMR (CD₃COCD₃): δ [ppm]=−84.0 to −83.0 (24F), −86.4 (12F), −124.3 (12F), −130.0 to −129.0 (12F)

### Example 24

4.4 g (a molecular weight of 2,447, 1.8 mmol) of the compound represented by Formula (2B) (in Formula (2B), Rf₂2b is represented by Formula (2BF), and in three Rf₂2b's, n2b indicating an average degree of polymerization is 2.0) was obtained in the same operation as in Example 23 except that, in the second reaction, in place of the compound represented by Formula (12), 5.6 g of the compound represented by Formula (14) was used.

The obtained compound (2B) was subjected to ¹H-NMR measurement and ¹⁹F-NMR measurement, and the structure was identified based on the following results.

¹H-NMR (CD₃COCD₃): δ [ppm]=1.65 to 1.81 (4H), 3.39 to 4.35 (66H)

¹⁹F-NMR (CD₃COCD₃): δ [ppm]=−84.0 to −83.0 (24F), −86.4 (12F), −124.3 (12F), −130.0 to −129.0 (12F)

### Example 25

4.5 g (a number-average molecular weight of 2,490, 1.8 mmol) of the compound represented by Formula (2C) (in Formula (2C), Rf₁2c is represented by Formula (2CF), and in three Rf₁2c's, l2c indicating an average degree of polymerization is 2.4 and m2c indicating an average degree of polymerization is 2.4) was obtained in the same operation as in Example 23 except that, in the first reaction and the second reaction, in place of HOCH₂CF₂CF₂O(CF₂CF₂CF₂O)ₙCF₂CF₂CH₂OH, 12 g of a compound (a number-average molecular weight of 615 and a molecular weight distribution of 1.1) represented by HOCH₂CF₂O(CF₂CF₂O)ₗ(CF₂O)ₘCF₂CH₂OH (in the formula, l indicating an average degree of polymerization is 2.4, and m indicating an average degree of polymerization is 2.4) was used, and in the second reaction, in place of the compound represented by Formula (12), 5.7 g of the compound represented by Formula (15) was used.

The obtained compound (2C) was subjected to ¹H-NMR measurement and ¹⁹F-NMR measurement, and the structure was identified based on the following results.

¹H-NMR (CD₃COCD₃): δ [ppm]=1.65 to 1.81 (8H), 3.39 to 4.35 (66H)

¹⁹F-NMR (CD₃COCD₃): δ [ppm]=−55.6 to −50.6 (14.4F), −77.7 (6F), −80.3 (6F), −91.0 to −88.5 (28.8F)

### Example 26

4.6 g (a number-average molecular weight of 2,557, 1.8 mmol) of the compound represented by Formula (2D) (in Formula (2D), Rf₁2d is represented by Formula (2DF), and in three Rf₁2d's, l2d indicating an average degree of polymerization is 3.8 and m2d indicating an average degree of polymerization is 0) was obtained in the same operation as in Example 23 except that, in the first reaction and the second reaction, in place of HOCH₂CF₂CF₂O(CF₂CF₂CF₂O)ₙCF₂CF₂CH₂OH, 12 g of a compound (a number-average molecular weight of 619 and a molecular weight distribution of 1.1) represented by HOCH₂CF₂O(CF₂CF₂O)ₗ(CF₂O)ₘCF₂CH₂OH (in the formula, l indicating an average degree of polymerization is 3.8, and m indicating an average degree of polymerization is 0) was used, and in the second reaction, in place of the compound represented by Formula (12), 6.0 g of the compound represented by Formula (16) was used.

The obtained compound (2D) was subjected to ¹H-NMR measurement and ¹⁹F-NMR measurement, and the structure was identified based on the following results.

¹H-NMR (CD₃COCD₃): δ [ppm]=1.65 to 1.81 (16H), 3.39 to 4.35 (66H)

¹⁹F-NMR (CD₃COCD₃): δ [ppm]=−78.6 (6F), −81.3 (6F), −90.0 to −88.5 (45.6F)

### Example 27

4.5 g (a molecular weight of 2,475, 1.8 mmol) of the compound represented by Formula (2E) (in Formula (2E), Rf₂2e is represented by Formula (2EF), and in three Rf₂2e's, n2e indicating an average degree of polymerization is 2.0) was obtained in the same operation as in Example 23 except that, in the second reaction, in place of the compound represented by Formula (12), 5.7 g of the compound represented by Formula (18) was used.

The obtained compound (2E) was subjected to ¹H-NMR measurement and ¹⁹F-NMR measurement, and the structure was identified based on the following results.

¹H-NMR (CD₃COCD₃): δ [ppm]=1.65 to 1.81 (8H), 3.39 to 4.35 (66H)

¹⁹F-NMR (CD₃COCD₃): δ [ppm]=−84.0 to −83.0 (24F), −86.4 (12F), −124.3 (12F), −130.0 to −129.0 (12F)

### Example 28

4.8 g (a molecular weight of 2,644, 1.8 mmol) of the compound represented by Formula (2F) (in Formula (2F), Rf₂2f is represented by Formula (2FF), and in three Rf₂2f's, n2f indicating an average degree of polymerization is 2.0) was obtained in the same operation as in Example 23 except that, in the second reaction, in place of the compound represented by Formula (12), 6.8 g of the compound represented by Formula (20) was used.

The obtained compound (2F) was subjected to ¹H-NMR measurement and ¹⁹F-NMR measurement, and the structure was identified based on the following results.

¹H-NMR (CD₃COCD₃): δ [ppm]=1.65 to 1.81 (32H), 3.39 to 4.35 (66H)

¹⁹F-NMR (CD₃COCD₃): δ [ppm]=−84.0 to −83.0 (24F), −86.4 (12F), −124.3 (12F), −130.0 to −129.0 (12F)

### Example 29

4.6 g (a molecular weight of 2,567, 1.8 mmol) of the compound represented by Formula (2G) (in Formula (2G), Rf₂2 g is represented by Formula (2GF), and in three Rf₂2g's, n2g indicating an average degree of polymerization is 2.0) was obtained in the same operation as in Example 23 except that, in the second reaction, in place of the compound represented by Formula (12), 6.4 g of the compound represented by Formula (21) was used.

The obtained compound (2G) was subjected to ¹H-NMR measurement and ¹⁹F-NMR measurement, and the structure was identified based on the following results.

¹H-NMR (CD₃COCD₃): δ [ppm]=3.39 to 4.35 (78H)

¹⁹F-NMR (CD₃COCD₃): δ [ppm]=−84.0 to −83.0 (24F), −86.4 (12F), −124.3 (12F), −130.0 to −129.0 (12F)

### Example 30

4.1 g (a number-average molecular weight of 2,257, 1.8 mmol) of the compound represented by Formula (2H) (in Formula (2H), Rf₁2h is represented by Formula (2HF), and in three Rf₁2h's, l2h indicating an average degree of polymerization is 2.4 and m2h indicating an average degree of polymerization is 2.4) was obtained in the same operation as in Example 25 except that, in the second reaction, in place of the compound represented by Formula (15), 2.5 g of the compound represented by Formula (23) was used.

The obtained compound (2H) was subjected to ¹H-NMR measurement and ¹⁹F-NMR measurement, and the structure was identified based on the following results.

¹H-NMR (CD₃COCD₃): δ [ppm]=3.39 to 4.35 (50H)

¹⁹F-NMR (CD₃COCD₃): δ [ppm]=−55.6 to −50.6 (14.4F), −77.7 (6F), −80.3 (6F), −91.0 to −88.5 (28.8F)

### Example 31

4.1 g (a number-average molecular weight of 2,297, 1.8 mmol) of the compound represented by Formula (21) (in Formula (21), Rf₁2i is represented by Formula (2IF), and in three Rf₁2i's, l2i indicating an average degree of polymerization is 3.8 and m2i indicating an average degree of polymerization is 0) was obtained in the same operation as in Example 26 except that, in the second reaction, in place of the compound represented by Formula (16), 2.6 g of the compound represented by Formula (24) was used.

The obtained compound (21) was subjected to ¹H-NMR measurement and ¹⁹F-NMR measurement, and the structure was identified based on the following results.

¹H-NMR (CD₃COCD₃): δ [ppm]=1.65 to 1.81 (2H), 3.39 to 4.35 (62H)

¹⁹F-NMR (CD₃COCD₃): δ [ppm]=−78.6 (6F), −81.3 (6F), −90.0 to −88.5 (45.6F)

### Example 32

4.1 g (a molecular weight of 2,299, 1.8 mmol) of the compound represented by Formula (2J) (in Formula (2J), Rf₂2j is represented by Formula (2JF), and in three Rf₂2j's, n2j indicating an average degree of polymerization is 2.0) was obtained in the same operation as in Example 23 except that, in the second reaction, in place of the compound represented by Formula (12), 2.9 g of the compound represented by Formula (25) was used.

The obtained compound (2J) was subjected to ¹H-NMR measurement and ¹⁹F-NMR measurement, and the structure was identified based on the following results.

¹H-NMR (CD₃COCD₃): δ [ppm]=1.65 to 1.81 (6H), 3.39 to 4.35 (52H)

¹⁹F-NMR (CD₃COCD₃): δ [ppm]=−84.0 to −83.0 (24F), −86.4 (12F), −124.3 (12F), −130.0 to −129.0 (12F)

### Example 33

4.4 g (a number-average molecular weight of 2,437, 1.8 mmol) of the compound represented by Formula (2K) (in Formula (2K), Rf₁2k is represented by Formula (2KF), and in three Rf₁2k's, l2k indicating an average degree of polymerization is 3.8 and m2k indicating an average degree of polymerization is 0) was obtained in the same operation as in Example 26 except that, in the second reaction, in place of the compound represented by Formula (16), 3.6 g of the compound represented by Formula (26) was used.

The obtained compound (2K) was subjected to ¹H-NMR measurement and ¹⁹F-NMR measurement, and the structure was identified based on the following results.

¹H-NMR (CD₃COCD₃): δ [ppm]=1.65 to 1.81 (22H), 3.39 to 4.35 (52H)

¹⁹F-NMR (CD₃COCD₃): δ [ppm]=−78.6 (6F), −81.3 (6F), −90.0 to −88.5 (45.6F)

### Example 34

4.1 g (a molecular weight of 2,271, 1.8 mmol) of the compound represented by Formula (2L) (in Formula (2L), Rf₂21 is represented by Formula (2LF), and in three Rf₂2l's, n21 indicating an average degree of polymerization is 2.0) was obtained in the same operation as in Example 23 except that, in the second reaction, in place of the compound represented by Formula (12), 2.7 g of the compound represented by Formula (27) was used.

The obtained compound (2L) was subjected to ¹H-NMR measurement and ¹⁹F-NMR measurement, and the structure was identified based on the following results.

¹H-NMR (CD₃COCD₃): δ [ppm]=1.65 to 1.81 (4H), 3.39 to 4.35 (50H)

¹⁹F-NMR (CD₃COCD₃): δ [ppm]=−84.0 to −83.0 (24F), −86.4 (12F), −124.3 (12F), −130.0 to −129.0 (12F)

### Example 35

4.4 g (a molecular weight of 2,419, 1.8 mmol) of the compound represented by Formula (2M) (in Formula (2M), Rf₂2m is represented by Formula (2MF), and in three Rf₂2m's, n2m indicating an average degree of polymerization is 2.0) was obtained in the same operation as in Example 23 except that, in the second reaction, in place of the compound represented by Formula (12), 3.7 g of the compound represented by Formula (28) was used.

The obtained compound (2M) was subjected to ¹H-NMR measurement and ¹⁹F-NMR measurement, and the structure was identified based on the following results.

¹H-NMR (CD₃COCD₃): δ [ppm]=1.65 to 1.81 (4H), 3.39 to 4.35 (62H)

¹⁹F-NMR (CD₃COCD₃): δ [ppm]=−84.0 to −83.0 (24F), −86.4 (12F), −124.3 (12F), −130.0 to −129.0 (12F)

### Example 36

4.3 g (a molecular weight of 2,391, 1.8 mmol) of the compound represented by Formula (2N) (in Formula (2N), Rf₂2n is represented by Formula (2NF), and in three Rf₂2n's, n2n indicating an average degree of polymerization is 2.0) was obtained in the same operation as in Example 23 except that, in the second reaction, in place of the compound represented by Formula (12), 3.5 g of the compound represented by Formula (29) was used.

The obtained compound (2N) was subjected to ¹H-NMR measurement and ¹⁹F-NMR measurement, and the structure was identified based on the following results.

¹H-NMR (CD₃COCD₃): δ [ppm]=3.39 to 4.35 (62H)

¹⁹F-NMR (CD₃COCD₃): δ [ppm]=−84.0 to −83.0 (24F), −86.4 (12F), −124.3 (12F), −130.0 to −129.0 (12F)

### Example 37

4.4 g (a molecular weight of 2,419, 1.8 mmol) of the compound represented by Formula (2O) (in Formula (2O), Rf₂2o is represented by Formula (2OF), and in three Rf₂2o's, n2o indicating an average degree of polymerization is 2.0) was obtained in the same operation as in Example 23 except that, in the second reaction, in place of the compound represented by Formula (12), 5.4 g of the compound represented by Formula (31) was used.

The obtained compound (2O) was subjected to ¹H-NMR measurement and ¹⁹F-NMR measurement, and the structure was identified based on the following results.

¹H-NMR (CD₃COCD₃): δ [ppm]=3.39 to 4.35 (66H)

¹⁹F-NMR (CD₃COCD₃): δ [ppm]=−84.0 to −83.0 (24F), −86.4 (12F), −124.3 (12F), −130.0 to −129.0 (12F)

### Example 38

4.5 g (a molecular weight of 2,475, 1.8 mmol) of the compound represented by Formula (2P) (in Formula (2P), Rf₂2p is represented by Formula (2 PF), and in three Rf₂2p's, n2p indicating an average degree of polymerization is 2.0) was obtained in the same operation as in Example 23 except that, in the second reaction, in place of the compound represented by Formula (12), 5.7 g of the compound represented by Formula (33) was used.

The obtained compound (2P) was subjected to ¹H-NMR measurement and ¹⁹F-NMR measurement, and the structure was identified based on the following results.

¹H-NMR (CD₃COCD₃): δ [ppm]=1.65 to 1.81 (10H), 3.39 to 4.35 (64H) ¹⁹F-NMR (CD₃COCD₃): δ [ppm]=−84.0 to −83.0 (24F), −86.4 (12F), −124.3 (12F), −130.0 to −129.0 (12F)

### Example 39

4.6 g (a number-average molecular weight of 2,546, 1.8 mmol) of the compound represented by Formula (2Q) (in Formula (2Q), Rf₁2q is represented by Formula (2QF), and in three Rf₁2q;s, l2q indicating an average degree of polymerization is 2.4 and m2q indicating an average degree of polymerization is 2.4) was obtained in the same operation as in Example 25 except that, in the second reaction, in place of the compound represented by Formula (15), 6.1 g of the compound represented by Formula (35) was used.

The obtained compound (2Q) was subjected to ¹H-NMR measurement and ¹⁹F-NMR measurement, and the structure was identified based on the following results.

¹H-NMR (CD₃COCD₃): δ [ppm]=1.65 to 1.81 (18H), 3.39 to 4.35 (64H)

¹⁹F-NMR (CD₃COCD₃): δ [ppm]=−55.6 to −50.6 (14.4F), −77.7 (6F), −80.3 (6F), −91.0 to −88.5 (28.8F)

### Example 40

4.8 g (a number-average molecular weight of 2,670, 1.8 mmol) of the compound represented by Formula (2R) (in Formula (2R), Rf₁2r is represented by Formula (2RF), and in three Rf₁2r's, l2r indicating an average degree of polymerization is 3.8 and m2r indicating an average degree of polymerization is 0) was obtained in the same operation as in Example 26 except that, in the second reaction, in place of the compound represented by Formula (16), 6.8 g of the compound represented by Formula (37) was used.

The obtained compound (2R) was subjected to ¹H-NMR measurement and ¹⁹F-NMR measurement, and the structure was identified based on the following results.

¹H-NMR (CD₃COCD₃): δ [ppm]=1.65 to 1.81 (32H), 3.39 to 4.35 (66H)

¹⁹F-NMR (CD₃COCD₃): δ [ppm]=−78.6 (6F), −81.3 (6F), −90.0 to −88.5 (45.6F)

### Example 41

4.8 g (a number-average molecular weight of 2,686, 1.8 mmol) of the compound represented by Formula (2S) (in Formula (2S), Rf₁2s is represented by Formula (2SF), and in three Rf₁2s's, l2s indicating an average degree of polymerization is 2.4 and m2s indicating an average degree of polymerization is 2.4) was obtained in the same operation as in Example 25 except that, in the second reaction, in place of the compound represented by Formula (15), 7.0 g of the compound represented by Formula (39) was used.

The obtained compound (2S) was subjected to ¹H-NMR measurement and ¹⁹F-NMR measurement, and the structure was identified based on the following results.

¹H-NMR (CD₃COCD₃): δ [ppm]=1.65 to 1.81 (36H), 3.39 to 4.35 (66H) ¹⁹F-NMR (CD₃COCD₃): δ [ppm]=−55.6 to −50.6 (14.4F), −77.7 (6F), −80.3 (6F), −91.0 to −88.5 (28.8F)

### Example 42

5.1 g (a number-average molecular weight of 2,810, 1.8 mmol) of the compound represented by Formula (2T) (in Formula (2T), Rf₁2t is represented by Formula (2TF), and in three Rf₁2t's, l2t indicating an average degree of polymerization is 3.8 and m2t indicating an average degree of polymerization is 0) was obtained in the same operation as in Example 26 except that, in the second reaction, in place of the compound represented by Formula (16), 7.7 g of the compound represented by Formula (41) was used.

The obtained compound (2T) was subjected to ¹H-NMR measurement and ¹⁹F-NMR measurement, and the structure was identified based on the following results.

¹H-NMR (CD₃COCD₃): δ [ppm]=1.65 to 1.81 (52H), 3.39 to 4.35 (66H)

¹⁹F-NMR (CD₃COCD₃): > [ppm]=−78.6 (6F), −81.3 (6F), −90.0 to −88.5 (45.6F)

### Example 43

4.7 g (a molecular weight of 2,595, 1.8 mmol) of the compound represented by Formula (2U) (in Formula (2U), Rf₂2u is represented by Formula (2UF), and in three Rf₂2u's, n2u indicating an average degree of polymerization is 2.0) was obtained in the same operation as in Example 23 except that, in the second reaction, in place of the compound represented by Formula (12), 6.5 g of the compound represented by Formula (43) was used.

The obtained compound (2U) was subjected to ¹H-NMR measurement and ¹⁹F-NMR measurement, and the structure was identified based on the following results.

¹H-NMR (CD₃COCD₃): δ [ppm]=3.39 to 4.35 (82H)

¹⁹F-NMR (CD₃COCD₃): δ [ppm]=−84.0 to −83.0 (24F), −86.4 (12F), −124.3 (12F), −130.0 to −129.0 (12F)

### Example 44

4.2 g (a molecular weight of 2,331, 1.8 mmol) of the compound represented by Formula (2V) (in Formula (2V), Rf₂2v is represented by Formula (2VF), and in three Rf₂2v's, n2v indicating an average degree of polymerization is 2.0) was obtained in the same operation as in Example 23 except that, in the second reaction, in place of the compound represented by Formula (12), 5.9 g of the compound represented by Formula (45) was used.

The obtained compound (2V) was subjected to ¹H-NMR measurement and ¹⁹F-NMR measurement, and the structure was identified based on the following results.

¹H-NMR (CD₃COCD₃): δ [ppm]=3.39 to 4.35 (58H)

¹⁹F-NMR (CD₃COCD₃): δ [ppm]=−84.0 to −83.0 (24F), −86.4 (12F), −124.3 (12F), −130.0 to −129.0 (12F)

The values of z when the compounds (1A) to (1V), and (2A) to (2V) of Examples 1 to 44 thus obtained were applied to Formula (1), and the structures of R¹, [A], [B], R², R³, [C], [D], and R⁴ are shown in Table 5 to Table 9.

TABLE 5

| | | | | | | | R³ = | | |
|---|---|---|---|---|---|---|---|---|---|
| | Compound | z | R¹ = R⁴ | [B] | [A] | R² | (Formula (5)) | [C] | [D] |
| Example 1 | (1A) | 1 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 1 | b = 0 | a = 1 | n = 3.8 | | d = 1 | e = 0 |
| | | | X¹ = X² = | | | | | | |
| | | | Formula (7) | | | | | | |
| | | | s = 2, t = 1 | | | | | | |
| Example 2 | (1B) | 1 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 1 | b = 0 | a = 1 | n = 3.8 | | d = 1 | e = 0 |
| | | | X¹, X² = | | | | | | |
| | | | Formula (7) | | | | | | |
| | | | X¹: s = 2, t = 1 | | | | | | |
| | | | X²: s = 3, t = 1 | | | | | | |
| Example 3 | (1C) | 1 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 1 | b = 0 | a = 1 | l = 4.0 | | d = 1 | e = 0 |
| | | | X¹ = X² = | | | m = 4.0 | | | |
| | | | Formula (7) | | | | | | |
| | | | s = 3, t = 1 | | | | | | |
| Example 4 | (1D) | 1 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 1 | b = 0 | a = 1 | l = 6.3 | | d = 1 | e = 0 |
| | | | X¹ = X² = | | | m = 0 | | | |
| | | | Formula (7) | | | | | | |
| | | | s = 4, t = 1 | | | | | | |
| Example 5 | (1E) | 1 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 2 | b = 1 | a = 0 | n = 3.8 | | d = 0 | e = 1 |
| | | | X¹ = X² = | c = 2 | | | | | f = 2 |
| | | | Formula (7) | | | | | | |
| | | | s = 2, t = 1 | | | | | | |
| Example 6 | (1F) | 1 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 5 | b = 1 | a = 0 | n = 3.8 | | d = 0 | e = 1 |
| | | | X¹ = X² = | c = 5 | | | | | f = 5 |
| | | | Formula (7) | | | | | | |
| | | | s = 2, t = 1 | | | | | | |
| Example 7 | (1G) | 1 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 1 | b = 0 | a = 2 | n = 3.8 | | d = 2 | e = 0 |
| | | | X¹ = X² = | | | | | | |
| | | | Formula (7) | | | | | | |
| | | | s = 2, t = 1 | | | | | | |
| Example 8 | (1H) | 1 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 0, i = 1, | b = 0 | a = 1 | l = 4.0 | | d = 1 | e = 0 |
| | | | j = 1 | | | m = 4.0 | | | |
| | | | X³ = X⁴ = H | | | | | | |
| Example 9 | (1I) | 1 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 1, i = 1, | b = 0 | a = 1 | l = 6.3 | | d = 1 | e = 0 |
| | | | j = 1 | | | m = 0 | | | |
| | | | X³ = X⁴ = H | | | | | | |
| Example 10 | (1J) | 1 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 2, i = 1, | b = 0 | a = 1 | n = 3.8 | | d = 1 | e = 0 |
| | | | j = 1 | | | | | | |
| | | | X³ = X⁴ = H | | | | | | |

TABLE 6

| | | | | | | | R³ = | | |
|---|---|---|---|---|---|---|---|---|---|
| | Compound | z | R¹ = R⁴ | [B] | [A] | R² | (Formula (5)) | [C] | [D] |
| Example 11 | (1K) | 1 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 6, i = 1, | b = 0 | a = 1 | l = 6.3 | | d = 1 | e = 0 |
| | | | j = 1 | | | m = 0 | | | |
| | | | X³ = X⁴ = H | | | | | | |
| Example 12 | (1L) | 1 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 1 | a = 0 | n = 3.8 | | d = 0 | e = 1 |
| | | | j = 1 | c = 2 | | | | | f = 2 |
| | | | X³ = X⁴ = H | | | | | | |
| Example 13 | (1M) | 1 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 1 | a = 1 | n = 3.8 | | d = 1 | e = 1 |
| | | | j = 1 | c = 2 | | | | | f = 2 |
| | | | X³ = X⁴ = H | | | | | | |
| Example 14 | (1N) | 1 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 2 | n = 3.8 | | d = 2 | e = 0 |
| | | | j = 1 | | | | | | |
| | | | X³ = X⁴ = H | | | | | | |
| Example 15 | (1O) | 1 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 1 | n = 3.8 | | d = 1 | e = 0 |
| | | | j = 1 | | | | | | |
| | | | X³ = X⁴ = | | | | | | |
| | | | Formula (7) | | | | | | |
| | | | s = 2, t = 1 | | | | | | |
| Example 16 | (1P) | 1 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 1 | n = 3.8 | | d = 1 | e = 0 |
| | | | j = 1 | | | | | | |
| | | | X³ = X⁴ = | | | | | | |
| | | | Formula (7) | | | | | | |
| | | | s = 3, t = 1 | | | | | | |
| Example 17 | (1Q) | 1 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 0, i = 1, | b = 0 | a = 1 | l = 4.0 | | d = 1 | e = 0 |
| | | | j = 1 | | | m = 4.0 | | | |
| | | | X³ = X⁴ = | | | | | | |
| | | | Formula (7) | | | | | | |
| | | | s = 4, t = 1 | | | | | | |
| Example 18 | (1R) | 1 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 0, i = 1, | b = 0 | a = 1 | l = 6.3 | | d = 1 | e = 0 |
| | | | j = 1 | | | m = 0 | | | |
| | | | X³ = X⁴ = | | | | | | |
| | | | Formula (7) | | | | | | |
| | | | s = 6, t = 1 | | | | | | |
| Example 19 | (1S) | 1 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 2 | b = 0 | a = 1 | l = 4.0 | | d = 1 | e = 0 |
| | | | X¹ = X² = | | | m = 4.0 | | | |
| | | | Formula (7) | | | | | | |
| | | | s = 6, t = 1 | | | | | | |
| Example 20 | (1T) | 1 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 6 | b = 0 | a = 1 | l = 6.3 | | d = 1 | e = 0 |
| | | | X¹ = X² = | | | m = 0 | | | |
| | | | Formula (7) | | | | | | |
| | | | s = 6, t = 1 | | | | | | |

TABLE 7

| | | | | | | | R³ = | | |
|---|---|---|---|---|---|---|---|---|---|
| | Compound | z | R¹ = R⁴ | [B] | [A] | R² | (Formula (5)) | [C] | [D] |
| Example 21 | (1U) | 1 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 1 | n = 3.8 | | d = 1 | e = 0 |
| | | | j = 1 | | | | | | |
| | | | X³ = X⁴ = | | | | | | |
| | | | Formula (7) | | | | | | |
| | | | s = 2, t = 2 | | | | | | |
| Example 22 | (1V) | 1 | Formula | Formula | Formula | Formula | y1 = 1 | Formula | Formula |
| | | | (6-3) | (2-2) | (2-1) | (10-2) | y2 = 1 | (3-1) | (3-2) |
| | | | k = 1, p = 1 | b = 0 | a = 1 | n = 3.8 | | d = 1 | e = 0 |
| | | | q = 1, r = 1 | | | | | | |
| | | | X⁵ = X⁶ = X⁷ = H | | | | | | |
| Example 23 | (2A) | 2 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 1 | b = 0 | a = 1 | n = 2.0 | | d = 1 | e = 0 |
| | | | X¹ = X² = | | | | | | |
| | | | Formula (7) | | | | | | |
| | | | s = 2, t = 1 | | | | | | |
| Example 24 | (2B) | 2 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 1 | b = 0 | a = 1 | n = 2.0 | | d = 1 | e = 0 |
| | | | X¹, X² = | | | | | | |
| | | | Formula (7) | | | | | | |
| | | | X¹: s = 2, | | | | | | |
| | | | t = 1 | | | | | | |
| | | | X²: s = 3, | | | | | | |
| | | | t = 1 | | | | | | |
| Example 25 | (2C) | 2 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 1 | b = 0 | a = 1 | l = 2.4 | | d = 1 | e = 0 |
| | | | X¹ = X² = | | | m = 2.4 | | | |
| | | | Formula (7) | | | | | | |
| | | | s = 3, t = 1 | | | | | | |
| Example 26 | (2D) | 2 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 1 | b = 0 | a = 1 | l = 3.8 | | d = 1 | e = 0 |
| | | | X¹ = X² = | | | m = 0 | | | |
| | | | Formula (7) | | | | | | |
| | | | s = 4, t = 1 | | | | | | |
| Example 27 | (2E) | 2 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 2 | b = 1 | a = 0 | n = 2.0 | | d = 0 | e = 1 |
| | | | X¹ = X² = | c = 2 | | | | | f = 2 |
| | | | Formula (7) | | | | | | |
| | | | s = 2, t = 1 | | | | | | |
| Example 28 | (2F) | 2 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 5 | b = 1 | a = 0 | n = 2.0 | | d = 0 | e = 1 |
| | | | X¹ = X² = | c = 5 | | | | | f = 5 |
| | | | Formula (7) | | | | | | |
| | | | s = 2, t = 1 | | | | | | |
| Example 29 | (2G) | 2 | Formula | Formula | Formula | Formula | y1 = 1 | Formula | Formula |
| | | | (6-1) | (2-2) | (2-1) | (10-2) | y2 = 1 | (3-1) | (3-2) |
| | | | g = 1 | b = 0 | a = 2 | n = 2.0 | | d = 2 | e = 0 |
| | | | X¹ = X² = | | | | | | |
| | | | Formula (7) | | | | | | |
| | | | s = 2, t = 1 | | | | | | |
| Example 30 | (2H) | 2 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 0, i = 1, | b = 0 | a = 1 | l = 2.4 | | d = 1 | e = 0 |
| | | | j = 1 | | | m = 2.4 | | | |
| | | | X³ = X⁴ = H | | | | | | |

TABLE 8

| | | | | | | | R³ = | | |
|---|---|---|---|---|---|---|---|---|---|
| | Compound | z | R¹ = R⁴ | [B] | [A] | R² | (Formula (5)) | [C] | [D] |
| Example 31 | (2I) | 2 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 1, i = 1, | b = 0 | a = 1 | l = 3.8 | | d = 1 | e = 0 |
| | | | j = 1 | | | m = 0 | | | |
| | | | X³ = X⁴ = H | | | | | | |
| Example 32 | (2J) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 2, i = 1, | b = 0 | a = 1 | n = 2.0 | | d = 1 | e = 0 |
| | | | j = 1 | | | | | | |
| | | | X³ = X⁴ = H | | | | | | |
| Example 33 | (2K) | 2 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 6, i = 1, | b = 0 | a = 1 | l = 3.8 | | d = 1 | e = 0 |
| | | | j = 1 | | | m = 0 | | | |
| | | | X³ = X⁴ = H | | | | | | |
| Example 34 | (2L) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 1 | a = 0 | n = 2.0 | | d = 0 | e = 1 |
| | | | j = 1 | c = 2 | | | | | f = 2 |
| | | | X³ = X⁴ = H | | | | | | |
| Example 35 | (2M) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 1 | a = 1 | n = 2.0 | | d = 1 | e = 1 |
| | | | i = 1 | c = 2 | | | | | f = 2 |
| | | | X³ = X⁴ = H | | | | | | |
| Example 36 | (2N) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 2 | n = 2.0 | | d = 2 | e = 0 |
| | | | j = 1 | | | | | | |
| | | | X³ = X⁴ = H | | | | | | |
| Example 37 | (2O) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 1 | n = 2.0 | | d = 1 | e = 0 |
| | | | j = 1 | | | | | | |
| | | | X³ = X⁴ = | | | | | | |
| | | | Formula (7) | | | | | | |
| | | | s = 2, t = 1 | | | | | | |
| Example 38 | (2P) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 1 | n = 2.0 | | d = 1 | e = 0 |
| | | | j = 1 | | | | | | |
| | | | X³ = X⁴ = | | | | | | |
| | | | Formula (7) | | | | | | |
| | | | s = 3, t = 1 | | | | | | |
| Example 39 | (2Q) | 2 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 0, i = 1, | b = 0 | a = 1 | l = 2.4 | | d = 1 | e = 0 |
| | | | j = 1 | | | m = 2.4 | | | |
| | | | X³ = X⁴ = | | | | | | |
| | | | Formula (7) | | | | | | |
| | | | s = 4, t = 1 | | | | | | |
| Example 40 | (2R) | 2 | Formula | Formula | Formula | Formula | y1 = 1 | Formula | Formula |
| | | | (6-2) | (2-2) | (2-1) | (10-1) | y2 = 1 | (3-1) | (3-2) |
| | | | h = 0, i = 1, | b = 0 | a = 1 | l = 3.8 | | d = 1 | e = 0 |
| | | | j = 1 | | | m = 0 | | | |
| | | | X³ = X⁴ = | | | | | | |
| | | | Formula (7) | | | | | | |
| | | | s = 6, t = 1 | | | | | | |

TABLE 9

| | | | | | | | R³ = | | |
|---|---|---|---|---|---|---|---|---|---|
| | Compound | z | R¹ = R⁴ | [B] | [A] | R² | (Formula (5)) | [C] | [D] |
| Example 41 | (2S) | 2 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 2 | b = 0 | a = 1 | l = 2.4 | | d = 1 | e = 0 |
| | | | X¹ = X² = | | | m = 2.4 | | | |
| | | | Formula (7) | | | | | | |
| | | | s = 6, t = 1 | | | | | | |
| Example 42 | (2T) | 2 | Formula | Formula | Formula | Formula | y1 = 1 | Formula | Formula |
| | | | (6-1) | (2-2) | (2-1) | (10-1) | y2 = 1 | (3-1) | (3-2) |
| | | | g = 6 | b = 0 | a = 1 | l = 3.8 | | d = 1 | e = 0 |
| | | | X¹ = X² = | | | m = 0 | | | |
| | | | Formula (7) | | | | | | |
| | | | s = 6, t = 1 | | | | | | |
| Example 43 | (2U) | 2 | Formula | Formula | Formula | Formula | y1 = 1 | Formula | Formula |
| | | | (6-2) | (2-2) | (2-1) | (10-2) | y2 = 1 | (3-1) | (3-2) |
| | | | h = 0, i = 1, | b = 0 | a = 1 | n = 2.0 | | d = 1 | e = 0 |
| | | | j = 1 | | | | | | |
| | | | X³ = X⁴ = | | | | | | |
| | | | Formula (7) | | | | | | |
| | | | s = 2, t = 2 | | | | | | |
| Example 44 | (2V) | 2 | Formula | Formula | Formula | Formula | y1 = 1 | Formula | Formula |
| | | | (6-3) | (2-2) | (2-1) | (10-2) | y2 = 1 | (3-1) | (3-2) |
| | | | k = 1, p = 1, | b = 0 | a = 1 | n = 2.0 | | d = 1 | e = 0 |
| | | | q = 1, r = 1 | | | | | | |
| | | | X⁵ = X⁶ = X⁷ = H | | | | | | |

### Comparative Example 1

A compound represented by the following Formula (3A) was synthesized by the method described in Patent Document 1.

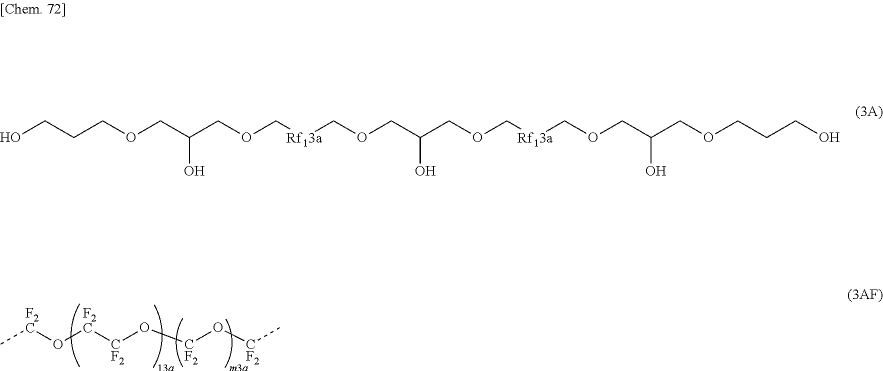

(in Formula (3A), Rf₁₃a is the PFPE chain represented by Formula (3AF), and in two Rf₁₃a's, 13a indicating an average degree of polymerization represents 4.0, and m3a indicating an average degree of polymerization represents 4.0).

### Comparative Example 2

A compound represented by the following Formula (3B) was synthesized by the method described in Patent Document 2.

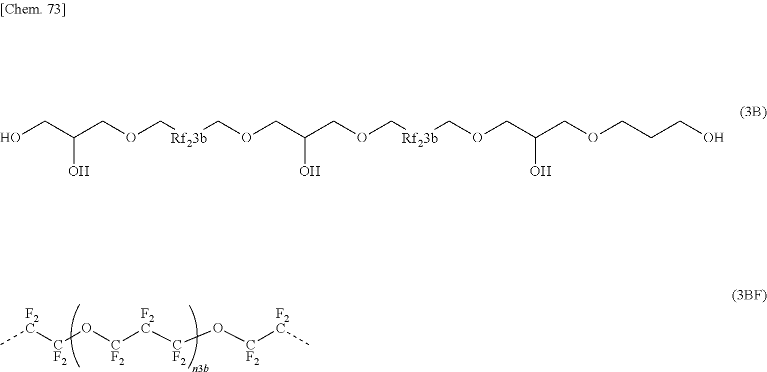

(in Formula (3B), Rf₂3b is the PFPE chain represented by Formula (3BF), and in two Rf₂3b's, n3b indicating an average degree of polymerization represents 3.8).

### Comparative Example 3

A compound represented by the following Formula (3C) was synthesized by the method described in Patent Document 4.

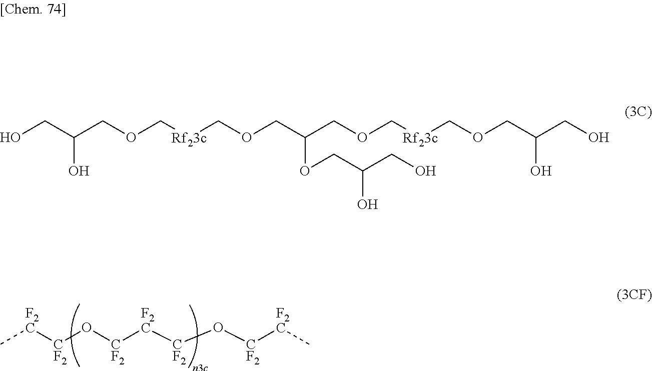

(in Formula (3C), Rf₂3c is the PFPE chain represented by Formula (3CP), and in two Rf₂3c's, n3c indicating an average degree of polymerization represents 3.8).

### Comparative Example 4

A compound represented by the following Formula (3D) was synthesized by the method described in Patent Document 4.

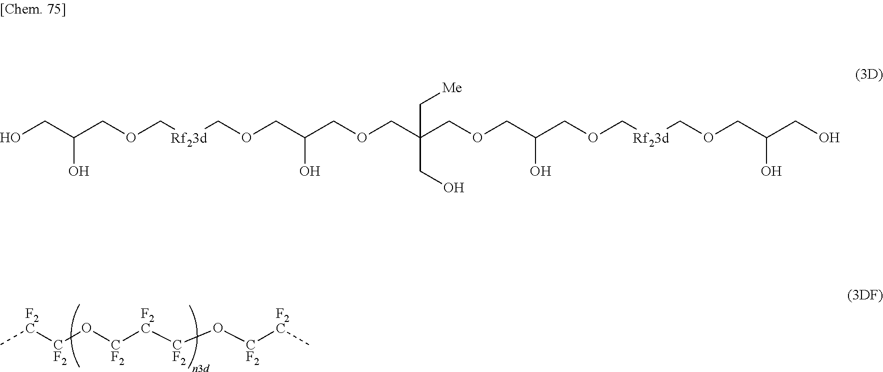

(in Formula (3D), Rf₂3d is the PFPE chain represented by Formula (3DF), Me represents a methyl group, and in two Rf₂3d's, n3d indicating an average degree of polymerization represents 3.8).

### Comparative Example 5

A compound represented by the following Formula (3E) was synthesized by the method described in Patent Document 4.

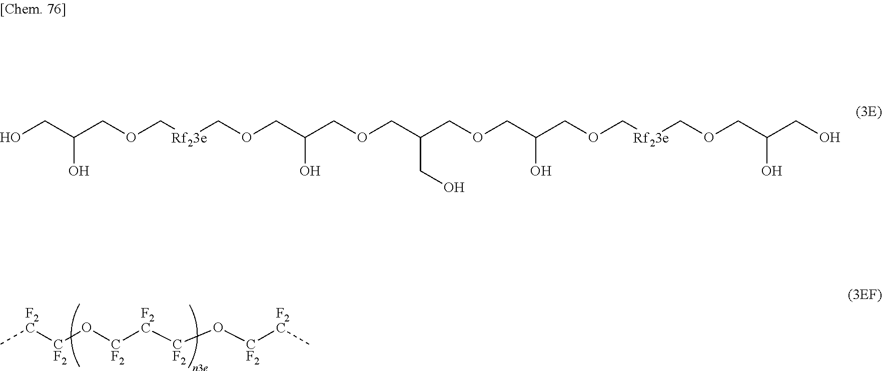

(in Formula (3E), Rf₂3e is the PFPF chain represented by Formula (3EF), and in two Rf₂3e's, n3e indicating an average degree of polymerization represents 3.8).

### Comparative Example 6

A compound represented by the following Formula (3F) was synthesized by the method described in Patent Document 3.

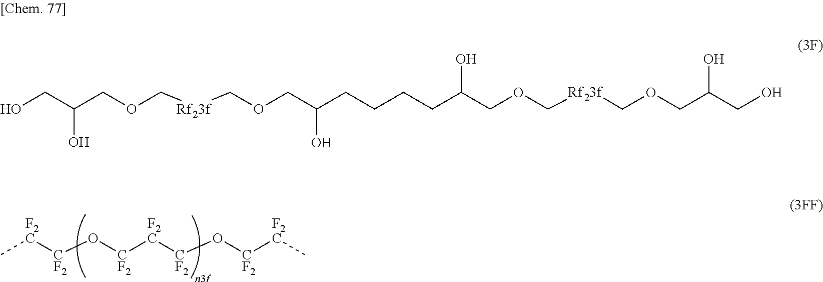

(in Formula (3F), Rf₂3f is the PFPE chain represented by Formula (3FF), and in two Rf₂3f's, n3f indicating an average degree of polymerization represents 3.8).

### Comparative Example 7

A compound represented by the following Formula (3G) was synthesized by the method described in Patent Document 6.

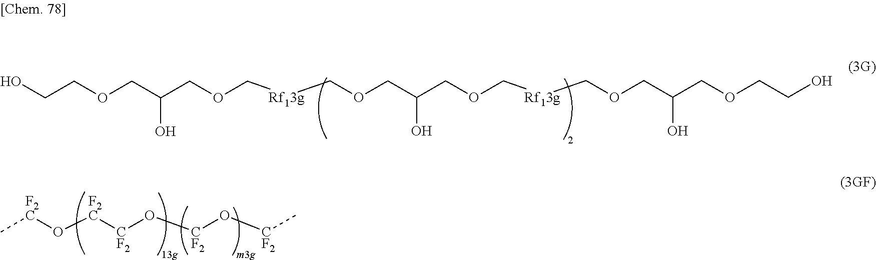

(in Formula (3G), Rf₁₃g is the PFPF chain represented by Formula (3GF), in the center Rf₁₃g among three Rf₁₃g's, 13g indicating an average degree of polymerization represents 3.8 and m3g indicating an average degree of polymerization represents 0, and in two Rf₁₃g's on the terminal side, 13g indicating an average degree of polymerization represents 2.4 and m3g indicating an average degree of polymerization represents 2.4).

### Comparative Example 8

A compound represented by the following Formula (3H) was synthesized by the method described in Patent Document 7.

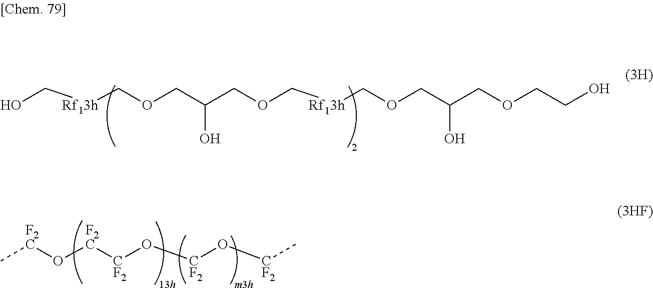

(in Formula (3H), Rf₁₃h is the PFPF chain represented by Formula (3HF), and in three Rf₁₃h's, 13h indicating an average degree of polymerization represents 3.8 and m3h indicating an average degree of polymerization represents 0).

### Comparative Example 9

A compound represented by the following Formula (31) was synthesized by the method described in Patent Document 5.

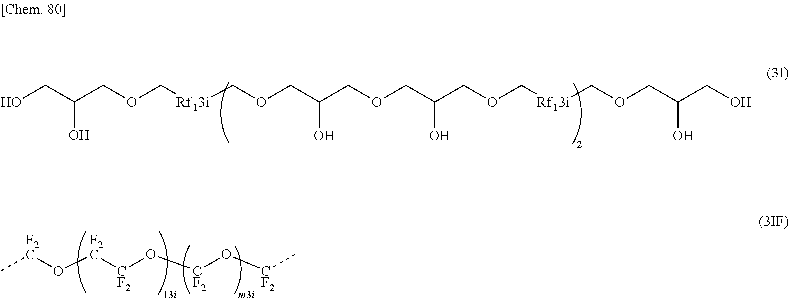

(in Formula (31), Rf₁₃i is the PFPF chain represented by Formula (3IF), and in three Rf₁₃i's, 13i indicating an average degree of polymerization represents 3.8, and m3i indicating an average degree of polymerization represents 0).

### Comparative Example 10

A compound represented by the following Formula (3J) was synthesized by the method described in Patent Document 8.

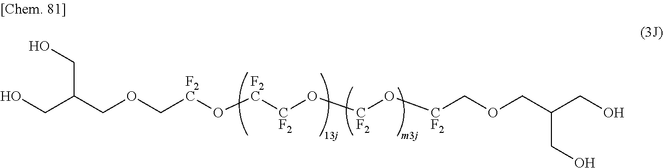

(in Formula (3J), 13j indicating an average degree of polymerization represents 4.5, and m3j indicating an average degree of polymerization represents 4.5).

### Comparative Example 11

A compound represented by the following Formula (3K) was synthesized by the method described in Patent Document 8.

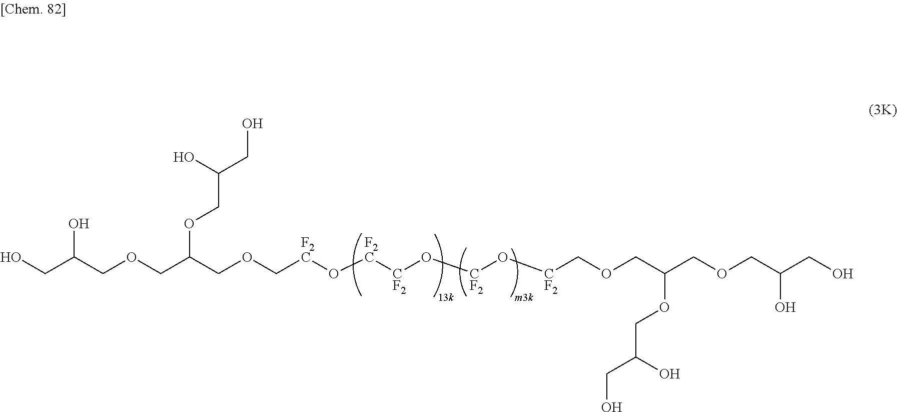

(in Formula (3K), 13k indicating an average degree of polymerization represents 4.5, and m3k indicating an average degree of polymerization represents 4.5).

The number-average molecular weight (Mn) of the compounds of Examples 1 to 44 and Comparative Examples 1 to 11 thus obtained was measured by the method. The results are shown in Table 10 to Table 12.

Next, by the following method, lubricating layer forming solutions were prepared using the compounds obtained in Examples 1 to 44 and Comparative Examples 1 to 11. Then, lubricating layers of magnetic recording media were formed using the obtained lubricating layer forming solution by the following method, and magnetic recording media of Examples 1 to 44 and Comparative Examples 1 to 11 were obtained.

### [Lubricating Layer Forming Solution]

The compounds obtained in Examples 1 to 44 and Comparative Examples 1 to 11 were each dissolved in Vertel® XF (product name, commercially available from Du Pont-Mitsui Fluorochemicals Co., Ltd.) as a fluorine-based solvent and diluted with Vertel XF so that the film thickness when applied onto the protective layer was 9.4 Å to 9.7 Å, and thereby a lubricating layer forming solution having a compound concentration of 0.001 mass % to 0.01 mass % was obtained.

### “Magnetic Recording Medium”

A magnetic recording medium in which an adhesive layer, a soft magnetic layer, a first base layer, a second base layer, a magnetic layer and a protective layer were sequentially provided on a substrate with a diameter of 65 mm was prepared. The protective layer was made of carbon.

The lubricating layer forming solutions of Examples 1 to 44 and Comparative Examples 1 to 11 were applied onto the protective layer of the magnetic recording medium in which respective layers up to the protective layer were formed by a dipping method. Here, the dipping method was performed under conditions of an immersion speed of 10 mm/see, an immersion time of 30 sec, and a lifting speed of 1.2 mm/sec.

Then, the magnetic recording medium to which the lubricating layer forming solution was applied was put into a thermostatic chamber and subjected to a heat treatment at 120° C. for 10 minutes in order to remove the solvent in the lubricating layer forming solution and improve the adhesion between the protective layer and the lubricating layer, and thus a lubricating layer was formed on the protective layer to obtain a magnetic recording medium.

### (Measurement of Film Thickness)

Regarding the lubricating layers of the magnetic recording media of Examples 1 to 44 and Comparative Examples 1 to 11 obtained in this manner, the peak height in C—F vibration expansion and contraction was measured using a Fourier transform infrared spectrophotometer (FT-IR, product name: Nicolet iS50, commercially available from Thermo Fisher Scientific). Next, using a correlation formula determined by a method to be described below, the film thickness of the lubricating layer was calculated from the measured value of the peak height in C—F vibration expansion and contraction of the lubricating layer.

### (Method of Calculating Correlation Equation)

A disk in which an adhesive layer, a soft magnetic layer, a first base layer, a second base layer, a magnetic layer and a protective layer were sequentially provided on a substrate with a diameter of 65 mm was prepared. A lubricating layer was formed on the protective layer of the disk with a film thickness of 6 to 20 Å (in increments of 2 Å). Then, for each disk on which the lubricating layer was formed, the film thickness increment from the surface of the disk on which no lubricating layer was formed was measured using an ellipsometer, and used as the film thickness of the lubricating layer. In addition, for each disk on which the lubricating layer was formed, the peak height in the C—F vibration expansion and contraction was measured using FT-IR.

Then, a correlation formula between the peak height obtained by FT-IR and the film thickness of the lubricating layer obtained using an ellipsometer was obtained.

Next, the magnetic recording media of Examples 1 to 44 and Comparative Examples 1 to 11 were subjected to the following pickup characteristic test, spin-off characteristic test, and corrosion resistance test, and evaluated. The results are shown in Table 10 to Table 12.

### (Pickup Characteristic Test)

A magnetic recording medium and a magnetic head were mounted on a spin stand, rotation was performed under a reduce pressure at room temperature (about 250 torr), and the magnetic head was floated from a fixed point for 10 minutes. Then, the surface of the magnetic head facing the magnetic recording medium was analyzed using an Electron Spectroscopy for Chemical Analysis (ESCA) analysis device. The intensity of the fluorine-derived peak (signal intensity (a.u.)) obtained by analysis using the ESCA analysis device indicated the amount of the lubricant adhered to the magnetic head.

Using the obtained signal intensity, based on the following evaluation criteria, pickup characteristics were evaluated.

### (Evaluation Criteria for Pickup Characteristic)

A (excellent): the signal intensity was 160 or less (very small adhesion amount)
B (good): the signal intensity was 161 to 30 0 (small adhesion amount)
C (acceptable): the signal intensity was 301 to 1,000 (large adhesion amount)
D (poor): the signal intensity was 1,001 or more (very large adhesion amount)

### (Spin-Off Characteristic Test)

A magnetic recording medium was mounted on a spin stand, and rotated under an environment at 80° C. and at a rotational speed of 10,000 rpm for 72 hours. Before and after this operation, the film thickness of the lubricating layer at a position with a radius of 20 mm from the center of the magnetic recording medium was measured using FT-IR by the method, and the film thickness decrement of the lubricating layer before and after the test was calculated. Using the calculated film thickness decrement, based on the following evaluation criteria, spin-off characteristics were evaluated.

### (Evaluation Criteria for Spin-Off Characteristic)

A (excellent): the film thickness decrement was 2% or less
B (good): the film thickness decrement was more than 2% and 3% or less
C (acceptable): the film thickness decrement was more than 3% and 8% or less
D (poor): the film thickness decrement was more than 8%

### (Corrosion Resistance Test)

The magnetic recording medium was exposed under conditions of a temperature of 85° C. and a relative humidity of 90% for 48 hours. Then, the number of corrosion spots with a diameter of 5 μm or more generated on the surface of the magnetic recording medium was counted using an optical surface analysis device (Candela 7140 commercially available from KLA-Tencor), and evaluated based on the following evaluation criteria.

### (Evaluation Criteria for Corrosion Resistance Test)

A (excellent): less than 150 locations
B (good): 150 locations or more and less than 200 locations
C (acceptable): 200 locations or more and less than 400 locations
D (poor): 400 locations or more

TABLE 10

| | | Number- | | | | |
|---|---|---|---|---|---|---|
| | | average | | | | |
| | | molecular | Film | Pickup | Spin-off | Corrosion |
| | | weight | thickness | characteristic | characteristic | resistance |
| | Compound | (Mn) | (Å) | test | test | test |
| Example 1 | (1A) | 2349 | 9.5 | A | A | A |
| Example 2 | (1B) | 2377 | 9.5 | A | A | A |
| Example 3 | (1C) | 2399 | 9.4 | A | A | A |
| Example 4 | (1D) | 2460 | 9.5 | A | A | A |
| Example 5 | (1E) | 2405 | 9.4 | A | A | A |
| Example 6 | (1F) | 2573 | 9.4 | B | B | A |
| Example 7 | (1G) | 2497 | 9.6 | A | A | B |
| Example 8 | (1H) | 2167 | 9.4 | A | A | A |
| Example 9 | (1I) | 2200 | 9.4 | A | A | A |
| Example 10 | (1J) | 2228 | 9.5 | A | A | B |
| Example 11 | (1K) | 2340 | 9.4 | B | B | A |
| Example 12 | (1L) | 2200 | 9.6 | A | A | A |
| Example 13 | (1M) | 2349 | 9.5 | A | A | A |
| Example 14 | (1N) | 2321 | 9.5 | A | A | A |
| Example 15 | (1O) | 2349 | 9.5 | A | A | A |
| Example 16 | (1P) | 2405 | 9.6 | A | A | A |
| Example 17 | (1Q) | 2455 | 9.6 | A | A | A |
| Example 18 | (1R) | 2573 | 9.5 | A | A | A |
| Example 19 | (1S) | 2595 | 9.5 | A | A | A |
| Example 20 | (1T) | 2713 | 9.4 | A | A | A |
| Example 21 | (1U) | 2525 | 9.6 | A | A | A |
| Example 22 | (1V) | 2260 | 9.5 | A | A | B |
| Example 23 | (2A) | 2419 | 9.5 | A | A | A |
| Example 24 | (2B) | 2447 | 9.5 | A | A | A |
| Example 25 | (2C) | 2490 | 9.4 | A | A | A |
| Example 26 | (2D) | 2557 | 9.5 | A | A | A |
| Example 27 | (2E) | 2475 | 9.5 | A | A | A |
| Example 28 | (2F) | 2644 | 9.5 | B | B | A |
| Example 29 | (2G) | 2567 | 9.6 | A | A | A |
| Example 30 | (2H) | 2257 | 9.6 | A | A | A |

TABLE 11

| | | Number- | | | | |
|---|---|---|---|---|---|---|
| | | average | | | | |
| | | molecular | Film | Pickup | Spin-off | Corrosion |
| | | weight | thickness | characteristic | characteristic | resistance |
| | Compound | (Mn) | (Å) | test | test | test |
| Example 31 | (2I) | 2297 | 9.5 | A | A | A |
| Example 32 | (2J) | 2299 | 9.5 | A | A | A |
| Example 33 | (2K) | 2437 | 9.4 | B | B | A |
| Example 34 | (2L) | 2271 | 9.6 | A | A | A |
| Example 35 | (2M) | 2419 | 9.5 | A | A | A |
| Example 36 | (2N) | 2391 | 9.5 | A | A | A |
| Example 37 | (2O) | 2419 | 9.5 | A | A | A |
| Example 38 | (2P) | 2475 | 9.4 | A | A | A |
| Example 39 | (2Q) | 2546 | 9.7 | A | A | A |
| Example 40 | (2R) | 2670 | 9.5 | A | A | A |
| Example 41 | (2S) | 2686 | 9.5 | A | A | A |
| Example 42 | (2T) | 2810 | 9.6 | A | A | A |
| Example 43 | (2U) | 2595 | 9.6 | A | A | A |
| Example 44 | (2V) | 2331 | 9.5 | A | A | A |

TABLE 12

| | | Number- | | | | |
|---|---|---|---|---|---|---|
| | | average | | | | |
| | | molecular | Film | Pickup | Spin-off | Corrosion |
| | | weight | thickness | characteristic | characteristic | resistance |
| | Compound | (Mn) | (Å) | test | test | test |
| Comparative | (3A) | 2133 | 9.4 | C | C | A |
| Example 1 | | | | | | |
| Comparative | (3B) | 2022 | 9.6 | D | D | C |
| Example 2 | | | | | | |
| Comparative | (3C) | 2096 | 9.5 | D | D | C |
| Example 3 | | | | | | |
| Comparative | (3D) | 2064 | 9.5 | D | D | C |
| Example 4 | | | | | | |
| Comparative | (3E) | 2184 | 9.5 | D | D | C |
| Example 5 | | | | | | |
| Comparative | (3F) | 2108 | 9.6 | D | D | C |
| Example 6 | | | | | | |
| Comparative | (3G) | 2197 | 9.6 | C | C | A |
| Example 7 | | | | | | |
| Comparative | (3H) | 2088 | 9.5 | D | D | C |
| Example 8 | | | | | | |
| Comparative | (3I) | 2253 | 9.5 | D | D | C |
| Example 9 | | | | | | |
| Comparative | (3J) | 976 | 9.4 | D | D | D |
| Example 10 | | | | | | |
| Comparative | (3K) | 1244 | 9.5 | C | D | D |
| Example 11 | | | | | | |

As shown in Table 10 to Table 11, the magnetic recording media of Examples 1 to 44 were evaluated as A to B in all evaluation items. Accordingly, it was confirmed that the results of the pickup characteristic test, the spin-off characteristic test, and the corrosion resistance test of the lubricating layers of the magnetic recording media of Examples 1 to 44 were all favorable.

Particularly, the results of the pickup characteristic test and the spin-off characteristic test of the lubricating layers of the magnetic recording media of Examples 1 to 5, 7 to 10, 12 to 27, 29 to 32, and 34 to 44 were all A, which was favorable. In all of the compounds (1A) to (1E), (1G) to (1J), (1L) to (1V), (2A) to (2E), (2G) to (2J), and (2L) to (2V) used in the lubricating layer of the examples, even if the number of carbon atoms contained in R¹—[B]-[A]-and-[C]-[D]-R⁴ was relatively small or even if the number of carbon atoms was large, the distance from the carbon atom as a branch point in the branched terminal group to the primary hydroxy group was not too short and is appropriate. Therefore, it was speculated that an excellent adsorption force for the protective layer exhibited by the secondary hydroxy groups present in R¹—[B]-[A]-and-[C]-[D]-R⁴ and an excellent intermolecular force exhibited by the primary hydroxy groups of the branched terminal group were acted effectively in a well-balanced manner, and the adhesion of the hydroxy groups to the protective layer was more effectively obtained. As a result, it was speculated that adhesion of the lubricant to the magnetic head was minimized and excellent pickup characteristics were exhibited. In addition, it was speculated that the adhesion of the lubricating layer to the protective layer was maintained, and favorable spin-off characteristics were obtained.

On the other hand, as shown in Table 12, the results of the pickup characteristic test and the spin-off characteristic test of the magnetic recording media of Comparative Examples 1 to 11 were all C to D.

In Comparative Examples 1 and 7, the structures corresponding to R¹ and R⁴ in the compound used in the lubricating layer each had only one primary hydroxy group. Therefore, the lubricating layers of Comparative Examples 1 and 7 had less adhesion to the protective layer than the lubricating layers of Examples 1 to 44 containing a compound in which R¹ and R⁴ were a branched terminal group each having two or three primary hydroxy groups. As a result, it was speculated that, in Comparative Examples 1 and 7, the lubricant adhered to the magnetic head, and the result of the pickup characteristic test was C. In addition, it was speculated that, in Comparative Examples 1 and 7, since it was difficult to maintain the adhesion to the protective layer, the result of the spin-off characteristic test was C.

In Comparative Examples 2 to 6 and 9, the structures corresponding to R¹ and R⁴ in the compound used in the lubricating layer did not have a branched terminal group having two primary hydroxy groups, and the two hydroxy groups arranged at both terminals were bonded to adjacent carbon atoms. In the lubricating layer containing the compound used in Comparative Examples 2 to 6 and 9, among the two hydroxy groups arranged at both terminals of the compound, any one hydroxy group was less likely to adhere to the protective layer. As a result, it was speculated that, in Comparative Examples 2 to 6 and 9, the lubricant adhered to the magnetic head, and the result of the pickup characteristic test was D. In addition, it was speculated that the adhesion to the protective layer could not be maintained, and the result of the spin-off characteristic test was D. In addition, it was speculated that, in Comparative Examples 2 to 6, 9, since a compound in which two hydroxy groups arranged at both terminals were bonded to adjacent carbon atoms was used, the adhesion to the protective layer was insufficient, and the result of the corrosion resistance test was C.

In Comparative Example 8, the structures corresponding to R¹ and R⁴ in the compound used in the lubricating layer each had only one primary hydroxy group, and only a divalent linking group corresponding to one of the —[B]-[A]- structure and the —[C]-[D]- structure was present. Therefore, it was speculated that the lubricating layer of Comparative Example 8 had insufficient adhesion to the protective layer. As a result, it was speculated that, in Comparative Example 8, the lubricant adhered to the magnetic head, and the result of the pickup characteristic test was D. In addition, it was speculated that, in Comparative Example 8, the adhesion to the protective layer could not be maintained and the result of the spin-off characteristic test was D. In addition, it was speculated that, in Comparative Example 8, since a compound with one terminal composed of only OH was used, the adhesion to the protective layer was insufficient, and the result of the corrosion resistance test was C.

In Comparative Examples 10 and 11, a compound with a terminal group having two primary hydroxy groups arranged at both terminals of the molecule was used in the lubricating layer. However, the compound used in the lubricating layer in Comparative Examples 10 and 11 did not contain a divalent linking group corresponding to the —[B]-[A]- structure and —[C]-[D]- structure in the fluorine-containing ether compound represented by Formula (1). Therefore, it was speculated that, in the lubricating layers of Comparative Examples 10 and 11, compared to the lubricating layers of Examples 1 to 44, the adsorption force for the protective layer was poor, the result of the pickup characteristic test was C or D, and the result of the spin-off characteristic test was D. In addition, it was speculated that, in Comparative Examples 10 and 11, since the compound used in the lubricating layer had only one PFPE chain, the hydrophobicity of the lubricating layer was insufficient, and the result of the corrosion resistance test was D.

### INDUSTRIAL APPLICABILITY

There is provided a fluorine-containing ether compound which can form a lubricating layer which has favorable adhesion to the protective layer, has a strong effect of inhibiting corrosion of the magnetic recording medium, and can minimize the occurrence of pickup and spin-off even if the thickness is thin, and suitably used as a material for a lubricant for magnetic recording medium.

### REFERENCE SIGNS LIST

10 Magnetic recording medium
11 Substrate
12 Adhesive layer
13 Soft magnetic layer
14 First base layer
15 Second base layer
16 Magnetic layer
17 Protective layer
18 Lubricating layer

1. A fluorine-containing ether compound represented by the following Formula (1): 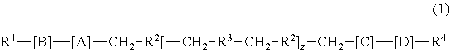
(in Formula (1), [A] is represented by the following Formula (2-1), in Formula (2-1), a is an integer of 0 to 3, [B] is represented by the following Formula (2-2), in Formula (2-2), b is an integer of 0 to 3 and c is an integer of 2 to 5, wherein a sum of the values of a and b is 1 to 3, in Formula (1), [A] and [B] may be interchanged, [C] is represented by the following Formula (3-1), in Formula (3-1), d is an integer of 0 to 2, [D] is represented by the following Formula (3-2), in Formula (3-2), e is an integer of 0 to 2, and f is an integer of 2 to 5, wherein a sum of the values of d and e is 1 or 2, in Formula (1), [C] and [D] may be interchanged, R⁴ is a branched terminal group having 3 to 30 carbon atoms and represented by the following Formula (4), in Formula (4), L represents an integer of 0 to 6, in Formula (4), Y¹ and Y² are each independently a hydrocarbon group which has only one primary hydroxy group and may contain an ether oxygen atom, in Formula (4), Y³ is a hydrocarbon group which has only one primary hydroxy group and may contain an ether oxygen atom, or a hydrogen atom, R¹ is a terminal group that may be the same as or different from R⁴, and a branched terminal group having 3 to 30 carbon atoms represented by Formula (4), an organic group having 1 to 30 carbon atoms, which has an ether oxygen atom at the terminal bonded to [A] or [B], or a hydroxy group, z represents 1 or 2, R² is a perfluoropolyether chain, some or all of two or three R²'s may be the same as or different from each other, R³ is a divalent linking group represented by the following Formula (5), in Formula (5), y1 is an integer of 1 to 3, and y2 is an integer of 1 to 3, in Formula (5), a dotted line bonded to the oxygen atom on the left side indicates a bond with a methylene group on the side of R¹, and a dotted line bonded to the oxygen atom on the right side indicates a bond with a methylene group on the side of R⁴, and when z is 2, two R³'s may be the same as or different from each other).

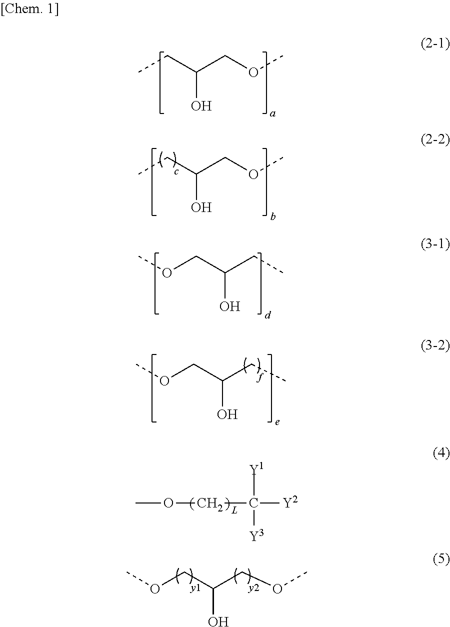

2. The fluorine-containing ether compound according to claim 1,
wherein Formula (4) representing R⁴ in Formula (1) is any of the following Formulae (6-1) to (6-3): 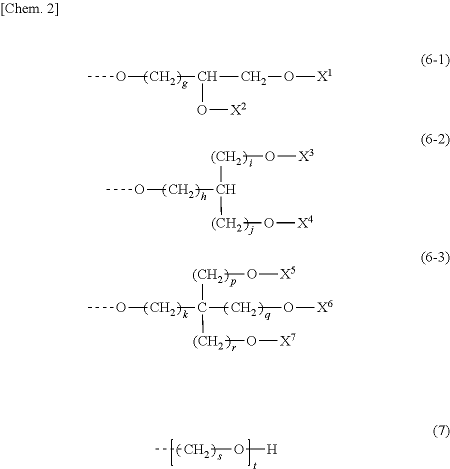
(in Formula (6-1), g represents an integer of 1 to 6, X¹ and X² are represented by Formula (7), and X¹ and X² may be the same as or different from each other)
(in Formula (6-2), h represents an integer of 0 to 6, i and j each independently represent an integer of 1 to 6, X³ and X⁴ represent a hydrogen atom or are represented by Formula (7), and X³ and X⁴ may be the same as or different from each other)
(in Formula (6-3), k represents an integer of 0 to 6, p, q and r each independently represent an integer of 1 to 6, X⁵, X⁶ and X⁷ represent a hydrogen atom or are represented by Formula (7), and X⁵, X⁶ and X⁷ may be different from each other, or some or all of them may be the same)
(in Formula (7), s represents an integer of 2 to 6, and t represents 1 or 2).

3. The fluorine-containing ether compound according to claim 1,
wherein, in Formula (1), R¹ is a branched terminal group having 3 to 30 carbon atoms represented by Formula (4).

4. The fluorine-containing ether compound according to claim 2,
wherein, in Formula (1), both R¹ and R⁴ are a branched terminal group of any of Formulae (6-1) to (6-3).

5. The fluorine-containing ether compound according to claim 1,
wherein, in Formula (1), R¹—[B]-[A]-and-[C]-[D]-R⁴ are the same.

6. The fluorine-containing ether compound according to claim 1,
wherein, in Formula (1), z is 2, and atoms contained in two R³'s are arranged symmetrically with respect to R² arranged in the center of a chain structure of a molecule.

7. The fluorine-containing ether compound according to claim 1,
wherein, in Formula (1), R¹ is represented by the following Formula (8): 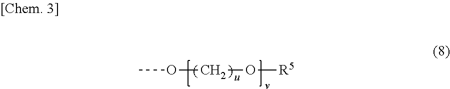
(in Formula (8), u represents an integer of 2 to 6, v represents 0 or 1, and R⁵ is any of a hydrogen atom, an optionally substituted alkyl group containing no hydroxy group, and an organic group having at least one double bond or triple bond, provided that the alkyl group and the organic group may be linear or branched).

8. The fluorine-containing ether compound according to claim 7,
wherein, in Formula (8), R⁵ is an alkyl group having 1 to 6 carbon atoms.

9. The fluorine-containing ether compound according to claim 7,
wherein, in Formula (8), R⁵ is an alkyl group having a substituent and having 1 to 6 carbon atoms, and the substituent is a fluoro group or a cyano group.

10. The fluorine-containing ether compound according to claim 7,
wherein, in Formula (8), R⁵ is any of an aromatic hydrocarbon-containing organic group having 6 to 12 carbon atoms, an aromatic heterocycle-containing organic group having 3 to 10 carbon atoms, an alkenyl group having 2 to 8 carbon atoms, and an alkynyl group having 3 to 8 carbon atoms.

11. The fluorine-containing ether compound according to claim 7,
wherein, in Formula (8), R⁵ is one group selected from the group consisting of a methyl group, ethyl group, n-propyl group, isopropyl group, 2,2,2-trifluoroethyl group, 2,2,3,3,3-pentafluoropropyl group, 2,2,2,2,2,2-hexafluoroisopropyl group, 2-cyanoethyl group, 3-cyanopropyl group, 4-cyanobutyl group, phenyl group, methoxyphenyl group, cyanophenyl group, phenethyl group, thienylethyl group, N-methylpyrazolylmethyl group, allyl group, 3-butenyl group, 4-pentenyl group, propargyl group, 3-butynyl group, and 4-pentynyl group.

12. The fluorine-containing ether compound according to claim 7,
wherein, in Formula (8), R⁵ is a hydrogen atom.

13. The fluorine-containing ether compound according to claim 1,
wherein, in Formula (1), two or three R²'s are each independently a perfluoropolyether chain represented by the following Formula (9): 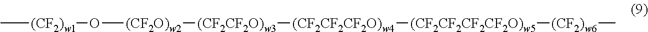
(in Formula (9), w2, w3, w4, and w5 indicate an average degree of polymerization and each independently represent 0 to 20; provided that all of w2, w3, w4, and w5 are not 0 at the same time, w1 and w6 are an average value representing the number of CF₂'s and each independently represent 1 to 3, and the arrangement order of repeating units (CF₂O), (CF₂CF₂O), (CF₂CF₂CF₂O), and (CF₂CF₂CF₂CF₂O) in Formula (9) is not particularly limited).

14. The fluorine-containing ether compound according to claim 1,
wherein, in Formula (1), two or three R²'s are each independently any one selected from among perfluoropolyether chains represented by the following Formulae (10-1) to (10-4): 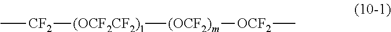
(in Formula (10-1), 1 and m indicate an average degree of polymerization, 1 represents 0.1 to 20, and m represents 0 to 20) 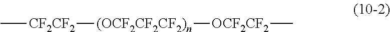
(in Formula (10-2), n indicates an average degree of polymerization and represents 0.1 to 15) 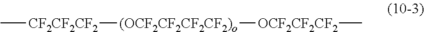
(in Formula (10-3), o indicates an average degree of polymerization and represents 0.1 to 10) 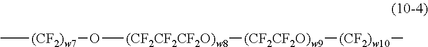
(in Formula (10-4), w8 and w9 indicate an average degree of polymerization and each independently represent 0.1 to 20. w7 and w10 are an average value representing the number of CF₂'s and each independently represent 1 to 2).

15. The fluorine-containing ether compound according to claim 1,
wherein the number-average molecular weight is in a range of 500 to 10,000.

16. A lubricant for magnetic recording medium comprising the fluorine-containing ether compound according to claim 1.

17. A magnetic recording medium in which at least a magnetic layer, a protective layer, and a lubricating layer are sequentially provided on a substrate,
wherein the lubricating layer contains the fluorine-containing ether compound according to claim 1.

18. The magnetic recording medium according to claim 17,
wherein the average film thickness of the lubricating layer is 0.5 nm to 2.0 nm.
